SEQUENCE LISTING (67 sequences)

SEQ ID NO: 1 - DNA, 124 bp, Artificial Sequence
  PRKCB fully methylated MSP amplicon
taagcgtagttggacgagcggtagtagttgggcgagtgatagtttcggtttcgcgcgtcgcggtcgttagagtcggcgtaggggaagcgttcgcggtttcgggtgtagtagcggtcgtcgtttt

SEQ ID NO: 2 - DNA, 241 bp, Artificial Sequence
  ADAMTS12 fully methylated MSP amplicon
acaacgactacaaaactacccgcgatctccctatacttttttaaacaaaaaaaaactaaacacctttttcccctccctcctcctaaaaaaaaaataattcaactaacaatatccgctttcgacgaaatataaaataaaccaaaacgaaaaaacgcaacccaccccgatccccacccctccgcctcccgcataccccgcgacctcgcaacccgcccgctcgatacatcttcctcccgaactc

SEQ ID NO: 3 - DNA, 244 bp, Artificial Sequence
  NAALAD2 fully methylated MSP amplicon
tatttattatgttcgggttattgcgggatttatagaatggaagttgttcgttaataggaagaatgttttttttttttgtagggtttttttttttttatcgagggtttttggggattataggtttttagcgggtagggcggaggcgtggttttgcgaaggttagcggaggttatttagagtttatagttttttgttagcgcgttttttgtttttttgtagtttcgaagttcgcgaatgtagtagg

SEQ ID NO: 4 - DNA, 160 bp, Artificial Sequence
  FILIP1L fully methylated MSP amplicon
cgacctataaacgttacgtcactattctaccttataaaacgctccgcgtatacgacgctatcgacgaaaacgccgataaccgcgaaaccctcgaccgcgacgacgacgcaaccacacaccccaactcccgcgaatattccgaccgtataaacgaaccgta

SEQ ID NO: 5 - DNA, 246 bp, Artificial Sequence
  ZMIZ1 fully methylated MSP amplicon
tcgtttcgaaaattttttaaatcgagatttaatttggatgtttagtttcgtttttttttttttttttttttttttttttcggtcgaggttttttttagtttagtttttttttttatttttttttcgtcgtggatttttattagtatgtttatttgggaggattcgttggggggcgggagatattcgaagttatttatcgttagcgttttttcggcggttttttcgggcgatagcgtttcgggagtt

SEQ ID NO: 6 - DNA, 169 bp, Artificial Sequence
  KCTD8 fully methylated MSP amplicon
ctccgcgtactcctaacgctctacgccctcgaactaaacgacgcgttcctccgaccgaaacgaccccgctcaaaattcgaaacaacgacgacgtcgacgacgcccgaactccatcgaaaaaaaaacgcgcgaaaaaaaaactccgccgatacgacgacgacaataaaaa

SEQ ID NO: 7 - DNA, 164 bp, Artificial Sequence
  CCDC181 fully methylated MSP amplicon
cgaaaacgacaaaaatctacgcaaacgcatacaatatcctcaaaccaccgacccctcccgacacccatcccgatacttacgaaaaaccaataaaactaaaatttctaaaaaaacgactaaaaaatccacgcatcactaacgttataaaaactcgcgaaataccg

SEQ ID NO: 8 - DNA, 145 bp, Artificial Sequence
  PRKCB fully methylated QMSP amplicon
cgtagttggggttagcggtgttaagcgtagttggacgagcggtagtagttgggcgagtgatagtttcggtttcgcgcgtcgcggtcgttagagtcggcgtaggggaagcgttcgcggtttcgggtgtagtagcggtcgtcgtttt

SEQ ID NO: 9 - DNA, 138 bp, Artificial Sequence
  ADAMTS12 fully methylated QMSP amplicon
tcaactaacaatatccgctttcgacgaaatataaaataaaccaaaacgaaaaaacgcaacccaccccgatccccacccctccgcctcccgcataccccgcgacctcgcaacccgcccgctcgatacatcttcctcccg

SEQ ID NO: 10 - DNA, 139 bp, Artificial Sequence
  NAALAD2 fully methylated QMSP amplicon
tgcgaaggttagcggaggttatttagagtttatagttttttgttagcgcgttttttgtttttttgtagtttcgaagttcgcgaatgtagtaggcgttttaagttcggtttttaagaagttatggcggaatttaggggtc

SEQ ID NO: 11 - DNA, 131 bp, Artificial Sequence
  CCDC181 fully methylated QMSP amplicon
tatcctcaaaccaccgacccctcccgacacccatcccgatacttacgaaaaaccaataaaactaaaatttctaaaaaaacgactaaaaaatccacgcatcactaacgttataaaaactcgcgaaataccgc

SEQ ID NO: 12 - DNA, 100 bp, Artificial Sequence
  MT1E fully methylated QMSP amplicon
ggaggagggtggaaggtaatttcggggaaattgggaaaggcggtttggatttcgggaatatcgcgtatttgcgggggtatagttttattcgagcgaacgg

SEQ ID NO: 13 - DNA, 74 bp, Artificial Sequence
  APC fully methylated QMSP amplicon
gaaccaaaacgctccccattcccgtcgaaaacccgccgattaactaaatataaacgcacgtaaccgacatataa

SEQ ID NO: 14 - DNA, 75 bp, Artificial Sequence
  RASSF1 fully methylated QMSP amplicon
cccgtacttcgctaactttaaacgctaacaaacgcgaaccgaacgaaaccacaaaacgaaccccgacttcaacgc

SEQ ID NO: 15 - DNA, 133 bp, Artificial Sequence
  ACTB reference QMSP amplicon
aaccaataaaacctactcctcccttaaaaattacaaaaaccacaacctaataaaaaaaataaccaccacccaacacacaataacaaacacaaattcacaatccaaaaaacttactaaacctcctccatcacca

SEQ ID NO: 16 - DNA, 19 bp, Artificial Sequence
  PRKCB methylated sense primer
taagcgtagttggacgagc

SEQ ID NO: 17 - DNA, 20 bp, Artificial Sequence
  PRKCB methylated antisense primer
aaaacgacgaccgctactac

SEQ ID NO: 18 - DNA, 22 bp, Artificial Sequence
  PRKCB unmethylated sense primer
tgttaagtgtagttggatgagt

SEQ ID NO: 19 - DNA, 23 bp, Artificial Sequence
  PRKCB unmethylated antisense primer
aaaacaacaaccactactacacc

SEQ ID NO: 20 - DNA, 22 bp, Artificial Sequence
  ADAMTS12 methylated sense primer
gagttcgggaggaagatgtatc

SEQ ID NO: 21 - DNA, 22 bp, Artificial Sequence
  ADAMTS12 methylated antisense primer
acaacgactacaaaactacccg

SEQ ID NO: 22 - DNA, 22 bp, Artificial Sequence
  ADAMTS12 unmethylated sense primer
gagtttgggaggaagatgtatt

SEQ ID NO: 23 - DNA, 23 bp, Artificial Sequence
  ADAMTS12 unmethylated antisense primer
aaacaacaactacaaaactacca

SEQ ID NO: 24 - DNA, 24 bp, Artificial Sequence
  NAALAD2 methylated sense primer
tatttattatgttcgggttattgc

SEQ ID NO: 25 - DNA, 21 bp, Artificial Sequence
  NAALAD2 methylated antisense primer
cctactacattcgcgaacttc

SEQ ID NO: 26 - DNA, 26 bp, Artificial Sequence
  NAALAD2 unmethylated sense primer
gttatttattatgtttgggttattgt

SEQ ID NO: 27 - DNA, 23 bp, Artificial Sequence
  NAALAD2 unmethylated antisense primer
cctactacattcacaaacttcaa

SEQ ID NO: 28 - DNA, 20 bp, Artificial Sequence
  FILIP1L methylated sense primer
tacggttcgtttatacggtc

SEQ ID NO: 29 - DNA, 21 bp, Artificial Sequence
  FILIP1L methylated antisense primer
cgacctataaacgttacgtca

SEQ ID NO: 30 - DNA, 25 bp, Artificial Sequence
  FILIP1L unmethylated sense primer
ggaattatggtttgtttatatggtt

SEQ ID NO: 31 - DNA, 24 bp, Artificial Sequence
  FILIP1L unmethylated antisense primer
cccaacctataaacattacatcac

SEQ ID NO: 32 - DNA, 23 bp, Artificial Sequence
  ZMIZ1 methylated sense primer
tcgtttcgaaaattttttaaatc

SEQ ID NO: 33 - DNA, 18 bp, Artificial Sequence
  ZMIZ1 methylated antisense primer
aactcccgaaacgctatc

SEQ ID NO: 34 - DNA, 27 bp, Artificial Sequence
  ZMIZ1 unmethylated sense primer
tgtagtttgttttgaaaattttttaaa

SEQ ID NO: 35 - DNA, 21 bp, Artificial Sequence
  ZMIZ1 unmethylated antisense primer
aactcccaaaacactatcacc

SEQ ID NO: 36 - DNA, 22 bp, Artificial Sequence
  KCTD8 methylated sense primer
tttttattgtcgtcgtcgtatc

SEQ ID NO: 37 - DNA, 18 bp, Artificial Sequence
  KCTD8 methylated antisense primer
ctccgcgtactcctaacg

SEQ ID NO: 38 - DNA, 25 bp, Artificial Sequence
  KCTD8 unmethylated sense primer
gtttttttattgttgttgttgtatt

SEQ ID NO: 39 - DNA, 21 bp, Artificial Sequence
  KCTD8 unmethylated antisense primer
accctccacatactcctaaca

SEQ ID NO: 40 - DNA, 24 bp, Artificial Sequence
  CCDC181 methylated sense primer
cggtatttcgcgagtttttataac

SEQ ID NO: 41 - DNA, 21 bp, Artificial Sequence
  CCDC181 methylated antisense primer
cgaaaacgacaaaaatctacg

SEQ ID NO: 42 - DNA, 27 bp, Artificial Sequence
  CCDC181 unmethylated sense primer
tagtggtattttgtgagtttttataat

SEQ ID NO: 43 - DNA, 24 bp, Artificial Sequence
  CCDC181 unmethylated antisense primer
acaaaaacaacaaaaatctacaca

SEQ ID NO: 44 - DNA, 20 bp, Artificial Sequence
  PRKCB methylated sense primer
cgtagttggggttagcggtg

SEQ ID NO: 45 - DNA, 21 bp, Artificial Sequence
  PRKCB methylated antisense primer
aaaacgacgaccgctactaca

SEQ ID NO: 46 - DNA, 23 bp, Artificial Sequence
  PRKCB hydrolysis probe
  misc_feature (1)..(1): JOE
  misc_feature (23)..(23): BHQ1 (black hole quencher 1)
ttagagtcggcgtaggggaagcg

SEQ ID NO: 47 - DNA, 21 bp, Artificial Sequence
  ADAMTS12 methylated sense primer
cgggaggaagatgtatcgagc

SEQ ID NO: 48 - DNA, 23 bp, Artificial Sequence
  ADAMTS12 methylated antisense primer
tcaactaacaatatccgctttcg

SEQ ID NO: 49 - DNA, 27 bp, Artificial Sequence
  ADAMTS12 hydrolysis probe
  misc_feature (1)..(1): CY5
  misc_feature (27)..(27): BHQ3 (black hole quencher 3)
tttcgttttggtttattttatatttcg

SEQ ID NO: 50 - DNA, 19 bp, Artificial Sequence
  NAALAD2 methylated sense primer
tgcgaaggttagcggaggt

SEQ ID NO: 51 - DNA, 21 bp, Artificial Sequence
  NAALAD2 methylated antisense primer
gacccctaaattccgccataa

SEQ ID NO: 52 - DNA, 23 bp, Artificial Sequence
  NAALAD2 hydrolysis probe
  misc_feature (1)..(1): FAM
  misc_feature (23)..(23): BHQ1 (black hole quencher 1)
gaagttcgcgaatgtagtaggcg

SEQ ID NO: 53 - DNA, 22 bp, Artificial Sequence
  CCDC181 methylated sense primer
gcggtatttcgcgagtttttat

SEQ ID NO: 54 - DNA, 19 bp, Artificial Sequence
  CCDC181 methylated antisense primer
tatcctcaaaccaccgacc

SEQ ID NO: 55 - DNA, 22 bp, Artificial Sequence
  CCDC181 hydrolysis probe
  misc_feature (1)..(1): FAM
  misc_feature (22)..(22): BHQ1 (black hole quencher 1)
agtatcgggatgggtgtcggga

SEQ ID NO: 56 - DNA, 20 bp, Artificial Sequence
  MT1E methylated sense primer
ggaggagggtggaaggtaat

SEQ ID NO: 57 - DNA, 21 bp, Artificial Sequence
  MT1E methylated antisense primer
ccgttcgctcgaataaaacta

SEQ ID NO: 58 - DNA, 24 bp, Artificial Sequence
  MT1E hydrolysis probe
  misc_feature (1)..(1): JOE
  misc_feature (24)..(24): BHQ1 (black hole quencher 1)
atttcgggaatatcgcgtatttgc

SEQ ID NO: 59 - DNA, 19 bp, Artificial Sequence
  APC methylated sense primer
gaaccaaaacgctccccat

SEQ ID NO: 60 - DNA, 27 bp, Artificial Sequence
  APC methylated antisense primer
ttatatgtcggttacgtgcgtttatat

SEQ ID NO: 61 - DNA, 22 bp, Artificial Sequence
  APC hydrolysis probe
  misc_feature (1)..(1): CY5
  misc_feature (22)..(22): BHQ3 (black hole quencher 3)
cccgtcgaaaacccgccgatta

SEQ ID NO: 62 - DNA, 18 bp, Artificial Sequence
  RASSF1 methylated sense primer
gcgttgaagtcggggttc

SEQ ID NO: 63 - DNA, 24 bp, Artificial Sequence
  RASSF1 methylated antisense primer
cccgtacttcgctaactttaaacg

SEQ ID NO: 64 - DNA, 24 bp, Artificial Sequence
  RASSF1 hydrolysis probe
  misc_feature (1)..(1): FAM
  misc_feature (24)..(24): BHQ1 (black hole quencher 1) or TAMRA
acaaacgcgaaccgaacgaaacca

SEQ ID NO: 65 - DNA, 25 bp, Artificial Sequence
  ACTB reference sense primer
tggtgatggaggaggtttagtaagt

SEQ ID NO: 66 - DNA, 27 bp, Artificial Sequence
  ACTB reference antisense primer
aaccaataaaacctactcctcccttaa

SEQ ID NO: 67 - DNA, 30 bp, Artificial Sequence
  ACTB hydrolysis probe
  misc_feature (1)..(1): FAM
  misc_feature (30)..(30): BHQ1 (black hole quencher 1) or TAMRA
accaccacccaacacacaataacaaacaca

(19) United States
(12) Patent Application Publication
(10) Pub. No.: US 2022/0267858 A1
(43) Pub. Date: Aug. 25, 2022

(54) CHARACTERIZATION OF PROSTATE CANCER USING DNA METHYLATION ASSAY SYSTEM

(71) Applicant: Vinius University, Vilnius (LT)

(72) Inventors: Kristina DANIUNAITE, Vilnius (LT); Sonata JARMALAITE, Vilnius (LT)

(21) Appl. No.: 17/628,242

(22) PCT Filed: Jul. 19, 2019
(86) PCT No.: PCT/IB2019/056204

§ 371 Date: Jan. 19, 2022

(51) Int. Cl. C12Q 1/6886

(52) U.S. Cl. CPC C12Q 1/6886; C12Q 2600/16; C12Q 2600/154; C12Q 2600/112

(57) ABSTRACT

The present invention—PROMESYS—relates to methods and tools for diagnosis and prognosis of prostate cancer, patient monitoring/follow-up and prediction of response to treatment of patients with confirmed diagnosis of prostate cancer. The methods conducted in vitro comprise the steps of providing a tissue and/or a body fluid sample, obtained from an individual, and determining DNA methylation status and/or level of one or more genes selected from the group consisting of PRKCB, ADAMTS12, NAALAD2, FILIP1L, ZMIZ1 and KCTD8 in a sample. Additionally, methylation status and/or level of CCDC181, MT1E, APC and/or RASSF1 can be included in the biomarker panel for improved performance. Furthermore, the present invention refers to kits and oligonucleotides for use in such a method.

### FIELD OF THE INVENTION

The present invention relates to the area of prostate cancer (PCa) assessment in an individual suspected of having PCa, having predisposition to PCa or diagnosed with any stage PCa. In particular, it relates to biomarkers and methods for identifying PCa, diagnosing PCa, predicting PCa progression, predicting response to treatment, monitoring the efficacy of the treatment in an individual having developed PCa, whereby DNA methylation status and/or level of particular epigenetic biomarkers are detected and measured in vitro in prostate tissue and/or body fluids, like urine, or a combination thereof. The invention also relates to kits for performing the assays and the use of certain oligonucleotides in the assays.

### BACKGROUND OF THE INVENTION

PCa is the most prevalent malignancy and the 5^{th} most common cause of male cancer deaths worldwide, with the highest incidence and mortality rates in developed countries [according to 2018 data from Cancer Today, Global Cancer Observatory, International Agency for Research on Cancer; https://gco.iarc.fr/today/home]. In general, PCa is a slowly developing malignancy, which may remain indolent for decades. However, while many PCa lesions remain localized, a subset shows an aggressive course with rapid development of metastases, which is fatal within a short time following diagnosis [1,2]. PCa is considered to be a multifocal malignancy due to the common presence of multiple primary tumours (found in 60-90% of cases) that are histologically independent and often genetically distinct [3,4,5]. This reflects the underlying heterogeneity of PCa and raises the need not only to accurately and timely diagnose the disease, but also to determine its further course and to select the most proper treatment strategy as early as possible.

Men are diagnosed with PCa at various stages of the disease and with very different prognoses and, thus, a wide selection of treatment modalities are available. Treatment options are mainly based on tumour stage and cell differentiation, with regard to symptoms and patient's quality of life, but usually without considering the molecular profile of the tumour. The current inability to distinguish aggressive from indolent/latent PCa at diagnosis remains one of the major clinical challenges of PCa treatment [6,7].

Biochemical disease recurrence (BCR), described by a rising prostate-specific antigen (PSA) level in blood after treatment, is usually the first sign indicating treatment failure and preceding metastatic progression [8]. The progression of advanced PCa to castration-resistant prostate cancer (CRPC) in 1-2 years is inevitable and ultimately fatal [9]. The treatment of CRPC is based on expensive systemic therapy, like next-generation targeted therapy (e.g. abiraterone acetate (AA), enzalutamide), chemotherapy, and etc., but only a part of CRPC patients respond to this treatment positively, while others have primary resistance [9]. Furthermore, the benefits of androgen receptor (AR) pathway-directed therapies are usually short-lived and secondary resistance occurs invariably, leading to an incurable disease. Elevated PSA indicates disease relapse only after its actual occurrence, which means that PCa has already developed into the stage eventually leading to death. Therefore, biomarkers that could not only predict PCa progression, but also be able to lead the way for personalized treatment decisions, as well as enable patient's monitoring during the treatment, are needed.

Epigenetic modifications are defined as reversible biochemical changes affecting gene expression without altering the primary DNA sequence. DNA methylation at the 5′ carbon of cytosine (5-mC) in cytosine-guanine dinucleotides (CpGs) is the most intensively studied epigenetic mechanism for control of gene expression, which is nearly ubiquitous in multicellular organisms and is essential for the normal development in mammals. CpGs in the genome are distributed unevenly: more than half of the genes contain short CpG-rich regions known as CpG islands, while the rest of the genome is depleted for CpGs [10]. CpG islands span transcription start sites (TSS) of roughly half of the human genes, often overlap with promoters and other regulatory sequences and, therefore, mostly represent genes which are actively expressed or poised for transcription [11].

Aberrant DNA methylation at CpG islands is frequent in cancer, including PCa, and is often associated with the silencing of tumour suppressor genes and downstream signalling pathways, leading to cancer development and progression [12]. During prostate carcinogenesis epigenetic changes in tumour suppressor genes occur earlier than genetic aberrations and are more consistent among tumours than mutations. Various studies have reported over 100 genes which show altered DNA methylation patterns in tumours as compared to benign prostatic samples. However, because of high inter-individual variations, only some of them are currently recognized as putative diagnostic biomarkers of PCa, most notably glutathione S-transferase pi 1 (GSTP1), RAS association domain family member 1 (RASSF1), and a few other genes, while evident prognostic and predictive DNA methylation biomarkers are scarce.

Regarding the implementation possibilities in clinical practice, DNA methylation has several advantages over other commonly used biomarkers. In contrast to RNA transcripts and most proteins, DNA is much more stable both in vivo and ex vivo, and can withstand harsh conditions for prolonged periods. Moreover, methylated DNA can be amplified for increased sensitivity, thus, allowing measurements on limited amounts of test samples. PCa-derived methylated DNA is easily detectable in body fluids, such as urine [13], blood [14], plasma [15] or other sample types. This allows for the development of non-invasive or minimally invasive molecular tests, which are expected to replace or at least to augment the use of invasive biopsy. Liquid biopsy could be scheduled more frequently, which is especially important during PCa treatment through providing timely evidence of disease recurrence or resistance [16]. DNA methylation in body fluids from early stage PCa patients can better reflect all tumour foci unlike tissue biopsy, which poorly accounts for PCa heterogeneity. Regarding prostate anatomy and the common tumour localization in its peripheral zone, urine is the most suitable body fluid for liquid biopsy in case of PCa testing as it is easily obtainable and biomarkers are less diluted than in serum or plasma [15].

The present invention identifies a set of DNA methylation biomarkers showing the potential clinical benefit and develops a strategy for stratification of PCa patients according to the potential of the disease progression and treatment selection. It also provides a putative non-invasive tool for the patients' monitoring during a particular treatment.

### SUMMARY OF THE INVENTION

The present disclosure (PROMESYS) provides a solution to solve the problems of the related art by analysing one or more of the group of DNA methylation-based biomarkers. More specifically, we have identified that alterations of DNA methylation status and/or DNA methylation level of a set of genomic loci including the genes PRKCB, ADAMTS12, NAALAD2, FILIP1L, ZMIZ1 and KCTD8 are associated with prostate cancer (PCa) and can be used as the biomarkers for PCa detection, diagnostics and prognosis, patients' follow-up/monitoring and/or assessment of the response to treatment when analysed individually or in various combinations. Additionally, other epigenetic biomarkers, namely CCDC181, MT1E, APC and RASSF1, can be included in the biomarker panel providing increased diagnostic and/or prognostic value of the test. Exemplary drugs for which the patient's responsiveness can be assessed by the provided methods include abiraterone acetate (AA), docetaxel (DTX) and derivatives or analogues thereof.

According to one embodiment of the invention a group of DNA methylation biomarkers consisting of PRKCB (SEQ ID NO: 1), ADAMTS12 (SEQ ID NO: 2), NAALAD2 (SEQ ID NO: 3), FILIP1L (SEQ ID NO: 4), ZMIZ1 (SEQ ID NO: 5) and KCTD8 (SEQ ID NO: 6) is provided for identification and/or characterization of PCa, and/or prognosis of PCa progression in a test sample containing prostate tissue, prostate cells or nucleic acids from prostate tissue or cells obtained from an individual. Additionally, CCDC181 (SEQ ID NO: 7) can be included in the panel.

According to another embodiment of the invention a method, based on qualitative methylation-specific PCR (MSP), is provided for identification of at least one of the DNA methylation biomarkers from the group consisting of PRKCB (SEQ ID NO: 1), ADAMTS12 (SEQ ID NO: 2), NAALAD2 (SEQ ID NO: 3), FILIP1L (SEQ ID NO: 4), ZMIZ1 (SEQ ID NO: 5), KCTD8 (SEQ ID NO: 6) and CCDC181 (SEQ ID NO: 7) in a test sample containing prostate tissue, prostate cells, body fluid obtained from an individual or nucleic acids from prostate cells or body fluid.

Another embodiment of the invention provides a second group of biomarkers consisting of PRKCB (SEQ ID NO: 8), ADAMTS12 (SEQ ID NO: 9) and NAALAD2 (SEQ ID NO: 10) for identification or diagnosis of PCa, characterization of PCa, prognosis of PCa progression, prediction of the response to treatment and/or development of the treatment resistance, follow-up of individuals diagnosed with PCa or being at risk of PCa development and/or monitoring of individuals diagnosed with PCa who are undergoing treatment. Additionally, CCDC181 (SEQ ID NO: 11), MT1E (SEQ ID NO: 12), APC (SEQ ID NO: 13) and RASSF1 (SEQ ID NO: 14) can be included in the biomarker panel.

In another embodiment, the present invention provides a second method, based on quantitative methylation-specific PCR (QMSP), for identification of at least one of the DNA methylation biomarkers from the group consisting of PRKCB (SEQ ID NO: 8), ADAMTS12 (SEQ ID NO: 9) and NAALAD2 (SEQ ID NO: 10), as well as CCDC181 (SEQ ID NO: 11), MT1E (SEQ ID NO: 12), APC (SEQ ID NO: 13) and RASSF1 (SEQ ID NO: 14), known to be associated with PCa, in a test sample containing prostate tissue, prostate cells, body fluid (preferably urine or plasma) or nucleic acids from prostate tissue, prostate cells or body fluid obtained from an individual.

In another aspect, the invention provides a method for obtaining a derivative estimate for assessing DNA methylation of a combination of at least two biomarkers analysed using the quantitative methylation-specific PCR-based method in a test sample containing prostate tissue, prostate cells, body fluid (preferably urine or plasma) or nucleic acids from prostate tissue, prostate cells or body fluid obtained from an individual.

In another aspect, the invention provides kits for assessing qualitatively or/and quantitatively at least one of the methylation biomarkers from the group consisting of PRKCB (SEQ ID NO: 1 or/and SEQ ID NO: 8), ADAMTS12 (SEQ ID NO: 2 or/and SEQ ID NO: 9), NAALAD2 (SEQ ID NO: 3 or/and SEQ ID NO: 10), FILIP1L (SEQ ID NO: 4), ZMIZ1 (SEQ ID NO: 5) and KCTD8 (SEQ ID NO:6) in a test sample containing prostate tissue, prostate cells, body fluid (preferably urine or plasma) or nucleic acids from prostate tissue, prostate cells or body fluid obtained from an individual. The kits can also be used to assess methylation of CCDC181 (SEQ ID NO: 7 or/and SEQ ID NO: 11), MT1E (SEQ ID NO: 12), APC (SEQ ID NO: 13) and RASSF1 (SEQ ID NO: 14) individually, in any internecine combinations or combinations with the previous biomarkers (SEQ ID NOs: 1-6 and SEQ ID NOs: 8-10).

An additional aspect of the invention provides primers and probes for the identification of the methylation biomarkers in a test sample of any kind of human-derived tissue, cells, body fluid or nucleic acids obtained from human-derived tissue, cells or body fluid. A particular primer or probe comprises a nucleotide sequence selected from the group consisting of SEQ ID NOs: 16-58.

### BRIEF DESCRIPTION OF THE FIGURES

The invention is illustrated with the following figures.

FIG. 1. Venn diagrams of the genes with significantly different methylation levels at promoter and intragenic regions according to prostate tissue histology and prostate cancer (PCa) progression status. The lists of differentially methylated genes were obtained by DNA methylation microarray-based analysis. NPT—noncancerous prostate tissue, BCR+/−—biochemical disease recurrence status (positive/negative).

FIG. 2. Gene set enrichment analysis (GSEA) of differentially methylated genes identified in the genome-wide methylation profiling. Only genes having significant methylation differences with fold change values ≥1.2 are included. The collection of Hallmark gene sets (pathways) as defined in MSigDB (http://software.broadinstitute.org/gsea/) were selected for the enrichment analysis. The grey shade intensity indicates the false discovery rate (FDR)-adjusted P-value (q-value). PCa—prostate cancer, NPT—noncancerous prostate tissue, BCR+/−—biochemical disease recurrence status (positive/negative), prom—promoter regions, intra—intragenic regions.

FIG. 3. Volcano plots of DNA methylation profiling in tissues of prostate cancer (PCa) patients. A—methylation differences between PCa and noncancerous tissues; B—methylation differences in tumours of biochemical disease recurrence (BCR)-positive and BCR-negative cases. All probes are depicted as grey-shaded squares coloured according to the cut-off fold change values (FC ≥1.2) and P-values (<0.0500). Labels indicate microarray probes of the genes selected for further validation analysis.

FIG. 4. Methylation frequencies of the genes PRKCB, ADAMTS12, NAALAD2, FILIP1L, ZMIZ1, KCTD8 and CCDC181 in prostate tissues. The results were obtained by means of the qualitative methylation-specific PCR (MSP). PCa—prostate tumours, NPT—noncancerous prostate tissues, BPH—benign prostatic hyperplasia. Significant P-values are in bold.

FIG. 5. Methylation levels of the genes PRKCB, ADAMTS12, NAALAD2 and CCDC181 in prostate tissue samples according to tissue histology. The results were obtained by means of the quantitative methylation-specific PCR (QMSP). The box extends from the 25^{th} to 75^{th} percentiles; the line in the box is plotted at median; the plus sign depicts the mean; the whiskers represent the range. PCa—prostate tumours, BPH—benign prostatic hyperplasia. Significant P-values are in bold.

FIG. 6. Methylation levels of the genes PRKCB, ADAMTS12, NAALAD2 and CCDC181 in prostate tissue samples according to the gene promoter methylation status identified by the qualitative method. The methylation level values of the genes were obtained by means of the quantitative methylation-specific PCR (QMSP), whereas the methylation status was determined using the qualitative methylation-specific PCR. The box extends from the 25^{th} to 75^{th} percentiles; the line in the box is plotted at median; the plus sign depicts the mean; the whiskers represent the range. M/U—methylated/unmethylated gene methylation status. Significant P-values are in bold.

FIG. 7. Methylation levels of the genes PRKCB, ADAMTS12, NAALAD2, FILIP1L, ZMIZ1, KCTD8 and CCDC181 in the prostate cancer dataset (PRAD) of The Cancer Genome Atlas (TCGA). Level 3 DNA methylation data, obtained using Illumina HumanMethylation450K platform, was used to generate the plots. The box extends from the 25^{th} to 75^{th} percentiles; the line in the box is plotted at median; the plus sign depicts the mean; the whiskers represent the 10-90% range; data values outside the range are marked as dots. Significant P-values are in bold.

FIG. 8. Receiver Operating Characteristic (ROC) curve analysis of the prostate cancer dataset (PRAD) of The Cancer Genome Atlas (TCGA) according to the genes PRKCB (A), ADAMTS12 (B), NAALAD2 (C), FILIP1L (D), ZMIZ1 (E), KCTD8 (F) and CCDC181 (G). Level 3 DNA methylation data, obtained using Illumina HumanMethylation450K platform, was used to generate the plots. Area under the curve (AUC) is shown in grey. Significant P-values are in bold.

FIG. 9. Methylation frequencies of the genes PRKCB, ADAMTS12, NAALAD2, FILIP1L, ZMIZ1, KCTD8 and CCDC181 in prostate tumour tissues according to the grade groups by the International Society of Urologic Pathology (ISUP; A), pathological tumour stage pT (B) and TMPRSS2-ERG fusion transcript status (C). Significant P-values are in bold.

FIG. 10. Relative expression levels of the genes PRKCB, ADAMTS12, NAALAD2, ZMIZ1 and CCDC181 in the prostate tissues. A-E—expression of the genes in the prostate tumours (PCa), noncancerous prostate tissues (NPT) and benign prostatic hyperplasia (BPH) samples; F-J—expression of the genes in prostate tissues according to the methylated/unmethylated (M/U) promoter status. The box extends from the 25^{th} to 75^{th} percentiles; the line in the box is plotted at median; the plus sign depicts the mean; the whiskers represent the range. Significant P-values are in bold.

FIG. 11. Relative expression levels of the genes PRKCB (A), ADAMTS12 (B), NAALAD2 (C), ZMIZ1 (D) and CCDC181 (E) in the prostate cancer cohort (PRAD) of The Cancer Genome Atlas (TCGA). Level 3 PRAD RNA-seq RSEM data were used to generate the plots. The box extends from the 25^{th} to 75^{th} percentiles; the line in the box is plotted at median; the plus sign depicts the mean; the whiskers represent the 10-90% range; data values outside the range are marked as dots. PCa—prostate cancer. Significant P-values are in bold.

FIG. 12. Correlations between promoter methylation and gene expression levels for PRKCB (A), ADAMTS12 (B), NAALAD2 (C), ZMIZ1 (D) and CCDC181 (E) in the prostate cancer cohort (PRAD) of The Cancer Genome Atlas (TCGA). Level 3 DNA methylation data obtained using Illumina HumanMethylation450K platform, and level 3 PRAD RNA-seq RSEM data were used to generate scatter plots. RNA-seq data is plotted on log 2 scale. For easier visual comparison, Oy axis is at the same range in all parts of the figure. Pearson's R (R_{P}) and Spearman's R (R_{S}) correlation coefficients are provided with respective P-values. Significant P-values are in bold.

FIG. 13. Methylation frequencies of the genes PRKCB, ADAMTS12, NAALAD2, FILIP1L, ZMIZ1, KCTD8 and CCDC181 according to the biochemical disease recurrence (BCR) status. A—all prostate tumours; B—tumours with ISUP grade groups 1 or 2 only. Significant P-values are in bold.

FIG. 14. Methylation frequencies of the genes PRKCB, ADAMTS12, NAALAD2, FILIP1L, ZMIZ1, KCTD8 and CCDC181 according to the biochemical disease recurrence (BCR) status in prostate tumours with (A) and without (B) TMPRSS2-ERG gene fusion transcript. Significant P-values are in bold.

FIG. 15. Kaplan-Meier curve analysis of methylation status of (A) PRKCB, (B) ADAMTS12, (C) NAALAD2, (D) FILIP1L, (E) ZMIZ1, (F) KCTD8 and (G) CCDC181 in prostate tissues for predicting biochemical disease recurrence (BCR) after radical prostatectomy (RP). M/U—methylated/unmethylated promoter status. Significant P-values are in bold.

FIG. 16. Correlations between quantitative DNA methylation estimates obtained by single-assay and multiplex experiments in various samples. A—methylation levels of PRKCB, B—methylation levels of ADAMTS12, C—derivative methylation estimates xMI of the two genes. Randomly selected tissue and urine samples were used for the analysis (four of each type). Two urine samples with values [0; 0] are overlapping at intersection. PCa—prostate cancer (localized or locally advanced cases), CRPC—castration-resistant PCa, R_{P}—Pearson's correlation coefficient, R_{S}—Spearman's correlation coefficient. Significant P-values are in bold.

FIG. 17. Methylation levels in urine of patients diagnosed with localized or locally advanced prostate cancer (PCa) and control cases. BPH—benign prostatic hyperplasia, ASC—asymptomatic (healthy) males. Whiskers represent the standard error of mean. Significant P-values are in bold.

FIG. 18. Methylation levels in urine of patients diagnosed with localized or locally advanced prostate cancer according to the biochemical disease recurrence (BCR) status. Boxes indicate interquartile range with median values depicted as lines, whiskers represent the range. Significant P-values are in bold.

FIG. 19. Kaplan-Meier curve analysis of methylation status of (A) PRKCB, (B) ADAMTS12, (C) NAALAD2, (D) CCDC181, (E) combination of PRKCB, ADAMTS12 and NAALAD2, and (F) combination of PRKCB, ADAMTS12, NAALAD2 and CCDC181 in prostate tissues for predicting biochemical disease recurrence (BCR) after radical prostatectomy (RP). M/U—methylated/unmethylated promoter status. Significant P-values are in bold.

FIG. 20. Receiver Operating Characteristic (ROC) curve analysis of the methylation biomarkers, as predictors of biochemical disease progression, in urine of the patients diagnosed with localized or locally advance prostate cancer (PCa). A—PRKCB, B—ADAMTS12, C—NAALAD2, D—CCDC181, E—xMI3 and F—xMI4. xMI3—the derivative methylation estimate based on biomarkers PRKCB, ADAMTS12 and NAALAD2; xMI4—the derivate methylation estimate based on the biomarkers PRKCB, ADAMTS12, NAALAD2 and CCDC181. AUC—area under the curve. Significant P-values are in bold.

FIG. 21. Differences of the gene methylation levels (A) and methylation frequencies (B) in urine of patients diagnosed with castration-resistant prostate cancer (CRPC) according to the prior radical treatment status. Only urine samples collected before initiating the 1-line therapy are included. Whiskers indicate the standard error of mean (SEM). Significant P-values are in bold.

FIG. 22. Differences of the gene methylation levels (A) and methylation estimates xMI (B) in urine collected before initiating the abiraterone acetate (AA) treatment according to the castration-resistant prostate cancer (CRPC) progression status. xMI3—the derivative methylation estimate based on the biomarkers PRKCB, ADAMTS12 and NAALAD2; xMI4—the derivate methylation estimate based on the biomarkers PRKCB, ADAMTS12, NAALAD2 and CCDC181. Significant P-values are in bold.

FIG. 23. Kaplan-Meier curve analysis of the gene methylation status in urine samples of castration-resistant prostate cancer (CRPC) cases collected during the treatment with abiraterone acetate (AA). Several selected assays are shown. Urine samples were collected at least 2 mos. after the initiation of the treatment. Time to progression was calculated from the sample collection date. M/U—methylated/unmethylated promoter status; RT—radical treatment. Significant P-values are in bold.

FIG. 24. Differences of methylation estimate xMI3 and xMI4 levels in urine, collected before initiating the treatment with abiraterone acetate (AA), according to the PSA progression status. A—analysis of all the samples; B—analysis of the samples from the patients with long response (>3 yrs.) to prior hormonal therapy (HT). The absence of the prostate-specific antigen (PSA) level reduction by ≥50% was considered as the PSA progression. Significant P-values are in bold.

FIG. 25. Differences of the biomarker methylation levels (A) and methylation estimate xMI levels (B) in urine, collected before initiating the treatment with abiraterone acetate (AA), according to primary resistance to abiraterone acetate (AA) status. Significant P-values are in bold.

FIG. 26. Differences of the biomarker methylation levels (A) and methylation estimate xMI levels (B) in urine, collected before initiating the treatment with abiraterone acetate (AA), according to the primary resistance to abiraterone acetate (AA) status. Only castration-resistant prostate cancer (CRPC) cases with short response (≤3 yr.) to hormonal therapy (HT) are included in the analysis. Significant P-values are in bold.

FIG. 27. Differences of the biomarker methylation levels (A) and methylation estimate xMI levels (B) in urine, collected before initiating the treatment with abiraterone acetate (AA), according to the resistance to abiraterone acetate (AA) type/status. Only castration-resistant prostate cancer (CRPC) cases with short response (≤3 yrs.) to hormonal therapy (HT) are included in the analysis. Significant P-values are in bold.

FIG. 28. Methylation frequencies of the biomarkers in urine samples collected during the treatment with abiraterone acetate (AA) according to the death status. Urine samples were collected at least 2 mos. after the initiation of the treatment. Significant P-values are in bold.

FIG. 29. Kaplan-Meier curve analysis of the gene methylation status in urine of castration-resistant prostate cancer (CRPC) cases individually (A-E) and in combinations (F-I) for predicting overall survival/time to death. Urine samples were collected before initiating the 1^{st}-line treatment with abiraterone acetate (AA). Several selected assays are shown. M/U—methylated/unmethylated promoter status. Significant P-values are in bold.

FIG. 30. Kaplan-Meier curve analysis of the biomarker methylation status in urine of castration-resistant prostate cancer (CRPC) cases for predicting overall survival/time-to-death with regard to the duration of responsiveness to hormonal therapy (HT). Urine samples were collected before initiating the 1-line treatment with abiraterone acetate (AA) or after 6±2 months of the treatment. Several selected assays are shown. M/U—methylated/unmethylated promoter status. Significant P-values are in bold.

FIG. 31. Swimmer plot illustrating the derivative methylation estimate xMI3 performance during the monitoring of individual castration-resistant prostate cancer (CRPC) patients undergoing the treatment with abiraterone acetate (AA). Data of ten representative cases are shown. The initiation of the AA therapy is set at 0. xMI3+/xMI3−—the value is predictive/not predictive of adverse pathology.

FIG. 32. Comparison of the biomarker methylation levels in matched urine and plasma samples of four representative individuals (A-D). Methylation of PRKCB, ADAMTS12, NAALAD2, CCDC181, MT1E and APC was analysed. The urine and plasma samples of the same patient were collected at the same type point (before or during the treatment with abiraterone acetate). The connecting lines indicate methylation levels of the same biomarker in urine and plasma samples.

### DEFINITIONS

The following definitions are provided for specific terms which are used in the following and in the claims. Unless defined otherwise, all other scientific and technical terms have the meaning as commonly understood by those of ordinary skill in the art. The terminology that is used herein is not intended to limit the scope of the invention and is used for the purpose to describe particular embodiments only. It is also to be understood that the singular forms a, an and the, as used herein and in the claims, include plural reference unless it is clearly indicated otherwise.

The term “biomarker” as used herein refers to a genomic region that is differentially methylated, wherein the DNA methylation status (incidence) or/and the DNA methylation level indicate the presence or the absence of PCa or/and the condition of a patient diagnosed with PCa and undergoing any kind of treatment strategy, including (but not limited to) active surveillance, watchful waiting, chemotherapy, hormonal therapy, targeted therapy, etc. The qualitative biomarker refers to the DNA methylation status of the particular genomic region, whereas the quantitative biomarker refers to the DNA methylation level of the genomic region. The term “biomarker” might be used interchangeably with “epigenetic biomarker”, “DNA methylation biomarker” or “methylation biomarker”.

The term “primer” as used herein refers to a nucleic acid of at least 16 nucleotides in length which is produced synthetically and, under certain conditions, can hybridize by complementarity to any of the biomarker sequences from the group of SEQ ID NOs: 1-15. The primer can act as a point of initiation of synthesis of a complementary DNA strand.

The term “probe” refers to a primer labelled with one or more tags, which are detectable by measuring fluorescence, and with one or more quencher molecules or the like, i.e. TaqMan®, Molecular Beacon® or Scorpion® probes. In a preferred embodiment, the probes from the group of SEQ ID NO: 46, SEQ ID NO: 49, SEQ ID NO: 52 and SEQ ID NO: 55 are labelled with FAM, JOE or Cy5 at the 5′ end and with BHQ-1, BHQ-3 or TAMRA at the 3′ end. The primers and probes can contain modified nucleotides or nucleotide analogues, which comprise but are not limited to phosphorothioates, 2-′O-alkyl sugar modifications, LNA® and the like.

The term “bisulfite conversion” refers to a method well-known to the person skilled in the art comprising the step of treating DNA with bisulfite or an analogue and thereby converting non-modified (non-methylated, non-hydroxymethylated, etc.) cytosine to uracil, whereas methylated cytosine remains unaffected. Additionally, one or more steps of the purification of the converted DNA can be included. The bisulfite conversion can be performed using a commercially available kit or by performing some part or all of the steps manually.

The terms “DNA methylation” and “methylation” are used herein and in the claims interchangeably and refer to cytosine methylation at C5 position. “Methylated DNA” and “unmethylated DNA” refer to the original (wild-type) methylated or unmethylated DNA template or to the amplified DNA template after bisulfite conversion which was originally methylated or unmethylated.

The terms “DNA methylation status”, “methylation status” and “methylation incidence” are used herein interchangeably and refer to the presence or absence of methylation according to the particular biomarker. The presence of DNA methylation can also be referred to as “DNA hypermethylation” or “hypermethylation”.

The term “DNA methylation level” is interchangeable with the term “methylation level” and refers to the quantity of methylation according to one or more of the biomarkers. The methylation level according to a particular biomarker can be expressed as a relative or absolute value, additionally but not necessarily normalized to a standard or a reference sample (or samples). The value can also be expressed as a percentage or a proportion of a standard sample or a reference sample.

The term “cut-off value” means a specific methylation level above which the results are considered as positive or having a positive methylation status, whereas otherwise the results are classified as negative or having a negative methylation status. Due to the biological variability the cut-off value can vary among different sample types or/and can be dependent on the experimental set-up and/or sample quality. The “cut-off value” can also be referred to as “threshold”.

The term “derivative methylation estimate” (xMI) is a continuous value calculated by combining methylation level and/or methylation status data of at least two of DNA methylation biomarkers. The xMI is based on the biomarker methylation data obtained by means of methylation-specific PCR-based methods. Additionally, the algorithm used to calculate xMI can be modified to include patient's clinical-pathological characteristics and/or other sample's parameters.

The terms “differential methylation”, “differential methylation status” or “differential methylation level” indicate a difference in the methylation status and/or methylation level when comparing two or more samples, groups of samples, biomarkers or genomic loci.

The term “sample” refers to tissue, cancerous or potentially cancerous tissue or cells, preferably from prostate, body fluid (urine, plasma, blood, etc.) or nucleic acids from tissue, cells or body fluid, preferably from an individual being at risk of developing PCa or suspected of having PCa, or a patient diagnosed with PCa. The sample can be obtained from a patient diagnosed with PCa, a diseased patient, a healthy individual or an individual with the unknown state of health.

The terms “individual” and “patient” in some instances are used interchangeably herein and are referred to a human. In a preferred embodiment, the individual is a male human. The patient can have asymptomatic or symptomatic, localized to the prostate, locally advanced or metastatic PCa, i.e. the spectrum of the cancer severity can range from early stage/mild to fatally advanced/extremely severe disease.

The term “progression”, as commonly understood in the field of oncology, refers to changes in characteristics of the disease including adverse changes of clinical-pathological parameters, detection of new cancerous lesions (metastases), development of new symptoms, treatment failure, patient's death and the like.

The terms “biochemical disease recurrence”, “biochemical recurrence” or “biochemical progression” (abbreviated as BCR) refer to the PCa progression defined as an increase of prostate-specific antigen (PSA) concentration in blood or in a fraction of blood (serum, plasma) indicating advancement of the disease. In the context of castration-resistant PCa (CRPC), the term “PSA progression” is used interchangeably.

The term “androgen deprivation therapy” (ADT) as used herein refers to the treatment using hormones or hormone antagonists with the goal to reduce the levels of male hormones, androgens, in the body or to stop them from affecting cancerous cells. ADT can also be referred to as “androgen replacement therapy”, “androgen suppression therapy”, “hormone therapy”, “anti-hormone therapy” or “anti-androgen therapy”.

The term “resistance” refers to the feature of cancer not responding to treatment. Cancer can be resistant to a particular treatment already at the beginning of it (primary resistance), or it can become resistant during the treatment (acquired resistance, also referred to as secondary resistance). The resistance to therapy is commonly understood as unsatisfactory effectiveness of treatment usually resulting in disease progression.

As used herein, a “kit” is a packaged set of reagents and/or tools and/or equipment optionally including instructions for the use of the mentioned set.

### DETAILED DESCRIPTION OF THE INVENTION

The present invention provides the biomarker groups, the methods and the kits useful in detecting or diagnosing PCa, stratifying PCa patients into those having indolent and aggressive form of PCa, predicting PCa progression, performing patient's follow-up, predicting response to a particular therapy, monitoring effectiveness of the treatment or PCa progression when undergoing treatment, and assisting in treatment selection. It is understood that the present invention is not limited to the particular methods and components, described in the Materials and Methods section herein, as these may vary and can be substituted with alternatives. Methodologies of the invention include a step or steps involving comparison of a value or characteristic to a control. The control, as understood herein, is any kind of control or standard sample, characteristic or property familiar to one of ordinary skill in the art useful for comparison purposes. In one embodiment, the control is a value, level, estimate, feature, property, etc., determined in a noncancerous/normal/unaffected sample or sample group, whereas a sample is tissue, cells or body fluid, or DNA from tissue, cells or body fluid obtained from a normal control individual/unaffected individual or an asymptomatic individual. The control exhibits normal/non-pathological traits, features, characteristics, properties, etc., as commonly understood by those having ordinary skill in the art. In another embodiment, the control is a value, level, estimate, feature, property, etc., determined prior to, during or after a particular therapy on a PCa patient at any stage of the disease. In a further embodiment, the control is a predefined value, level, estimate, feature, property, etc. For example, the control can be a predefined methylation level of one or more biomarkers which correlates directly or indirectly to PCa presence, progression potential, etc., to which a patient's sample can be compared.

DNA Methylation Biomarkers and Detection Thereof

The inventors have found genomic loci that are subject to altered DNA methylation in the context of prostate carcinogenesis and tumour development. Cytosines within CpG dinucleotides in the particular genomic loci analysed in test samples are differentially methylated in PCa tissues and noncancerous prostate tissues (NPT). Specifically, the methylation of the genomic loci is more frequent and/or at a higher level in tumours and less common and/or at a lower level in NPT. Furthermore, the differential methylation of particular regions is also observed comparing PCa samples and benign prostatic hyperplasia (BPH) samples. The differences of methylation were found in the genomic loci containing particular genes all of which are known and their detailed descriptions are publicly available in specialized databases, e.g., GeneBank® of the National Institutes of Health (USA). In particular embodiments, the biomarkers include one or more of PRKCB, ADAMTS12, NAALAD2, FILIP1L, ZMIZ1 and KCTD8 (SEQ ID NOs: 1-6 and SEQ ID NOs: 8-10).

Additionally, one or more genes, preferably CCDC181, MT1E, APC and RASSF1 (SEQ ID NO: 7 and SEQ ID NOs: 11-14), can be included in the biomarker panel containing at least one of the biomarkers mentioned previously (i.e. PRKCB, ADAMTS12, NAALAD2, FILIP1L, ZMIZ1 and KCTD8). The DNA methylation biomarkers of the present invention comprise fragments of a polynucleotide sequence that contain CpG dinucleotides and are susceptible to differential methylation. Alternatively, the antisense sequence of the genetic locus containing a biomarker can be utilized. The said antisense biomarker sequence can be analysed with the primers designed easily by a person skilled in the art.

A number of methods can be used to detect, determine, measure, evaluate or characterize the methylation status and/or methylation level of the biomarker or biomarker panel in the context of PCa and, therefore, detect, evaluate, predict the disease, its status, further development and/or response to treatment.

In a preferred embodiment, DNA amplification-based methods (e.g. polymerase chain reaction, PCR) can be used to quantify DNA within a locus flanked by primers. Amplification can be end-point or monitored in real time. Genomic DNA is treated with bisulfite to convert unmodified cytosines to uracils, whereas methylated cytosines remain unaffected, creating an artificial sequence reflecting cytosine modification status in the native DNA. The bisulfite conversion can be performed using commercially available kits or by manual protocols, or combining both. Alternatively, other DNA modifying agents can be used to achieve sequence conversion. Amplification of a DNA sequence of interest is then performed using primers that hybridize to CpG containing loci of a biomarker. In one embodiment, for qualitative evaluation of DNA methylation, two primer pairs, specific to methylated and corresponding unmethylated sequence, are preferably used to amplify the bisulfite-converted DNA. The presence of amplification products with a particular primer pair indicates the methylation status of the sequence of interest. In another embodiment, only primers specific for methylated DNA can be used for the amplification. The amplification product can be detected using DNA intercalating dyes or probes indicating the DNA methylation status.

In one embodiment, the biomarkers can be tested individually, in an uniplex reaction, using one primer pair for the methylated sequence or a set of primers for methylated and unmethylated sequences per locus per test tube. In other embodiments, two or more of the biomarkers can be tested simultaneously in a multiplex reaction using the respective primer pairs or sets. The identification of the amplification products can be achieved by their length analysis and/or using probes, if desired. The amplification can be end-point or monitored in real time.

In the preferred embodiment, DNA methylation status of a biomarker from the group consisting of PRKCB, ADAMTS12, NAALAD2, FILIP1L, ZMIZ1 and KCTD8 indicated by SEQ ID NOs: 1-6 is analysed by means of qualitative end-point methylation-specific PCR (MSP) using a primer set for the methylated and unmethylated sequence assayed in separate test tubes (i.e. generating only one specific amplicon per test tube or none) from the group of oligonucleotides indicated by SEQ ID NOs: 16-39. In another embodiment, two or more biomarkers can be assayed with the respective primer sets performing separate multiplex reactions with primers for methylated and unmethylated biomarker sequences. Optionally, an endogenous control gene may also be analysed with a primer pair specific for bisulfite-converted DNA sequence without CpG (i.e. not influenced by differential methylation), determining the total amount of the converted DNA that may be used to evaluate and/or normalize the sample input. The DNA methylation status is evaluated as methylated or unmethylated according to the presence and/or absence of the specific amplification products with the respective primer pairs for each of the analysed biomarkers. The unmethylated and in vitro methylated controls are used for the test sample comparison and to assess the technical performance of the method. Alternatively, only the methylated control may be included in the experiment if a biomarker is analysed only with the primer pair specific for the methylated sequence.

In other embodiments, DNA methylation status of one or more of the biomarkers from the group consisting of PRKCB, ADAMTS12, NAALAD2, FILIP1L, ZMIZ1 and KCTD8 indicated by SEQ ID NOs: 1-6 can be evaluated in a biomarker panel including at least one of the other DNA methylation biomarkers known in the art, e.g. CCDC181 (SEQ ID NO: 7).

In the preferred embodiment, DNA methylation level of a biomarker from the group of PRKCB, ADAMTS12 and NAALAD2 indicated by SEQ ID NOs: 8-10 is analysed by means of quantitative real-time methylation-specific PCR (QMSP) using a primer pair and a probe all specific for the methylated sequence. Alternatively, at least one of the oligonucleotides from the primer pair and probe set may be specific for the methylated sequence. In another embodiment, the DNA methylation level of a biomarker can be analysed using primers and/or probes specific for the unmethylated biomarker sequence with or without analysing the methylated biomarker sequence. Simultaneously, an endogenous control gene, preferably ACTB (SEQ ID NO: 15), is analysed to normalize for the sample input. Other endogenous control genes used in the related art can be included as substitutes of ACTB. The biomarker and the endogenous control gene can be analysed in uniplex reactions or two or more biomarkers and/or the endogenous control gene can be assayed with the respective primer pairs and probes in a full or partial multiplex reaction. The probes are detectably labelled including but not limited to TaqMan or Molecular Beacon probes. Preferably, the TaqMan hydrolysis probes labelled with FAM, JOE, HEX, VIC, Cy5, Cy3, etc. at 5′-end and a compatible quencher moiety at 3′-end, e.g. BHQ1-3, TAMRA, etc., are used for the biomarker assays. The in vitro methylated control is preferably included as the methylated DNA standard and used as a reference sample to determine the methylation level of a biomarker. Alternatively, the biomarker analysis may be performed without including the methylated control and, thus, determining the methylation level normalized only to the endogenous control gene. In some embodiments, a passive-fluorescence dye, also referred to as a passive dye, reference dye, reference fluorophore, etc., can be included in the biomarker assay to account partially for technical variability. In the preferred embodiment, the passive-fluorescence dye is rhodamine X (ROX).

In one embodiment, the DNA methylation level of a biomarker from the group consisting of PRKCB, ADAMTS12 and NAALAD2 indicated by SEQ ID NOs: 8-10 is determined by the cycle of quantification value (Cq) in the QMSP assay. Optionally, the DNA methylation level may be estimated by evaluating the fluorescence signal intensity at a particular cycle of the QMSP reaction. The Cq value of a biomarker in a sample is used to calculate the relative DNA methylation level preferably, but not necessarily normalized according to the parameters including the methylated control, endogenous control gene and passive-fluorescence dye. The DNA methylation level of a biomarker in a sample can be expressed as a proportion or a percentage of the DNA methylation level of the biomarker in the methylated control.

In another embodiment, the DNA methylation status of a biomarker from the group consisting of PRKCB, ADAMTS12 and NAALAD2 indicated by SEQ ID NOs: 8-10 can be determined from the DNA methylation level. The biomarker in the sample can be considered as methylated if the determined methylation level is more than or not less than a cut-off value (also referred to as a threshold). Otherwise, the biomarker in a sample is considered unmethylated. The cut-off value can be predefined, set automatically by the analysis software or determined empirically. In the preferred embodiment, the cut-off value is selected based on the average or median DNA methylation level of the biomarker in control (normal, healthy) samples. In the more preferred embodiment, the cut-off value is the average methylation level of the biomarker in BPH samples or is empirically set at 0.1% for any of the biomarkers.

In another embodiment, the DNA methylation of any two or all three biomarkers from the group consisting of PRKCB, ADAMTS12 and NAALAD2 indicated by SEQ ID NOs: 8-10 can be defined as a derivative methylation estimate, referred to as xMI, including both the DNA methylation level and the DNA methylation status of the included biomarkers and determined according to the algorithm provided in Formula 1. In the preferred embodiment, the xMI is determined according to the combination of the three biomarkers including PRKCB, ADAMTS12 and NAALAD (SEQ ID NOs: 8-10) assayed using the primers and probes according to SEQ ID NOs: 44-52.

$$xMI = {K}_{y} \times A \times N \times {\sum }_{i = 1}^{N} Xi / ( 1 + {BΣ}_{Xi < c}^{N} 1 )$$

Formula 1. The algorithm used for calculating the derivative methylation estimate xMI for a combination of at least two DNA methylation biomarkers. X—the relative methylation level of a particular gene, N—the number of genes included in a panel, A and B—empirically determined coefficients, c—the cut-off value for the qualitative interpretation of the DNA methylation level of a biomarker, K_{Y}—the optional correction coefficient that is a quantitative estimate accounting for a particular clinical-pathological patient's parameter or sample's property y.

In some embodiments, the DNA methylation of at least one other biomarker, preferably from the group of biomarkers consisting of CCDC181, MT1E, APC and RASSF1 indicated by SEQ ID NOs: 11-14, can be included in determining the xMI together with at least one of the biomarkers of the present invention (i.e. PRKCB, ADAMTS12 and NAALAD2 indicated by SEQ ID NOs: 8-10). In the preferred embodiment, the xMI is determined according to the combination of the four biomarkers including PRKCB, ADAMTS12, NAALAD (SEQ ID NOs: 8-10) and CCDC181 (SEQ ID NO: 11) assayed using the primers and probes according to SEQ ID NOs: 44-52 and SEQ ID NOs: 53-55. Generally, the algorithm provided in Formula 1 may be applied to any combination of up to 20 quantitative DNA methylation biomarkers known in the related art, with or without the biomarkers of the present invention.

In other embodiments, other target-specific amplification methods that are alternative to PCR may be used for the biomarker analysis. Such methods include but are not limited to selective amplification of target polynucleotide sequences, strand displacement technology, nick displacement amplification.

Additional methods for detecting the DNA methylation biomarkers of the present invention can involve genomic or gene-targeted sequencing with or without a step of DNA treatment with bisulfite. In some embodiments, digestion of the biomarker amplicons using restriction enzymes can be included in the methodology. Other multiple techniques for the analysis of DNA methylation are known in the art which comprise without limitation MLPA, HeavyMethyl, ConLight-MSP, COBRA, MS-SNuPE, MS-SSCA, MassARRAY, oligonucleotide-based microarray platforms, pyrosequencing, etc., as discussed for instance in Kurdyukov and Bullock (2016) [17].

Kits for the Detection of Prostate Cancer Biomarkers

The present invention provides kits for testing DNA methylation biomarkers in the context of PCa. The kits are used to detect, measure or estimate the methylation status and/or methylation level of the biomarkers described herein. The kits can comprise at least one primer or probe or at least one polynucleotide that hybridizes to at least one of the biomarker sequences. The kits also comprise at least of the reagents or components for detecting biomarker methylation. The reagents can include but are not limited to sodium bisulfite, methylation-dependent or methylation-sensitive restriction enzymes, methylation-specific antibody or methylcytosine-binding moiety. The reagents for the detection of methylation can modify, cut or interact with the sequence that is the product of the biomarker. A methylcytosine-binding moiety refers to a molecule that specifically binds to methylcytosine (e.g. antibodies, methyl-binding domains or proteins containing such domains, restriction enzymes lacking nuclease activity but retaining methylated-DNA binding activity). The kits may further comprise detectable labels, barcode oligonucleotides, etc., linked to the polynucleotide of the biomarker. The kits may also include DNA polymerase or other PCR reagents, test tubes, plates, pipettes and other components used in performing the assays. The kits may also include written instructions, protocols, recommendations for the use of at least one of any of the components in the kits.

In some embodiments, the kits comprise one or more of polynucleotides specifically amplifying at least a fragment of a genomic locus of a biomarker of the invention including, but not limited to PRKCB, ADAMTS12, NAALAD2, FILIP1L, ZMIZ1 and KCTD8 indicated by SEQ ID NOs: 1-6 and SEQ ID NOs: 8-10. Additionally, the kit can also comprise at least a fragment of a genomic locus of a known biomarker including, but not limited to CCDC181, MT1E, APC and RASSF1, preferably indicated by SEQ ID NO: 7 and SEQ ID NOs: 11-15.

In some embodiments, the kits comprise one or more of the primers or probes indicated by SEQ ID NOs: 16-39 and SEQ ID NOs: 44-52 specifically amplifying or at least hybridizing to one or more of the biomarkers or their fragments including PRKCB, ADAMTS12, NAALAD2, FILIP1L, ZMIZ1 and KCTD8. Optionally, one or more of the primers and probes indicated by SEQ ID NOs: 40-43 and SEQ ID NOs: 53-67 can be included in the kits together with at least one of the above (SEQ ID NOs: 16-39 and SEQ ID NOs: 44-52). In some embodiments, the kits can also include sodium bisulfite together with one or more of the primers and probes identified by SEQ ID NOs: 16-39 and SEQ ID NOs: 44-52.

In some embodiments, the kits can comprise methylation-dependent or methylation-sensitive restriction enzymes or methylcytosine-binding moiety, primers or probes for whole-genome amplification, and at least one of the polynucleotides and/or primers and/or probes indicated by SEQ ID NOs: 1-6, SEQ ID NOs: 8-10, SEQ ID NOs: 16-39 and SEQ ID NOs: 44-52 to analyse at least one of the biomarkers including, but not limited to PRKCB, ADAMTS12, NAALAD2, FILIP1L, ZMIZ1 and KCTD8. Optionally, one or more of the primers and probes indicated by SEQ ID NOs: 40-43 and SEQ ID NOs: 53-67 can be included in the kits together with at least one of the above (SEQ ID NOs: 16-39 and SEQ ID NOs: 44-52).

In some embodiments, methods for detecting methylation can include cutting DNA with a methylation-dependent or methylation-sensitive restriction enzyme (endonuclease) with subsequent analysis of the cut and/or uncut DNA. A method can include amplification step of cut and/or uncut DNA after digestion with restriction enzymes, or intact DNA before digestion. Amplification can be achieved with gene-specific or random primers. Additionally, adaptors of various kind can be added to the fragments of DNA and amplification can be performed with primers that hybridize to the adaptor oligonucleotides.

Biomarkers for PCa Detection and Diagnosis

The invention provides the diagnostic tools or means to determine PCa. The biomarkers identified in the present invention show differential methylation among histologically different prostate tissue samples and therefore are useful in determining the PCa status. In particular embodiments, the biomarkers can be measured utilizing the methods described herein and compared/associated to the PCa status. More specifically, the biomarkers can be used in diagnostic tests to determine, qualify, assess or characterize PCa, e.g. to diagnose, to predict PCa in an individual, subject or patient or to evaluate the disease severity at the time of detection or diagnosis. More specifically, the methylation status of PRKCB, ADAMTS12, NAALAD2, FILIP1L, ZMIZ1 and KCTD8 can be used as diagnostic biomarkers of PCa individually or in various combinations as a biomarker panel, with or without other biomarkers, e.g. CCDC181. In particular embodiments, the biomarkers include one or more of PRKCB (SEQ ID NO: 1), ADAMTS12 (SEQ ID NO: 2), NAALAD2 (SEQ ID NO: 3), FILIP1L (SEQ ID NO: 4), ZMIZ1 (SEQ ID NO: 5) and KCTD8 (SEQ ID NO: 6). In more specific embodiments, the particular biomarker can comprise a fragment of a polynucleotide according to SEQ ID NOs: 1-6 wherein the fragment comprises not less than 90% of consecutive nucleotides. Additionally, the biomarkers can include one or more known biomarkers, preferably CCDC181 (SEQ ID NO: 7). In another embodiment, the biomarkers include one or more of PRKCB (SEQ ID NO: 8), ADAMTS12 (SEQ ID NO: 9) and NAALAD2 (SEQ ID NO: 10). In more specific embodiments, the particular biomarker can comprise a fragment of a polynucleotide according to SEQ ID NOs: 8-10 wherein the fragment comprises not less than 90% of consecutive nucleotides. Additionally, the biomarkers can include one or more known biomarkers, preferably from the group consisting of CCDC181 (SEQ ID NO: 11), MT1E (SEQ ID NO: 12), APC (SEQ ID NO: 13) and RASSF1 (SEQ ID NO: 14).

A method for identifying PCa in a subject can comprise the steps of: a) obtaining a biological sample from the subject; b) determining the methylation status of one or more biomarkers of this invention in the test sample; c) identifying the methylation status of one or more biomarkers from the group of PRKCB, ADAMTS12, NAALAD2, FILIP1L, ZMIZ1 and KCTD8 according to SEQ ID NOs: 1-6, or d) identifying the methylation status of one or more biomarkers from the group of PRKCB, ADAMTS12 and NAALAD2 according to SEQ ID NOs: 8-10, wherein the methylation status of said biomarker(-s) is indicative of the PCa presence or stage/grade of PCa or increased risk of PCa development. In one embodiment, the DNA methylation status of the biomarker for PCa diagnostics can be analysed by uniplex or multiplex MSP in a sample of prostate tissue, body fluid, preferably urine, or cells, or DNA sample from prostate tissue, body fluid or cells. In another embodiment, the DNA methylation status of the biomarker can be analysed by uniplex or multiplex QMSP in a sample of prostate tissue, body fluid, preferably urine, or cells, or DNA sample from prostate tissue, body fluid or cells.

The power of a diagnostic test to correctly identify status can be measured by calculating the assay selectivity parameters, most commonly including the sensitivity, specificity, accuracy, and by estimating the area under the Receiver operating characteristic (ROC) curve (AUC). The sensitivity is defined as the percentage of true positives that are predicted by a test as positives, whereas the specificity is the percentage of true negatives that are predicted as negatives. The accuracy is the percentage of true positives and true negatives relative to all tested samples. A ROC curve provides the sensitivity as a function of [100%−specificity], whereas the larger AUC indicates the more powerful predictive value of a test. By adjusting the diagnostic cut-off used in the assay, one can increase the sensitivity and/or specificity of the diagnostic assay/test as preferred by a person performing the diagnostic test, as is well understood in the art. There are many ways to interpret the methylation status of two or more biomarkers for the diagnostic purposes. For instance, the methylation status of a set of biomarkers can be assumed as the methylation status of at least one, two, three, etc. biomarkers in the panel. In certain embodiments, the values of the methylation status of the biomarkers may be combined by any appropriate mathematical method (e.g. discriminant functional analysis, generalized linear models, etc.). It is well understood that a skilled artisan will be able to select easily the appropriate method for evaluating the methylation status of the biomarker combination of the present invention.

Biomarkers for PCa Severity, Prognosis and Follow-Up

In other embodiments, the invention provides tools and methods for determining PCa severity and the predict the risk of PCa progression in a patient at the time of diagnosis or at any time after the diagnosis, as well as for the patient's follow-up (by using body fluid samples), as the methylation of the biomarkers changes over time. The methylation status and the higher methylation levels of the biomarkers relate to the adverse PCa pathology and progression, whereas the unmethylated status and lower or absent methylation levels are associate to the disease remission/improvement. In some embodiments, the PCa progression can be defined as BCR.

The severity of PCa and the risk of BCR can be determined by identifying the methylation status of one or more biomarkers from the group consisting of PRKCB, ADAMTS12, NAALAD2, FILIP1L, ZMIZ1 and KCTD8 according to SEQ ID NOs: 1-6 and SEQ ID NOs: 8-10. Additionally, the biomarkers can include one or more known biomarkers, preferably from the group consisting of CCDC181 (SEQ ID NO: 7 or SEQ ID NO: 11), MT1E (SEQ ID NO: 12), APC (SEQ ID NO: 13) and RASSF1 (SEQ ID NO: 14). In the preferred embodiment, the methylation status of the biomarkers is evaluated in prostate tissue sample or cells, or DNA sample obtained from prostate tissue or cells. The methylation status of at least one of the biomarkers in the biomarker panel can be assumed as the methylation status of that panel. For instance, the biomarker panels for determining the methylation status can consist of: a) PRKCB and ADAMTS12; b) PRKCB and NAALAD2; c) PRKCB, ADAMTS12 and NAALAD2; d) PRKCB and CCDC181; e) PRKCB, NAALAD2 and CCDC181; f) PRKCB, ADAMTS12 and FILIP1L; g) PRKCB, ADAMTS12, NAALAD2 and CCDC181; i) PRKCB, ADAMTS and MT1E; etc.

In another preferred embodiment, the severity of PCa and the risk of BCR can be determined by identifying the methylation level of one or more biomarkers from the group consisting of PRKCB, ADAMTS12 and NAALAD2 according to SEQ ID NOs: 8-10. Additionally, the biomarkers can include one or more known biomarkers, preferably from the group consisting of CCDC181 (SEQ ID NO: 11), MT1E (SEQ ID NO: 12), APC (SEQ ID NO: 13) and RASSF1 (SEQ ID NO: 14). In the preferred embodiment, the methylation levels of the biomarkers are evaluated in body fluid samples or DNA samples obtained from body fluid samples, preferably urine, from PCa patients. In other embodiments, the methylation levels can be evaluated in prostate tissue sample or cells, or DNA sample obtained from prostate tissue or cells. The methylation levels of the biomarkers can be interpreted individually or combined by using any appropriate mathematical method. In the preferred embodiment, the xMI values are used for the biomarker panels when evaluating PCa severity and predicting the disease progression. For instance, the biomarker panels for determining the methylation levels can consist of: a) PRKCB, ADAMTS12 and NAALAD2; b) PRKCB, ADAMTS12, NAALAD2 and CCDC181; c) PRKCB, ADAMTS12 and MT1E; d) PRKCB and ADAMTS12; e) PRKCB and NAALAD2; etc.

The methylation status or the methylation levels of the biomarker or the biomarker panel can be combined with the TMPRSS2-ERG fusion transcript status, well-known as the PCa-specific molecular alteration in the related art. In another embodiment, the methylation status or methylation levels of the biomarker or the biomarker panel can be combined with PSA or patient's clinical-pathological characteristics, such as the stage of the disease or the differentiation grade of the primary tumour tissue at the diagnosis, for the improved prognostic performance.

A method for determining PCa severity and/or predicting the disease progression in a subject can comprise the steps of: a) obtaining a biological sample from the subject; b) determining the methylation status and/or methylation levels of one or more biomarkers of this invention in the test sample; c) identifying the methylation status of one or more biomarkers from the group of PRKCB, ADAMTS12, NAALAD2, FILIP1L, ZMIZ1 and KCTD8 according to SEQ ID NOs: 1-6, or d) identifying the methylation status of one or more biomarkers from the group of PRKCB, ADAMTS12 and NAALAD2 according to SEQ ID NOs: 8-10; or e) identifying the methylation levels of one or more biomarkers from the group of PRKCB, ADAMTS12 and NAALAD2 according to SEQ ID NOs: 8-10; wherein the methylation status of said biomarker(-s) is indicative of the risk of PCa progression. In one embodiment, the methylation status of the biomarker for PCa prognosis can be analysed by uniplex or multiplex MSP in a sample of prostate tissue, body fluid, preferably urine, or cells, or DNA sample from prostate tissue, body fluid or cells. In another embodiment, the methylation levels or methylation status or the of the biomarker can be analysed by uniplex or multiplex QMSP in a sample of prostate tissue, body fluid, preferably urine, or cells, or DNA sample from prostate tissue, body fluid or cells.

Biomarkers for Castration-Resistant Prostate Cancer Patient Management/Monitoring

In certain embodiments, the invention provides tools and methods for PCa patient management/monitoring, more specifically for CRPC cases. Such management is understood as the subsequent actions of a clinician after determining or predicting adverse course of the disease using the tools and methods of the present invention. In certain embodiments, the PCa/CRPC assessment utilizing the present invention can indicate the need to change, discontinue, initiate, modify, etc., a particular therapy. Alternatively, a result indicating low progression risk can suggest low necessity of the new treatment and/or can be followed by the continuation of the undergoing therapy.

The patient's monitoring can be pursued by identifying the methylation levels of one or more biomarkers from the group consisting of PRKCB, ADAMTS12 and NAALAD2 according to SEQ ID NOs: 8-10. Additionally, the biomarkers can include one or more known biomarkers, preferably from the group consisting of CCDC181 (SEQ ID NO: 11), MT1E (SEQ ID NO: 12), APC (SEQ ID NO: 13) and RASSF1 (SEQ ID NO: 14). In the preferred embodiment, the methylation levels of the biomarkers are evaluated in body fluid samples or DNA samples obtained from body fluid samples, preferably urine or plasma, from CRPC patients, since samples of prostate tissue and/or metastatic sites are usually unobtainable due to the disease severity and patients' overall health status. The methylation levels of the biomarkers can be interpreted individually or combined by using any appropriate mathematical method. In the preferred embodiment, the xMI values are used for the biomarker panels when evaluating CRPC patient's status and predicting the disease progression. In certain embodiments, the differences of the methylation levels, xMI values or any other derivative estimates obtained from the biomarker results can be compared between the serial samples of a particular CRPC patient. The higher methylation levels, as compared to any kind of control/standard/baseline samples, or the increasing methylation indices are associated with the CRPC progression/developing of new symptoms/worsening of the general health state and, thus, can be used to identify the ongoing pathological process and to consider modification in the treatment regimen. The methylation status of one or more biomarkers of the group consisting of PRKCB, ADAMTS12 and NAALAD2 according to SEQ ID NOs: 8-10 can be used for the CRPC patient's monitoring purposes. Optionally, the methylation status of one or more known biomarkers, preferably CCDC181 (SEQ ID NO: 11), MT1E (SEQ ID NO: 12), APC (SEQ ID NO: 13) and RASSF1 (SEQ ID NO: 14), can be included in the biomarker panel together with at least one of the above mentioned biomarkers of the present invention (SEQ ID NOs: 8-10). The methylation status of at least one of the biomarkers in the biomarker panel can be assumed as the methylation status of that panel. For instance, the biomarker panels for determining the methylation levels or methylation status can consist of: a) PRKCB, ADAMTS12 and NAALAD2; b) PRKCB, ADAMTS12, NAALAD2 and CCDC181; c) PRKCB, ADAMTS12 and MT1E; d) PRKCB and ADAMTS12; e) PRKCB and NAALAD2; etc.

In another embodiment, the methylation levels or the methylation status of the biomarkers can be combined with the PSA or patient's clinical-pathological characteristics, such as time from PCa diagnosis to CRPC development, the duration of androgen deprivation therapy (ADT), the type of local treatment, etc., for the improved prognostic performance.

A method for determining PCa severity and/or predicting the disease progression in a subject can comprise the steps of: a) obtaining a biological sample, preferably urine, from the subject; b) determining the methylation level and/or methylation status of one or more biomarkers of this invention; c) identifying the methylation levels of one or more biomarkers from the group of PRKCB, ADAMTS12 and NAALAD2 according to SEQ ID NOs: 8-10; or d) identifying the methylation status of one or more biomarkers from the group of PRKCB, ADAMTS12 and NAALAD2 according to SEQ ID NOs: 8-10; wherein the methylation levels and/or the methylation status of said biomarker(-s) are indicative of the CRPC progression. In one embodiment, the methylation levels or methylation status can be analysed by uniplex or multiplex QMSP in a body fluid sample, preferably urine or plasma, or a DNA sample from body fluid obtained from a CRPC patient.

Biomarkers for Determining Treatment Efficacy

In another embodiment, the present invention provides tools and methods for determining the therapeutic efficacy of a pharmaceutical drug. Such tools can be useful for predicting the response to a drug, e.g. abiraterone acetate (AA), before initiating the treatment and/or monitoring the progress of the CRPC patient undergoing the treatment with the drug. Furthermore, the biomarkers of the present invention are useful for the patients' monitoring in performing clinical trials of the drug. If the drug impacts the condition of the patient, the methylation levels and/or methylation status of the discovered biomarkers change over time in direct association between the methylation and adverse pathology/progression. The therapeutic efficacy can be evaluated using the patient's monitoring data, e.g. time to PSA progression, overall progression/radiologic progression or patient's death.

In certain embodiments, the methylation levels or methylation status of one or more biomarkers from the group consisting of PRKCB, ADAMTS12 and NAALAD2 indicated by SEQ ID NOs: 8-10 can be evaluated in a single sample or in a sample series collected from the CRPC patient at different time points before and/or during a course of the treatment. Additionally, the biomarkers can include one or more known biomarkers, preferably from the group consisting of CCDC181 (SEQ ID NO: 7 or SEQ ID NO: 11), MT1E (SEQ ID NO: 12), APC (SEQ ID NO: 13) and RASSF1 (SEQ ID NO: 14). In the preferred embodiment, the methylation levels of the biomarkers are evaluated in body fluid samples or DNA samples obtained from body fluid samples, preferably urine or plasma. The methylation levels of the biomarkers can be interpreted individually or combined by using any appropriate mathematical method. In the preferred embodiment, the xMI values are used for the biomarker panels when evaluating the therapeutic efficacy and the development of treatment resistance. The higher methylation levels, as compared to any kind of control/standard/baseline/previous samples, or the increasing methylation indices are associated with the presence or development of treatment resistance and can be used to identify the ongoing pathological process and to consider modification in the treatment regimen or change to another drug. The methylation status of one or more biomarkers of the group consisting of PRKCB, ADAMTS12 and NAALAD2 according to SEQ ID NOs: 8-10 can be used to evaluate the treatment efficacy. Optionally, the methylation status of one or more known biomarkers, preferably CCDC181 (SEQ ID NO: 11), MT1E (SEQ ID NO: 12), APC (SEQ ID NO: 13) and RASSF1 (SEQ ID NO: 14), can be included in the biomarker panel together with at least one of the above mentioned biomarkers of the present invention (SEQ ID NOs: 8-10). The methylation status of at least one of the biomarkers in the biomarker panel can be assumed as the methylation status of that panel. For instance, the biomarker panels for determining the methylation levels or methylation status can consist of: a) PRKCB, ADAMTS12 and NAALAD2; b) PRKCB, ADAMTS12, NAALAD2 and CCDC181; c) PRKCB, ADAMTS12 and MT1E; d) PRKCB and ADAMTS12; e) PRKCB and NAALAD2; etc.

In another embodiment, the methylation levels or the methylation status of the biomarkers can be combined with the PSA or patient's clinical-pathological characteristics, such as time from PCa diagnosis to CRPC development, the positive response duration of the previously administered drug, the duration of ADT, radiographic imaging data, etc., for improved predictive performance.

A method for determining the treatment efficacy in a CRPC patient can comprise the steps of: a) obtaining a biological sample, preferably urine, from the subject; b) determining the methylation level and/or methylation status of one or more biomarkers of this invention; c) identifying the methylation levels of one or more biomarkers from the group of PRKCB, ADAMTS12 and NAALAD2 according to SEQ ID NOs: 8-10; or d) identifying the methylation status of one or more biomarkers from the group of PRKCB, ADAMTS12 and NAALAD2 according to SEQ ID NOs: 8-10; wherein the methylation levels and/or the methylation status of said biomarker(-s) are indicative of the treatment effectiveness/positive response or the presence or development of resistance. The methylation levels or methylation status can be analysed by uniplex or multiplex QMSP in a body fluid sample, preferably urine or plasma, or a DNA sample from body fluid obtained from a CRPC patient.

Collectively, the present invention involves the tools, methods and kits for PCa evaluation at the various stages of the disease and provides a set of the methylation biomarkers unique for their wide applicability as diagnostic, as well as prognostic and predictive biomarkers in the context of PCa/CRPC.

### EXAMPLES

Materials and Methods

Localized and Locally Advanced PCa Cases

Fresh-frozen prostate tissue samples from patients diagnosed with localized or locally advanced prostate adenocarcinoma were collected between 2008 and 2014 from 151 patients who underwent radical prostatectomy (RP) at the Urology Centre of Vilnius University Hospital “Santaros Klinikos”. Noncancerous prostate tissue (NPT) samples were available from 51 PCa patients. As a control group, 17 BPH samples, obtained from open prostatectomy material, were included in the study. These samples were used to identify biomarkers distinguishing tumour from benign tissue and aggressive PCa cases form indolent cases. All tissues were sampled and evaluated by an expert pathologist as reported [18].

Voided urine samples were collected from 152 patients diagnosed with localized or locally advanced PCa, and from 30 asymptomatic cases (ASC). For comparison, catheterized urine samples (collected during prostatectomy) from 29 BPH patients were also included. All urine samples (˜30 ml) were centrifuged at 1000 rpm for 15 min at 4° C. (Hettich® Universal 320R Centrifuge, DJB Labcare, Buckinghamshire, United Kingdom), supernatant was removed, and sediments were washed twice with 1×PBS. Samples were stored at <−70° C. until use.

None of these patients had preoperatively received hormone therapy, chemotherapy, or radiotherapy. Following the EAU-ESTRO-SIOG Guidelines on Prostate Cancer 2016, BCR was defined by two consecutive increases ≥0.2 ng/mL in serum PSA level after RP [8]. Patient and sample data are provided in Table 1.

TABLE 1
[Clinical-pathological and molecular characteristics of the patients with localized or locally advanced prostate cancer according to the analysis groups.]

| | Methylation analysis group- | | | Gene expression | | | | |
|---|---|---|---|---|---|---|---|---|
| Parameter | tissue | | | analysis group¹ | | Methylation analysis group-urine | | |
| Group | PCa | NPT | BPH | PCa | NPT | PCa | BPH | ASC |
| composition | (N = 151) | (N = 51) | (N = 17) | (N = 81) | (N = 25) | (N = 152) | (N = 29) | (N = 33) |

Age, years

| | | | | | | | | |
|---|---|---|---|---|---|---|---|---|
| Mean ± SD | 61 ± 8 | 62 ± 7 | 70 ± 8 | 61 ± 8 | 61 ± 6 | 62 ± 8 | 73 ± 7 | 61 ± 8 |
| (range) | [41; 82] | [46; 74] | [59; 80] | [41; 82] | [48; 74] | [41; 76] | [58; 83] | [40; 73] |

Tumour stage, N

| | | | | | | | | |
|---|---|---|---|---|---|---|---|---|
| ≤pT2 | 96 | — | — | 51 | — | 99 | — | — |
| ≥pT3 | 55 | — | — | 30 | — | 44 | — | — |
| Unknown | 0 | — | — | 0 | — | 9 | — | — |

ISUP grade group (Gleason score), N

| | | | | | | | | |
|---|---|---|---|---|---|---|---|---|
| I (3 + 3) | 34 | — | — | 13 | — | 30 | — | — |
| II (3 + 4) | 84 | — | — | 48 | — | 86 | — | — |
| III (4 + 3) | 23 | — | — | 14 | — | 21 | — | — |
| IV (8) | 3 | — | — | 2 | — | 1 | — | — |
| V (9) | 5 | — | — | 3 | — | 5 | — | — |
| Unknown | 2 | — | — | 1 | — | 9 | — | — |

Tumour cellularity, N

| | | | | | | | | |
|---|---|---|---|---|---|---|---|---|
| 90-100% | 73 | — | — | 48 | — | — | — | — |
| 70-89% | 38 | — | — | 24 | — | — | — | — |
| 50-69% | 40 | — | — | 8 | — | — | — | — |
| 1-3% | — | 3 | — | — | 0 | — | — | — |
| 0% | — | 48 | — | — | 25 | — | — | — |

BCR status, N

| | | | | | | | | |
|---|---|---|---|---|---|---|---|---|
| Yes (mean | 35 (18) | 16 (16) | — | 30 (19) | 7 (20) | 24 (18) | — | — |
| time to BCR, | | | | | | | | |
| mo.) | | | | | | | | |
| No (mean | 99 (38) | 32 (47) | — | 49 (42) | 17 (58) | 91 (26) | — | — |
| follow-up, | | | | | | | | |
| mo.) | | | | | | | | |
| Unknown | 17 | 3 | — | 2 | 1 | 37 | — | — |

PSA, ng/mL

| | | | | | | | | |
|---|---|---|---|---|---|---|---|---|
| Mean ± SD | 10.6 ± 11.1 | 9.4 ± 8.6 | 7.3 ± 6.6 | 10.8 ± 9.4 | 10.0 ± 9.5 | 8.9 ± 10.0 | 9.3 ± 13.4 | — |
| (range) | [2.5; 84.2] | [2.5; 44.0] | [0.8; 28.1] | [2.5; 44.0] | [2.5; 44.0] | [2.5; 98.7] | [0.8; 69.8] | |
| Unknown | 2 | 1 | 0 | 2 | 1 | 0 | 2 | — |

Prostate mass, g

| | | | | | | | | |
|---|---|---|---|---|---|---|---|---|
| Mean ± SD | 48 ± 17 | 50 ± 20 | — | 48 ± 17 | 48 ± 20 | 51 ± 19 | — | — |
| (range) | [16; 123] | [16; 104] | | [16; 123] | [16; 104] | [20; 117] | | |
| Unknown | 0 | 0 | — | 0 | 0 | 1 | — | — |

TMPRSS2-ERG fusion transcript, N

| | | | | | | | | |
|---|---|---|---|---|---|---|---|---|
| Yes | 77 | — | — | 46 | — | 14 | — | — |
| No | 44 | — | — | 26 | — | 10 | — | — |
| Unknown | 8 | — | — | 9 | — | 128 | — | — |

PCa—prostate cancer,
NPT—noncancerous prostate tissue,
BPH—benign prostatic hyperplasia,
ASC—asymptomatic cases,
ISUP—International Society of Urological Pathology,
BCR—biochemical disease recurrence,
PSA—prostate-specific antigen,
SD—standard deviation.

Castration-Resistant PCa Cases

In this prospective part of the study, patients were enrolled prior to the initiation of AA therapy after developing CRPC on ADT or during the AA therapy (N=103 and N=127 samples, respectively; N=136 patients in total). The subjects were included between May 2016 and July 2018, and treated at the National Cancer Institute (Vilnius, Lithuania). Clinical data were extracted from patients' records by staff clinicians. One, two and three urine samples were collected at different time points with regard to the treatment.

The prognostic and predictive potential of the biomarkers was evaluated by analysing the patients' monitoring data: overall survival (time to death), progression-free survival, primary and acquired resistance to AA, absence of PSA reduction by >50% (PSA progression). Overall survival and progression-free survival were calculated from the date of sample collection to the event. Primary resistance has been defined as a treatment failure within the first 3 months after treatment initiation, as a result of overt clinical progression, with or without imaging processing. Treatment failure that occurred later was considered as acquired resistance [19]. Patients characteristics are summarized in Table 2.

TABLE 2
[Clinical-pathological characteristics of the patients with castration-resistant prostate cancer (CRPC).]

| | | Samples available for analysis (N = 230) | |
|---|---|---|---|
| | All patients | Before AA/baseline | During AA treatment |
| Parameter | (N = 136) | (N = 103) | (N = 127) |
| Age at diagnosis, yr. | 66/66 [49; 84] | 67/67 [50; 84] | 65/64 [49; 84] |
| (mean/median; interval) | | | |
| Age at last follow-up/death, | 75/75 [56; 91] | 75/76 [56; 91] | 75/74 [56; 91] |
| yr. (mean/median; interval) | | | |

ISUP group (Gleason score; biopsy), N

| | | | |
|---|---|---|---|
| 1 (3 + 3) | 53 | 40 | 52 |
| 2 (3 + 4) | 22 | 17 | 20 |
| 3 (4 + 3) | 18 | 13 | 16 |
| 4 (8) | 22 | 15 | 20 |
| 5 (9 or 10) | 12 | 9 | 10 |
| Unknown | 9 | 9 | 9 |

Primary staging, N

| | | | |
|---|---|---|---|
| Localized (≤cT2) | 48 | 35 | 46 |
| Locally advanced (cT3) | 73 | 53 | 71 |
| Metastatic (cT4) | 11 | 11 | 7 |
| Unknown | 4 | 4 | 3 |

Type of local treatment, N

| | | | |
|---|---|---|---|
| Radical prostatectomy | 12 | 6 | 13 |
| Radiation therapy | 48 | 34 | 53 |
| None | 75 | 62 | 60 |
| Unknown | 1 | 1 | 1 |
| ADT duration, mo. | 53/39 [0; 159] | 54/40.5 [0; 159] | 55/41 [0; 159] |
| (mean/median; interval) | | | |
| Unknown, N | 4 | 3 | 4 |

Prior chemotherapy, N

| | | | |
|---|---|---|---|
| Docetaxel | 34 | 16 | 38 |
| None | 102 | 87 | 89 |
| PSA level at diagnosis, ng/ml | 399.6/19.4 [3.2; >1000] | 162.7/20.5 [3.2; >1000] | 418/19.9 [3.2; >1000] |
| (mean/median; interval) | | | |
| Unknown, N | 124 | 11 | 11 |

Baseline PSA level, ng/ml*

| | | | |
|---|---|---|---|
| Before 1^{st}-line AA | 117.4/36.5 [0.4; 1563] | 134.0/38.2 [2.1; 1563.0] | 92.2/28.5 [2.1; 1563.0] |
| Before 2^{nd}-line AA | 79.7/40.3 [1.4; 760.4] | 103.2/36.9 [3.7; 760.4] | 69.8/45.2 [1.4; 668.7] |

Resistance to AA, N

| | | | |
|---|---|---|---|
| Primary | 20 | 17 | 9 |
| Acquired | 70 | 47 | 74 |
| None | 40 | 33 | 41 |
| Unknown | 6 | 6 | 3 |

Overall survival status, N***

| | | | |
|---|---|---|---|
| Dead | 44 | 35 | 27 |
| Alive | 92 | 68 | 100 |

*At the time of initiating/changing treatment;
**time from the treatment initiation;
***at the time of last follow-up.
PCa—prostate cancer,
AA—abiraterone acetate,
ADT—androgen deprivation therapy,
PSA—prostate-specific antigen.

Genome-Wide DNA Methylation Profiling

For the screening step, genome-wide DNA methylation profiling data of 9 paired PCa and NPT samples was analysed in order to identify potential PCa biomarkers. The samples were processed according to the manufacturer's protocol using the two-colour Human DNA Methylation 1×244K Microarrays, which interrogate 27,627 known CpG islands (Agilent Technologies, Santa Clara, Calif., USA). Saturated, non-uniform and outlier probe signals were treated as compromised and removed from the analysis. Normalized log ration (Cy5/Cy3) representing methylated/reference DNA was used for further calculations. Probe annotations were uploaded from the SureDesign platform (https://earray.chem.agilent.com/suredesign) and updated using UCSC Genome Browser (https://genome.ucsc.edu) according to the human genome assembly version GRCh38. Probes undetected in ≥30% of all samples were filtered out followed by an additional group comparison-specific filtering leaving only probes detected in 100% of samples in at least 1 of 2 groups to be compared. Fold change (FC) values were estimated and paired or unpaired t-test was applied for group comparisons. Calculations were performed with GeneSpring GX v14.5 software (Agilent Technologies).

The gene set enrichment analysis (GSEA) for the identified differentially methylated genes between groups was performed using publicly available online GSEA tool and Molecular Signatures Database (MSigDB, v5.2; http://software.broadinstitute.org/gsea) [20], both maintained by Broad Institute (Cambridge, Mass., USA). Hallmark genes sets (50 in total) were utilized for GSEA [21]. FDR q-value with the cut-off <0.05 was used for multiple testing correction.

DNA Purification

Up to 60 mg of tissue samples were submerged in liquid nitrogen and mechanically homogenized into powder using cryoPREP™ CP02 Impactor with tissue TUBE TT1 (Covaris, Woburg, Mass., USA). Up to 30 mg of homogenized tissue powder or a total volume of a prepared urine sample were used for the isolation of genomic DNA. Samples were treated with proteinase K (Thermo Scientific™, Thermo Fisher Scientific, Wilmington, Del., USA) in 500 μl of lysis buffer for tissue (50 mM Tris-HCl pH 8.5, 1 mM EDTA, 0.5% Tween-20; all from Carl Roth, Karlsruhe, Germany) or for urine (10 mM Tris-HCl pH 8.0, 1% SDS, 75 mM NaCl; all from Carl Roth) for up to 18 h at 55° C. DNA was extracted following the standard phenol-chloroform purification and ethanol precipitation [18] [22].

The concentration and purity parameters of the extracted DNA were evaluated using the NanoDrop™ 2000 spectrophotometer (Thermo Scientific™). DNA integrity of randomly selected samples was checked electrophoretically using 1.0-1.5% agarose gel.

Bisulfite Conversion

For targeted DNA methylation analysis by means of qualitative or quantitative methylation-specific PCR (MSP or QMSP, respectively), 400 ng of purified DNA were bisulfite-modified using EZ DNA Methylation™ Kit (Zymo Research, Irvine, Calif., USA) according to the manufacturer's protocol, except that the initial incubation step was performed for 15 min at 42° C. The elution was done in 40 μl of elution buffer or PCR-grade water. Modified DNA samples were analysed immediately or stored at ≤−20° C.

The biomarker sequences of fully methylated genetic loci after bisulfite conversion are provided in Table 3.

TABLE 3
The amplicon sequences of the DNA methylation biomarkers.

| Assay | Sequence ID | Sequence type | Sequence (5′→3′) |
|---|---|---|---|
| PRKCB | SEQ ID NO: 1 | MSP amplicon, | TAAGCGTAGTTGGACGAGCGGTAGTAGTTGGGCGAGT |
| | | fully methylated | GATAGTTTCGGTTTCGCGCGTCGCGGTCGTTAGAGTCG |
| | | | GCGTAGGGGAAGCGTTCGCGGTTTCGGGTGTAGTAGC |
| | | | GGTCGTCGTTTT |
| ADAMTS12 | SEQ ID NO: 2 | MSP amplicon, | ACAACGACTACAAAACTACCCGCGATCTCCCTATACTT |
| | | fully methylated | TTTTAAACAAAAAAAAACTAAACACCTTTTTCCCCTCC |
| | | | CTCCTCCTAAAAAAAAAATAATTCAACTAACAATATC |
| | | | CGCTTTCGACGAAATATAAAATAAACCAAAACGAAAA |
| | | | AACGCAACCCACCCCGATCCCCACCCCTCCGCCTCCCG |
| | | | CATACCCCGCGACCTCGCAACCCGCCCGCTCGATACA |
| | | | TCTTCCTCCCGAACTC |
| NAALAD2 | SEQ ID NO: 3 | MSP amplicon, | TATTTATTATGTTCGGGTTATTGCGGGATTTATAGAAT |
| | | fully methylated | GGAAGTTGTTCGTTAATAGGAAGAATGTTTTTTTTTTT |
| | | | TGTAGGGTTTTTTTTTTTTTATCGAGGGTTTTTGGGGAT |
| | | | TATAGGTTTTTAGCGGGTAGGGCGGAGGCGTGGTTTT |
| | | | GCGAAGGTTAGCGGAGGTTATTTAGAGTTTATAGTTTT |
| | | | TTGTTAGCGCGTTTTTTGTTTTTTTGTAGTTTCGAAGTT |
| | | | CGCGAATGTAGTAGG |
| FILIP1L | SEQ ID NO: 4 | MSP amplicon, | CGACCTATAAACGTTACGTCACTATTCTACCTTATAAA |
| | | fully methylated | ACGCTCCGCGTATACGACGCTATCGACGAAAACGCCG |
| | | | ATAACCGCGAAACCCTCGACCGCGACGACGACGCAAC |
| | | | CACACACCCCAACTCCCGCGAATATTCCGACCGTATA |
| | | | AACGAACCGTA |
| ZMIZ1 | SEQ ID NO: 5 | MSP amplicon, | TCGTTTCGAAAATTTTTTAAATCGAGATTTAATTTGGA |
| | | fully methylated | TGTTTAGTTTCGTTTTTTTTTTTTTTTTTTTTTTTTTTTTT |
| | | | CGGTCGAGGTTTTTTTTAGTTTAGTTTTTTTTTTTATTTT |
| | | | TTTTTCGTCGTGGATTTTTATTAGTATGTTTATTTGGGA |
| | | | GGATTCGTTGGGGGGCGGGAGATATTCGAAGTTATTT |
| | | | ATCGTTAGCGTTTTTTCGGCGGTTTTTTCGGGCGATAG |
| | | | CGTTTCGGGAGTT |
| KCTD8 | SEQ ID NO: 6 | MSP amplicon, | CTCCGCGTACTCCTAACGCTCTACGCCCTCGAACTAAA |
| | | fully methylated | CGACGCGTTCCTCCGACCGAAACGACCCCGCTCAAAA |
| | | | TTCGAAACAACGACGACGTCGACGACGCCCGAACTCC |
| | | | ATCGAAAAAAAAACGCGCGAAAAAAAAACTCCGCCG |
| | | | ATACGACGACGACAATAAAAA |
| CCDC181 | SEQ ID NO: 7 | MSP amplicon, | CGAAAACGACAAAAATCTACGCAAACGCATACAATAT |
| | | fully methylated | CCTCAAACCACCGACCCCTCCCGACACCCATCCCGAT |
| | | | ACTTACGAAAAACCAATAAAACTAAAATTTCTAAAAA |
| | | | AACGACTAAAAAATCCACGCATCACTAACGTTATAAA |
| | | | AACTCGCGAAATACCG |
| PRKCB | SEQ ID NO: 8 | QMSP amplicon, | CGTAGTTGGGGTTAGCGGTGTTAAGCGTAGTTGGACG |
| | | fully methylated | AGCGGTAGTAGTTGGGCGAGTGATAGTTTCGGTTTCG |
| | | | CGCGTCGCGGTCGTTAGAGTCGGCGTAGGGGAAGCGT |
| | | | TCGCGGTTTCGGGTGTAGTAGCGGTCGTCGTTTT |
| ADAMTS12 | SEQ ID NO: 9 | QMSP amplicon, | TCAACTAACAATATCCGCTTTCGACGAAATATAAAAT |
| | | fully methylated | AAACCAAAACGAAAAAACGCAACCCACCCCGATCCCC |
| | | | ACCCCTCCGCCTCCCGCATACCCCGCGACCTCGCAACC |
| | | | CGCCCGCTCGATACATCTTCCTCCCG |
| NAALAD2 | SEQ ID NO: 10 | QMSP amplicon, | TGCGAAGGTTAGCGGAGGTTATTTAGAGTTTATAGTTT |
| | | fully methylated | TTTGTTAGCGCGTTTTTTGTTTTTTTGTAGTTTCGAAGT |
| | | | TCGCGAATGTAGTAGGCGTTTTAAGTTCGGTTTTTAAG |
| | | | AAGTTATGGCGGAATTTAGGGGTC |
| CCDC181 | SEQ ID NO: 11 | QMSP amplicon, | TATCCTCAAACCACCGACCCCTCCCGACACCCATCCCG |
| | | fully methylated | ATACTTACGAAAAACCAATAAAACTAAAATTTCTAAA |
| | | | AAAACGACTAAAAAATCCACGCATCACTAACGTTATA |
| | | | AAAACTCGCGAAATACCGC |
| MT1E | SEQ ID NO: 12 | QMSP amplicon, | GGAGGAGGGTGGAAGGTAATTTCGGGGAAATTGGGA |
| | | fully methylated | AAGGCGGTTTGGATTTCGGGAATATCGCGTATTTGCG |
| | | | GGGGTATAGTTTTATTCGAGCGAACGG |
| APC | SEQ ID NO: 13 | QMSP amplicon, | GAACCAAAACGCTCCCCATTCCCGTCGAAAACCCGCC |
| | | fully methylated | GATTAACTAAATATAAACGCACGTAACCGACATATAA |
| RASSF1 | SEQ ID NO: 14 | QMSP amplicon, | CCCGTACTTCGCTAACTTTAAACGCTAACAAACGCGA |
| | | fully methylated | ACCGAACGAAACCACAAAACGAACCCCGACTTCAACG |
| | | | C |
| ACTB | SEQ ID NO: 15 | QMSP amplicon, | AACCAATAAAACCTACTCCTCCCTTAAAAATTACAAA |
| | | endogenous | AACCACAACCTAATAAAAAAAATAACCACCACCCAAC |
| | | control | ACACAATAACAAACACAAATTCACAATCCAAAAAACT |
| | | | TACTAAACCTCCTCCATCACCA |

MSP-methylation-specific PCR, QMSP-quantitative methylation-specific PCR.

Qualitative Methylation-Specific PCR

The bisulfite-modified DNA served as a template for MSP with primers specific for methylated and unmethylated DNA. The MSP primers for the genes PRKCB, ADAMTS12, NAALAD2, FILIP1L, ZMIZ1, KCTD8 and CCDC181, overlapping with the location of the microarray probes of interest, were designed with Methyl Primer Express® Software v1.0 (Applied Biosystems™, Thermo Fisher Scientific, Carlsbad, Calif., USA) and ordered from Metabion (Martinsried, Germany) (Table 4). The MSP reaction mix (25 μl) consisted of 1× Maxima® Hot Start Taq PCR buffer, 2.5 mM MgCl₂, 0.4 mM of each dNTP, 1.25 U Maxima® Hot Start Taq DNA Polymerase (Thermo Scientific™), 1 μM of each primer, and the bisulfite-treated DNA equivalent to 10-20 ng of the starting material. The reaction conditions were optimized prior to the analysis and consisted of 5-10 min at 95° C., 35-38 cycles of 45 s at 95° C., primer annealing for 45 s at 55-62° C. (Table 4) and elongation for 45 s at 72° C., followed by 5-10 min at 72° C. Methylation-positive (MC), methylation-negative (unmethylated, UC), and no-template controls (NTC) were routinely included in all MSP assays for each primer pair. Amplified products were analysed in 3% agarose gels. The amplicon sequences obtainable with the primer pairs for methylated DNA are provided in Table 3.

The bisulfite-modified leukocyte DNA from healthy male donors was used as the UC. CpG methyltransferase-treated (Thermo Scientific™) and bisulfite-modified leukocyte DNA served as the MC. The quality of the UC and MC controls was checked by performing the MSP reaction with the standard samples and analysing amplification products in 3% agarose and/or 7.5% polyacrylamide gels. Only UC and MC controls showing specific amplification with respective primer pairs and no amplification with primers specific for methylated and unmethylated DNA, respectively, were used for the MSP assays.

TABLE 4
Qualitative methylation-specific PCR (MSP) primers, used for the assays, and
amplification conditions.

| | | | | Primer | | |
|---|---|---|---|---|---|---|
| | Primer | | | annealing | Number of | Product |
| Assay | type | Sequence ID | Primer sequence (5′→3′) | t° C. | MSP cycles | size, nt |
| PRKCB | M-F | SEQ ID NO: 16 | TAAGCGTAGTTGGACGAGC | 56 | 36 | 124 |
| PRKCB | M-R | SEQ ID NO: 17 | AAAACGACGACCGCTACTAC | | | |
| PRKCB | U-F | SEQ ID NO: 18 | TGTTAAGTGTAGTTGGATGAGT | 56 | | 127 |
| PRKCB | U-R | SEQ ID NO: 19 | AAAACAACAACCACTACTACACC | | | |
| ADAMTS12 | M-F | SEQ ID NO: 20 | GAGTTCGGGAGGAAGATGTATC | 62 | 35 | 241 |
| ADAMTS12 | M-R | SEQ ID NO: 21 | ACAACGACTACAAAACTACCCG | | | |
| ADAMTS12 | U-F | SEQ ID NO: 22 | GAGTTTGGGAGGAAGATGTATT | 62 | | 243 |
| ADAMTS12 | U-R | SEQ ID NO: 23 | AAACAACAACTACAAAACTACCA | | | |
| NAALAD2 | M-F | SEQ ID NO: 24 | TATTTATTATGTTCGGGTTATTGC | 58 | 35 | 244 |
| NAALAD2 | M-R | SEQ ID NO: 25 | CCTACTACATTCGCGAACTTC | | | |
| NAALAD2 | U-F | SEQ ID NO: 26 | GTTATTTATTATGTTTGGGTTATTGT | 58 | | 246 |
| NAALAD2 | U-R | SEQ ID NO: 27 | CCTACTACATTCACAAACTTCAA | | | |
| FILIP1L | M-F | SEQ ID NO: 28 | TACGGTTCGTTTATACGGTC | 57 | 36 | 160 |
| FILIP1L | M-R | SEQ ID NO: 29 | CGACCTATAAACGTTACGTCA | | | |
| FILIP1L | U-F | SEQ ID NO: 30 | GGAATTATGGTTTGTTTATATGGTT | 57 | | 167 |
| FILIP1L | U-R | SEQ ID NO: 31 | CCCAACCTATAAACATTACATCAC | | | |
| ZMIZ1 | M-F | SEQ ID NO: 32 | TCGTTTCGAAAATTTTTTAAATC | 55 | 38 | 246 |
| ZMIZ1 | M-R | SEQ ID NO: 33 | AACTCCCGAAACGCTATC | | | |
| ZMIZ1 | U-F | SEQ ID NO: 34 | TGTAGTTTGTTTTGAAAATTTTTTAAA | 55 | | 252 |
| ZMIZ1 | U-R | SEQ ID NO: 35 | AACTCCCAAAACACTATCACC | | | |
| KCTD8 | M-F | SEQ ID NO: 36 | TTTTTATTGTCGTCGTCGTATC | 58 | 37 | 169 |
| KCTD8 | M-R | SEQ ID NO: 37 | CTCCGCGTACTCCTAACG | | | |
| KCTD8 | U-F | SEQ ID NO: 38 | GTTTTTTTATTGTTGTTGTTGTATT | 58 | | 175 |
| KCTD8 | U-R | SEQ ID NO: 39 | ACCCTCCACATACTCCTAACA | | | |
| CCDC181 | M-F | SEQ ID NO: 40 | CGGTATTTCGCGAGTTTTTATAAC | 57 | 35 | 164 |
| CCDC181 | M-R | SEQ ID NO: 41 | CGAAAACGACAAAAATCTACG | | | |
| CCDC181 | U-F | SEQ ID NO: 42 | TAGTGGTATTTTGTGAGTTTTTATAAT | 57 | | 168 |
| CCDC181 | U-R | SEQ ID NO: 43 | ACAAAAACAACAAAAATCTACACA | | | |

M/U-primers specific for methylated/unmethylated DNA template after bisulfite modification, F/R-forward/reverse primers.

A run was considered valid if the UC control gave a product only with the primers specific for the unmethylated DNA, the MC control was amplified only with the primers specific for the methylated DNA, and there was no amplification observed in NTC controls. A sample was considered methylated at a particular genetic locus if a product amplified with the primers specific for the methylated DNA was detected and there was no non-specific amplification. A sample was considered unmethylated at a particular genetic locus if a specific product was amplified with the primers specific for the unmethylated DNA and not amplified with the primers specific for the methylated DNA.

Quantitative Methylation-Specific PCR

The bisulfite-modified DNA was used as a template for the quantitative methylation analysis by means of quantitative MSP (QMSP). The QMSP primers and hydrolysis probes, specific for the bisulfite-modified methylated DNA, for the genes PRKCB, ADAMTS12, NAALAD2, CCDC181 and MT1E were designed using the MethPrimer software v1.0 (http://www.urogene.org/methprimer) [23]. The primers and probes were designed to overlap at least partly with the MSP primers (Table 5). The primers for ACTB, which are not overlapping with CpG dinucleotides, were selected from the previous study [24] and were included in each run as a normalizing assay for the DNA input. The amplicon sequences are provided in Table 3. QMSP was performed in separate wells in triplicates for each set of primers. The reaction mix (20 μl) consisted of 1× TaqMan® Universal Master Mix II, no UNG (Applied Biosystems™), 300 nM of each primer, 50 nM of probe, and ˜10 ng of bisulfite-converted DNA. All assays were carried out on the Mx3005P qPCR System (Agilent Technologies) under the following conditions: 95° C. for 10 min followed by 50 cycles of 95° C. for 15 s and 60° C. for 1 min. A run was considered valid when routinely included MCs gave a positive signal and there was no amplification in NTC wells. The threshold used for Cq estimation was determined by applying the background-based threshold algorithm from cycles 5 through 10 and setting the adaptive baseline. The methylation level of a particular gene was estimated as a percentage of the methylated reference DNA, which was calculated using the MC as a 100% methylated reference, where X is the gene of interest (Formula 2):

$$Methylation level , \% = \frac{100 \%}{{2}^{( Cq [ X in sample ] - Cq [ ACTB in sample ] ) - ( Cq [ X in MC ] - Cq [ ACTB in MC ] )}}$$

Formula 2. The formula used for calculating the methylation level of the particular gene of interest (X). The methylation level is expressed in percentage. Cq—cycle of quantification value, MC—methylation-positive DNA standard (control sample).

TABLE 5
Quantitative methylation-specific PCR (QMSP) primers and probes, used for the assays.

Product

| | Primer | | | 5′ | 3′ | size, |
|---|---|---|---|---|---|---|
| Assay ID* | type | Sequence ID | Primer sequence (5′-3′) | modification | modification | nt |
| PRKCB | QM-F | SEQ ID NO: 44 | CGTAGTTGGGGTTAGCGGTG | — | — | 145 |
| PRKCB | QM-R | SEQ ID NO: 45 | AAAACGACGACCGCTACTACA | — | — | |
| PRKCB | QM-P | SEQ ID NO: 46 | TTAGAGTCGGCGTAGGGGAAGCG | JOE | BHQ1 | |
| ADAMTS12 | QM-F | SEQ ID NO: 47 | CGGGAGGAAGATGTATCGAGC | — | — | 138 |
| ADAMTS12 | QM-R | SEQ ID NO: 48 | TCAACTAACAATATCCGCTTTCG | — | — | |
| ADAMTS12 | QM-P | SEQ ID NO: 49 | TTTCGTTTTGGTTTATTTTATATTTCG | CY5 | BHQ3 | |
| NAALAD2 | QM-F | SEQ ID NO: 50 | TGCGAAGGTTAGCGGAGGT | — | — | 139 |
| NAALAD2 | QM-R | SEQ ID NO: 51 | GACCCCTAAATTCCGCCATAA | — | — | |
| NAALAD2 | QM-P | SEQ ID NO: 52 | GAAGTTCGCGAATGTAGTAGGCG | FAM | BHQ1 | |
| CCDC181 | QM-F | SEQ ID NO: 53 | GCGGTATTTCGCGAGTTTTTAT | — | — | 131 |
| CCDC181 | QM-R | SEQ ID NO: 54 | TATCCTCAAACCACCGACC | — | — | |
| CCDC181 | QM-P | SEQ ID NO: 55 | AGTATCGGGATGGGTGTCGGGA | FAM | BHQ1 | |
| MT1E | QM-F | SEQ ID NO: 56 | GGAGGAGGGTGGAAGGTAAT | — | — | 100 |
| MT1E | QM-R | SEQ ID NO: 57 | CCGTTCGCTCGAATAAAACTA | — | — | |
| MT1E | QM-P | SEQ ID NO: 58 | ATTTCGGGAATATCGCGTATTTGC | JOE | BHQ1 | |
| APC | QM-F | SEQ ID NO: 59 | GAACCAAAACGCTCCCCAT | — | — | 74 |
| APC | QM-R | SEQ ID NO: 60 | TTATATGTCGGTTACGTGCGTTTATAT | — | — | |
| APC | QM-P | SEQ ID NO: 61 | CCCGTCGAAAACCCGCCGATTA | Cy5 | BHQ3 | |
| RASSF1 | QM-F | SEQ ID NO: 62 | GCGTTGAAGTCGGGGTTC | — | — | 75 |
| RASSF1 | QM-R | SEQ ID NO: 63 | CCCGTACTTCGCTAACTTTAAACG | FAM | BHQ1/ | |
| RASSF1 | QM-P | SEQ ID NO: 64 | ACAAACGCGAACCGAACGAAACCA | | TAMRA | |
| ACTB | QM-F | SEQ ID NO: 65 | TGGTGATGGAGGAGGTTTAGTAAGT | — | — | 133 |
| ACTB | QM-R | SEQ ID NO: 66 | AACCAATAAAACCTACTCCTCCCTTAA | — | — | |
| ACTB | QM-P | SEQ ID NO: 67 | ACCACCACCCAACACACAATAACAAACACA | FAM | BHQ1/ | |
| | | | | | TAMRA | |

*Oligonucleotide sequences for APC, RASSF1 and ACTB were obtained from previous publications
QM-F/R-forward/reverse primer; QM-P-probe, Cy5-cyanine-5, FAM-fluorescein, JOE-4′,5'-dichloro-2′,7′-dimethoxyfluorescein, BHQ1/3-black hole quencher-1/3.

RNA Extraction and cDNA Synthesis

Total RNA samples were used for target gene expression analysis by quantitative PCR (qPCR). MirVana™ miRNA Isolation Kit (Ambion®, Thermo Fisher Scientific, Foster City, Calif., USA) was used for the RNA extraction following the manufacturer's protocol. Briefly, ˜30 mg of homogenized tissue powder was treated with 500 μL Lysis/Binding Buffer and 50 μL of miRNA Homogenate Additive for 10 min in ice-water bath. The total RNA was extracted with 500 μL of acid-phenol:chloroform and purified using the supplied Filter Cartridges. One hundred μL of preheated (95° C.) Elution Solution was used to recover purified RNA. Only samples having high purity parameters and RNA integrity number (RIN) ≥7, as measured using the 2100 Bioanalyzer (Agilent Technologies), were included in the analysis (Table 1). Samples were stored at −80° C. until further use.

For qPCR, 250 ng of the RNA were reverse transcribed (RT) using High Capacity cDNA Reverse Transcription Kit with RNase Inhibitor according to the recommended protocol (Applied Biosystems™)

Transcriptional Gene Expression Analysis

Expression of the genes PRKCB, ADAMTS12, NAALAD2, ZMIZ1, and endogenous control HPRT1 was evaluated using TaqMan® Gene Expression Assays (Hs00176998_m1, Hs00221792_m1, Hs00229594_m1, Hs01119919_m1, Hs00277476_m1, and Hs02800695_m1, respectively; Applied Biosystems™) in triplicates per gene. The reaction mix (20 μL) consisted of 1× TaqMan® Universal Master Mix II, no UNG (Applied Biosystems™), 0.6 μL of TaqMan® assay, and 2 μL of RT reaction product. Amplification was performed on the Mx3005P qPCR System (Agilent Technologies). Thermal cycling conditions consisted of 95° C. for 10 min, followed by 40 cycles of 95° C. for 15 s and 60° C. for 1 min. Multiple NTCs were included in each RT-qPCR run. Data pre-processing was performed with GenEx v6.0.1 software (MultiD Analyses AB, Göteburg, Sweden) and relative gene expression values in a linear scale were used for calculations.

For cDNA synthesis, 250 ng of RNA was reverse transcribed (RT) using High-Capacity cDNA Reverse Transcription Kit with RNase Inhibitor according to the manufacturer's instructions (Applied Biosystems™)

The Cancer Genome Atlas Dataset of Prostate Cancer

Publicly available data from The Cancer Genome Atlas (TCGA) project, a collaboration between the National Cancer Institute (NCI, USA) and National Human Genome Research Institute (NHGRI, USA), were used to verify the significant findings. Clinical annotation of the samples was obtained from the marker TCGA PRAD publication [26]. Global DNA methylation profiling data using Illumina Infinium HumanMethylation450K (HM450) platform and RNA expression data obtained by RNA-seq were utilized in this study. The level 3 data were obtained from the cBioPortal (http://www.cbiopor tal.org) [27] and methHC (http://methhc.mbc.nctu.edu.tw) [28] data analyses portals. Samples with significant degradation levels, as described in [26], were excluded from the analysis yielding 333 tumours and 19 NPT in total.

Statistical Analysis

Statistical analyses were performed using STATISTICA™ v8.0 (StatSoft, Tulsa, Okla., USA) and MedCalc® v12.7 software (MedCalc Software, Ostend, Belgium). All quantitative variables were tested for normality (Shapiro-Wilk, Kolmogorov-Smirnov and Lilliefors tests) and parametric or nonparametric tests were applied respectively. Student's t-test or Mann-Whitney U test were used to compare quantitative variables between two groups, while 2-sided Fisher's exact test was applied for comparison of categorical variables. For multiple group comparison, Kruskal-Wallis H test was applied. Pearson (R_{P}) and/or Spearman's (R_{S}) rank correlation coefficients were calculated to test the associations between two quantitative variables. Parametric tests were applied for the analysis of TCGA data. Biomarker performance was evaluated by analysing Receiver operating curves (ROC) and calculating the area under the curve (AUC). Biomarkers were also evaluated by calculating various test selectivity parameters: sensitivity, specificity, accuracy, positive predictive value (PPV), negative predictive value (NPV), positive likelihood ratio (LR+), negative likelihood ratio (LR−) and Youden index. For survival analysis, Kaplan-Meier curves were compared with log-rank test. Univariate and/or multivariate Cox proportional hazards modelling was performed and hazard ratios (HR) with 95% confidence intervals (CI) were determined. Differences and associations were considered statistically significant at P<0.0500.

Results

Microarray-Based DNA Methylation Profiling for Biomarker Discovery

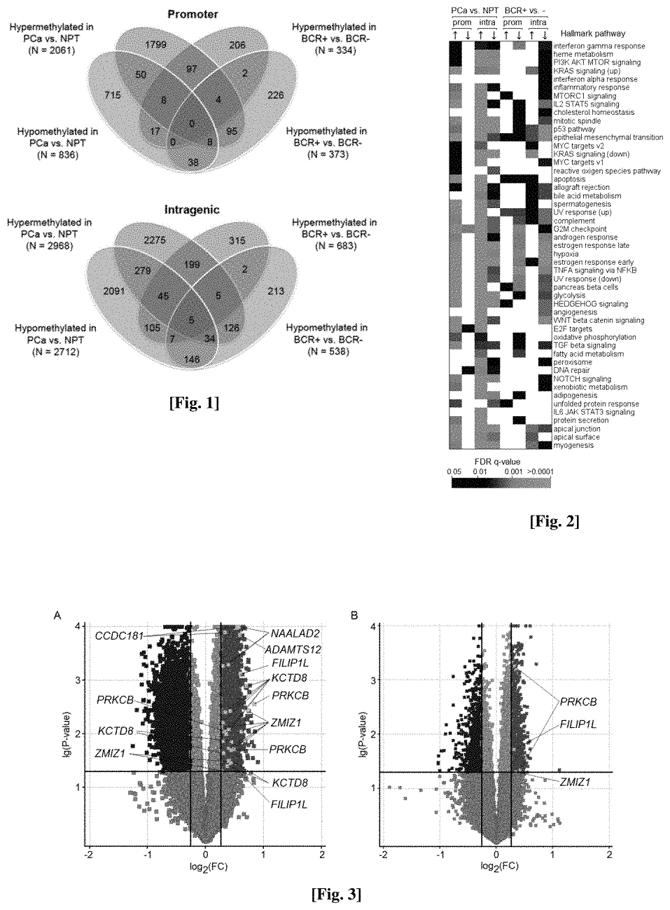

Aiming to identify potential DNA methylation biomarkers of PCa and to elucidate the extent of epigenetic changes in the tumours in general, the genome-wide DNA methylation profile was analysed in 9 pairs of PCa and NPT samples. Significant methylation differences (FC≥1.2, P<0.0500) were associated with 6899 genes in tumours as compared to NPT samples, of which 4227 (61.3%) genes were hypermethylated and 3268 (47.4%) genes were hypomethylated, including 596 (8.6%) genes with concurrent changes observed according to different microarray probes (FIG. 1). The number of hypermethylated genes in promoter region was much higher than the number of hypomethylated genes (72.8% and 29.5%, respectively, with 2.3% overlap), while both events were similarly common in intragenic loci (55.8% and 51.0%, respectively, with 6.8% overlap). Smaller-scale methylation differences were observed comparing BCR-positive and BCR-negative PCa cases (FIG. 1). Of 1804 genes with significant methylation differences, 969 (53.7%) genes were hypermethylated and 868 (48.1%) genes were hypomethylated, including 33 (1.8%) overlapping genes. Increase and decrease of methylation levels were similar in both promoter (53.2% and 47.6%, respectively, with 0.9% overlap) and intragenic loci (44.8% and 56.8%, respectively). Hypermethylation of 411 overlapping genes was detected comparing both PCa vs. NPT and BCR-positive vs. BCR-negative tumour samples, while 291 genes were hypomethylated in both comparisons. Some genes showed both gain and loss of methylation according to different microarray probes.

According to the GSEA analysis, gene sets involved in cell cycle regulation, estrogen response, and apical junction were among the most significantly enriched in PCa as compared to NPT samples (FIG. 2). The increase of methylation levels was the most significant among the genes downregulated in response to ultraviolet (UV) exposure and involved in epithelial-mesenchymal transition (EMT), while decreased methylation was commonly observed in genes associated with mitotic spindle or estrogen response. Similar gene sets (EMT, response to UV) were enriched for hypermethylated genes comparing BCR-positive and BCR-negative cases, while response to androgens or estrogen and hypoxia-related gene sets were enriched for the decrease of methylation (FIG. 2).

Based on methylation differences according to prostate tissue histology and BCR status, as observed in DNA methylome profiling data, and with regard to the GSEA analysis, 7 genes—PRKCB, ADAMTS12, NAALAD2, FILIP1L, ZMIZ1, KCTD8 and CCDC181—were selected as biomarkers for further analysis (FIG. 3).

Qualitative DNA Methylation Analysis at Promoter Loci of the Selected Putative Biomarkers

DNA methylation status of PRKCB, ADAMTS12, NAALAD2, FILIP1L, ZMIZ1 and KCTD8 was analysed qualitatively at regulatory (promoter) regions of the genes. One hundred and fifty-one PCa, 51 NPT, and 17 BPH samples (Table 1) were processed by means of MSP using the primers provided in Table 4. Promoter methylation of the gene CCDC181, previously reported to be frequently methylated in PCa [29], was also included in the analysis. All the samples produced valid results for all of the analysed targets (i.e. 100% of valid samples for each assay).

Methylation of PRKCB, ADAMTS12, NAALAD2, FILIP1L and ZMIZ1, as well as CCDC181 was frequently detected in PCa (up to 90.7%), while less commonly observed in case of KCTD8 (21.2%). Methylation of the genes was significantly more common in tumours as compared to NPT (range 3.9-35.3%) and BPH (0% for all genes; all P<0.0500; FIG. 4). The individual biomarkers, except KCTD8, had moderate to high sensitivity (≤86.1%) and positive predictive values (≤98.2%) for diagnosing early stage/localized PCa, with specificity reaching 100% (when estimated according to the BPH group; Table 6). The biomarkers were also analysed for their diagnostic performance in various combinations. Panels of two or three of the biomarkers showed the best characteristics, which in some cases exceeded the respective values of the individual assays. More specifically, the particular biomarker panels showed increased diagnostic sensitivity and accuracy, and considerably higher NPV (Table 7).

TABLE 6
[The diagnostic test performance characteristics of the analysed methylation
biomarkers in prostate tissues.]

| | | Specificity | | | | | | | Youden |
|---|---|---|---|---|---|---|---|---|---|
| Biomarker | Sensitivity | NPT | BPH | Accuracy | PPV | NPV | LR+ | LR− | index |
| PRKCB | 72.2% | 96.1% | 100% | 78.2% | 98.2% | 53.8% | 18.41 | 0.29 | 0.683 |
| ADAMTS12 | 86.1% | 84.3% | 100% | 85.6% | 94.2% | 67.2% | 5.49 | 0.16 | 0.704 |
| NAALAD2 | 85.4% | 88.2% | 100% | 86.1% | 94.5% | 67.2% | 7.26 | 0.17 | 0.736 |
| FILIP1L | 82.1% | 86.3% | 100% | 83.2% | 94.7% | 62.0% | 5.98 | 0.21 | 0.684 |
| ZMIZ1 | 86.1% | 64.7% | 100% | 80.7% | 87.8% | 61.1% | 2.44 | 0.21 | 0.508 |
| KCTD8 | 21.2% | 98.0% | 100% | 40.6% | 97.0% | 29.6% | 10.81 | 0.80 | 0.192 |
| CCDC181 | 90.7% | 88.2% | 100% | 90.1% | 95.8% | 76.3% | 7.71 | 0.11 | 0.790 |

NPT—noncancerous prostate tissue,
BPH—benign prostatic hyperplasia,
PPV—positive predictive value,
NPV—negative predictive value,
LR+—positive likelihood ratio,
LR−—negative likelihood ratio.
Specificity values determined according to NPT and BPH groups are provided.
Accuracy, PPV, NPV, LR+, LR− and Youden index were calculated using NPT as the control group.

TABLE 7
[The diagnostic test performance characteristics of the selected methylation
biomarker combinations.]

| Biomarker | | Specificity | | | | | | | Youden |
|---|---|---|---|---|---|---|---|---|---|
| combination | Sensitivity | NPT | BPH | Accuracy | PPV | NPV | LR+ | LR− | index |
| PRKCB, | 89.4% | 84.3% | 100% | 88.1% | 94.4% | 72.9% | 5.70 | 0.13 | 0.737 |
| ADAMTS12 | | | | | | | | | |
| PRKCB, | 90.1% | 86.3% | 100% | 89.1% | 95.1% | 74.6% | 6.56 | 0.12 | 0.764 |
| NAALAD2 | | | | | | | | | |
| PRKCB, FILIP1L | 88.1% | 84.3% | 100% | 87.1% | 94.3% | 70.5% | 5.62 | 0.14 | 0.724 |
| PRKCB, | 92.7% | 88.2% | 100% | 91.6% | 95.9% | 80.4% | 7.88 | 0.08 | 0.810 |
| CCDC181 | | | | | | | | | |
| PRKCB, | 93.4% | 76.5% | 100% | 89.1% | 92.2% | 79.6% | 3.97 | 0.09 | 0.699 |
| ADAMTS12, | | | | | | | | | |
| NAALAD2 | | | | | | | | | |
| PRKCB, | 94.0% | 78.4% | 100% | 90.1% | 92.8% | 81.6% | 4.36 | 0.08 | 0.725 |
| ADAMTS12, | | | | | | | | | |
| FILIP1L | | | | | | | | | |
| PRKCB, | 95.4% | 80.4% | 100% | 91.6% | 93.5% | 85.4% | 4.86 | 0.06 | 0.758 |
| ADAMTS12, | | | | | | | | | |
| CCDC181 | | | | | | | | | |
| PRKCB, | 94.0% | 76.5% | 100% | 89.6% | 92.2% | 81.3% | 4.00 | 0.08 | 0.705 |
| NAALAD2, | | | | | | | | | |
| FILIP1L | | | | | | | | | |
| PRKCB, | 95.4% | 62.7% | 100% | 87.1% | 88.3% | 85.1% | 2.56 | 0.07 | 0.581 |
| NAALAD2, ZMIZ1 | | | | | | | | | |
| PRKCB, | 95.4% | 82.4% | 100% | 92.1% | 94.1% | 85.7% | 5.40 | 0.07 | 0.778 |
| NAALAD2, | | | | | | | | | |
| CCDC181 | | | | | | | | | |
| FILIP1L, ZMIZ1 | 93.4% | 62.7% | 100% | 85.6% | 88.1% | 76.2% | 2.51 | 0.11 | 0.561 |
| FILIP1L, | 91.4% | 80.4% | 100% | 88.6% | 93.2% | 75.9% | 4.66 | 0.11 | 0.718 |
| CCDC181 | | | | | | | | | |
| ZMIZ1, CCDC181 | 94.0% | 64.7% | 100% | 86.6% | 88.8% | 78.6% | 2.66 | 0.09 | 0.587 |
| ADAMTS12, | 94.0% | 80.4% | 100% | 90.6% | 93.4% | 82.0% | 4.80 | 0.07 | 0.744 |
| CCDC181 | | | | | | | | | |

NPT—noncancerous prostate tissue,
BPH—benign prostatic hyperplasia,
PPV—positive predictive value,
NPV—negative predictive value,
LR+—positive likelihood ratio,
LR−—negative likelihood ratio.
Specificity values determined according to NPT and BPH groups are provided.
Accuracy, PPV, NPV, LR+, LR− and Youden index were calculated using NPT as the control group.

In the group of men suspected of having PCa and considered for biopsy, the presence of the biomarker methylation was associated with up to 95.2% probability of having PCa, when tested in prostate tissue, indicating the utility of such assays for more accurate diagnostics in a high-risk population (Table 8). Moreover, as estimated according to the PCa prevalence rates in 2018, obtained from the International Agency for Research on Cancer (https://gco.iarc.fr/today/), the presence of methylation as measured by the individual biomarkers or their combinations was associated with the increase in the probability of having PCa from 2.4 to 17.8 times, further supporting the potential value of the biomarkers for PCa detection (Table 9).

TABLE 8
[The post-test probability estimates for diagnosing PCa in an individual
when analysing the particular biomarkers or their selected combinations
in men suspected of having PCa and undergoing prostate biopsy.]

| | When pre-test | | When pre-test | |
|---|---|---|---|---|
| | probability | | probability is | |
| | is 45.0%* | | 51.8%** | |
| | Post-test | Absolute | Post-test | Absolute |
| Biomarker assay(-s) | probability | difference | probability | difference |
| PRKCB | 93.8% | 48.8% | 95.2% | 43.4% |
| ADAMTS12 | 81.8% | 36.8% | 85.5% | 33.7% |
| NAALAD2 | 85.6% | 40.6% | 88.6% | 36.8% |
| FILIP1L | 83.0% | 38.1% | 86.5% | 34.7% |
| ZM/Z1 | 66.6% | 21.6% | 72.4% | 20.6% |
| KCTD8 | 89.8% | 44.9% | 92.1% | 40.3% |
| CCDC181 | 86.3% | 41.3% | 89.2% | 37.4% |
| PRKCB, ADAMTS12 | 82.3% | 37.4% | 86.0% | 34.2% |
| PRKCB, NAALAD2 | 84.3% | 39.3% | 87.6% | 35.8% |
| PRKCB, FILIP1L | 82.1% | 37.1% | 85.8% | 34.0% |
| PRKCB, CCDC181 | 86.6% | 41.6% | 89.4% | 37.6% |
| ADAMTS12, CCDC181 | 79.7% | 34.7% | 83.8% | 31.9% |
| PRKCB, ADAMTS12, | 76.4% | 31.5% | 81.0% | 29.2% |
| NAALAD2 | | | | |
| PRKCB, ADAMTS12, | 78.1% | 33.1% | 82.4% | 30.6% |
| FILIP1L | | | | |
| PRKCB, ADAMTS12, | 79.9% | 34.9% | 83.9% | 32.1% |
| CCDC181 | | | | |
| PRKCB, NAALAD2, | 76.6% | 31.6% | 81.1% | 29.3% |
| FILIP1L | | | | |
| PRKCB, NAALAD2, | 67.7% | 22.7% | 73.3% | 21.5% |
| ZMIZ1 | | | | |
| PRKCB, NAALAD2, | 81.5% | 36.6% | 85.3% | 33.5% |
| CCDC181 | | | | |
| FILIP1L, ZMIZ1 | 67.2% | 22.2% | 72.9% | 21.1% |
| FILIP1L, CCDC181 | 79.2% | 34.2% | 83.4% | 31.6% |
| ZMIZ1, CCDC181 | 68.5% | 23.6% | 74.1% | 22.3% |

*According to the 2017 data by the National Health Insurance Fund under the Ministry of Health of Lithuania.
**According to study by Riedinger et al. for the Michigan Urological Surgery Improvement Collaborative (2014) [30]

TABLE 9
[Increase of the probability (as a fold change) of correctly
diagnosing PCa in an individual when analysing the particular
biomarkers with regard to the varying PCa prevalence rates in
specific regions.]

| | | North | |
|---|---|---|---|
| Biomarker assay(-s) | Europe | America | World |
| PRKCB | 17.1 | 17.1 | 17.8 |
| ADAMTS12 | 5.4 | 5.4 | 5.4 |
| NAALAD2 | 7.1 | 7.1 | 7.2 |
| FILIP1L | 5.9 | 5.9 | 5.9 |
| ZM/Z1 | 2.4 | 2.4 | 2.4 |
| KCTD8 | 10.4 | 10.3 | 10.6 |
| CCDC181 | 7.5 | 7.5 | 7.6 |
| PRKCB, ADAMTS12 | 5.6 | 5.6 | 5.7 |
| PRKCB, NAALAD2 | 6.4 | 6.4 | 6.5 |
| PRKCB, FILIP1L | 5.5 | 5.5 | 5.6 |
| PRKCB, CCDC181 | 7.7 | 7.6 | 7.8 |
| PRKCB, ADAMTS12, NAALAD2 | 3.9 | 3.9 | 3.9 |
| PRKCB, ADAMTS12, FILIP1L | 4.3 | 4.3 | 4.3 |
| PRKCB, ADAMTS12, CCDC181 | 4.8 | 4.8 | 4.8 |
| PRKCB, NAALAD2, FILIP1L | 3.9 | 3.9 | 4.0 |
| PRKCB, NAALAD2, ZMIZ1 | 2.5 | 2.5 | 2.6 |
| PRKCB, NAALAD2, CCDC181 | 5.3 | 5.3 | 5.4 |
| FILIP1L, ZMIZ1 | 2.5 | 2.5 | 2.5 |
| FILIP1L, CCDC181 | 4.6 | 4.6 | 4.6 |
| ZMIZ1, CCDC181 | 2.6 | 2.6 | 2.7 |
| ADAMTS12, CCDC181 | 4.7 | 4.7 | 4.8 |

The 2018 data of prostate cancer prevalence rates were obtained from the International Agency for Research on Cancer (https://gco.iarc.fr/today/).

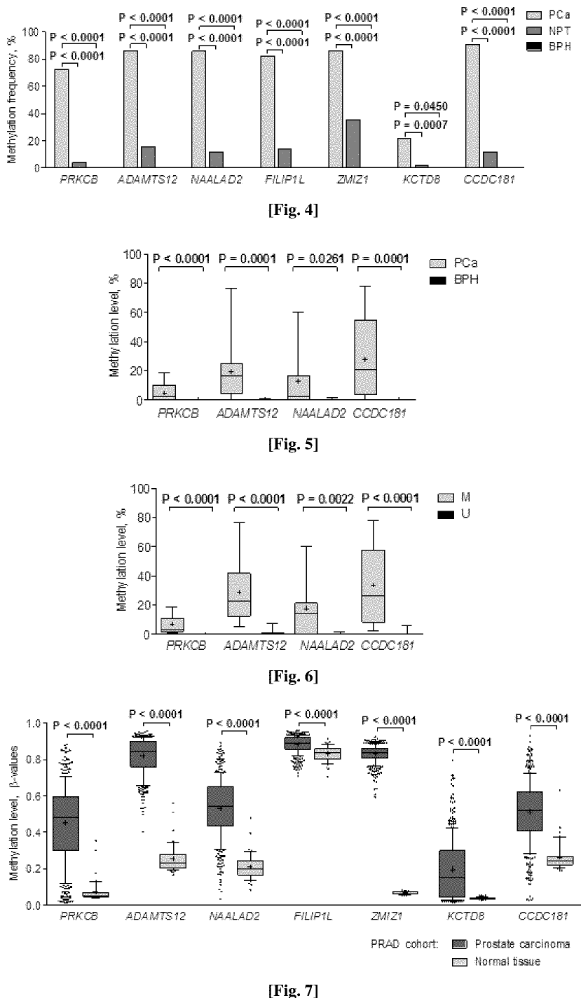

Based on the above-mentioned values of the test performance, the genes PRKCB, ADAMTS12, NAALAD2 and CCDC181 were selected for the quantitative methylation analysis by means of QMSP using the designed primer and probe sequences provided in Table 5. The analysis showed that methylation levels of the genes were significantly higher in randomly selected 15 PCa samples than in 15 BPH samples (all P<0.0500; FIG. 5). The methylation levels also significantly corresponded to the qualitative results obtained by means of MSP (all P<0.0500; FIG. 6).

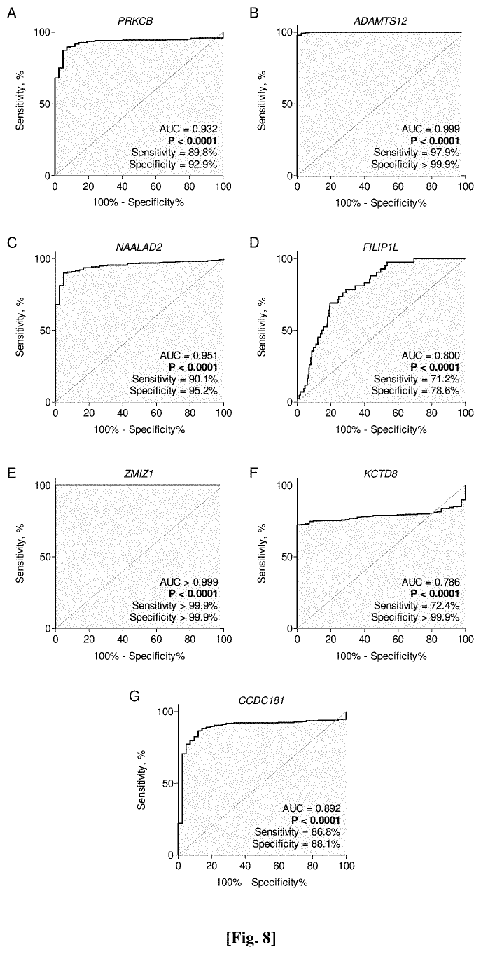

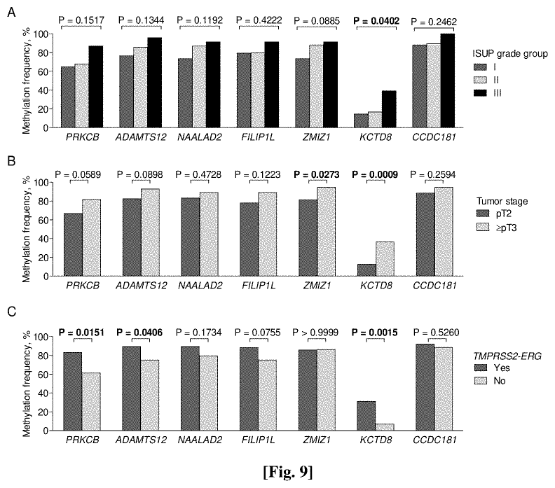

To confirm our findings, the PRAD dataset of TCGA was used (333 cases in total) [26]. In accordance with our data, significantly higher methylation levels were identified in tumours as compared to normal tissues for all the genes (all P<0.0001; FIG. 7). Additionally, in tumours, methylation levels of KCTD8 (median β-value 0.15) were lower than those of the other genes (median β-values ≥0.48), while FILIP1L was characterized by relatively high methylation in normal tissues (median β-values 0.89 and 0.84 in tumours and normal tissues, respectively). ROC curve analysis revealed high diagnostic sensitivity and specificity for PCa according to the methylation levels of PRKCB, ADAMTS12, NAALAD2, ZMIZ1 and CCDC181, while FILIP1L and KCTD8 had somewhat lower values (FIG. 8).

Aberrant promoter methylation of the genes was further analysed according to clinical-pathological patients' characteristics in the test cohort (FIG. 9 and Table 10). Methylation frequencies of most of the genes showed an increasing tendency according to the higher ISUP grade group; however, the observed association was statistically significant only for KCTD8 (P=0.0402; FIG. 9A). ZMIZ1 and KCTD8 were more frequently methylated in ≥pT3 tumours as compared to pT2 (P=0.0273 and P=0.0009, respectively; FIG. 9B). Furthermore, PRKCB, ADAMTS12 and KCTD8 were more commonly methylated in tumours expressing TMPRSS2-ERG fusion transcript (all P<0.0500; FIG. 9C). No associations between promoter methylation and PSA level, prostate mass, or patients' age were detected.

TABLE 10
[Associations of promoter methylation and gene expression with clinical-pathological variables and the TMPRSS2-ERG fusion status
in prostate tumours.]

| Promoter | ≥pT3 vs. pT2 | | ISUP grade group | | TMPRSS2-ERG+ vs. − | | PSA | | Prostate mass | | Age | |
|---|---|---|---|---|---|---|---|---|---|---|---|---|
| methylation | Frequency, % | P-value | H | P-value | Frequency, % | P-value | Z_{ad} | P-value | Z_{ad} | P-value | Z_{ad} | P-value |
| PRKCB | 81.8 vs. 66.7 | 0.0589 | 7.19 | 0.0659 | 83.1 vs. 61.4 | 0.0151 | 1.91 | 0.0555 | −0.17 | 0.4433 | 0.11 | 0.9123 |
| ADAMTS12 | 92.7 vs. 82.3 | 0.0898 | 5.58 | 0.1338 | 89.6 vs. 75.0 | 0.0406 | 0.43 | 0.6665 | −0.70 | 0.4859 | 1.28 | 0.2021 |
| NAALAD2 | 89.1 vs. 83.3 | 0.4728 | 5.91 | 0.2062 | 89.6 vs. 79.6 | 0.1734 | 1.86 | 0.0629 | 0.34 | 0.7357 | −0.74 | 0.4599 |
| FILIP1L | 89.1 vs. 78.1 | 0.1223 | 1.93 | 0.5875 | 88.3 vs. 75.0 | 0.0755 | 0.70 | 0.4810 | 0.70 | 0.4810 | −0.12 | 0.9071 |
| ZMIZ1 | 94.5 vs. 81.3 | 0.0273 | 6.46 | 0.0914 | 85.7 vs. 86.4 | >0.9999 | 1.94 | 0.0528 | 0.58 | 0.5593 | 1.41 | 0.1577 |
| KCTD8 | 36.4 vs. 12.5 | 0.0008 | 6.49 | 0.0902 | 31.2 vs. 6.8 | 0.0015 | 0.98 | 0.3248 | −0.49 | 0.6275 | 0.44 | 0.6568 |
| CCDC181 | 94.5 vs. 88.5 | 0.2594 | 2.85 | 0.4150 | 92.2 vs. 88.6 | 0.5260 | 0.22 | 0.8275 | 1.22 | 0.2237 | 1.30 | 0.1924 |
| Gene | pT | | ISUP grade group | | TMPRSS2-ERG+ vs. − | | PSA | | Prostate mass | | Age | |
| expression | Z_{ad} | P-value | R_{S} | P-value | Z_{ad} | P-value | R_{S} | P-value | R_{S} | P-value | R_{S} | P-value |
| PRKCB | −0.45 | 0.6528 | −0.16 | 0.1439 | 0.17 | 0.8674 | −0.17 | 0.1362 | 0.00 | 0.9815 | 0.13 | 0.2456 |
| ADAMTS12 | −0.35 | 0.7248 | 0.13 | 0.2570 | 0.97 | 0.3323 | −0.12 | 0.2958 | 0.05 | 0.6676 | 0.01 | 0.9334 |
| NAALAD2 | −1.07 | 0.2864 | −0.35 | 0.0015 | −0.03 | 0.9742 | −0.19 | 0.0972 | −0.03 | 0.8003 | 0.27 | 0.0153 |
| ZMIZ1 | −0.29 | 0.7692 | −0.07 | 0.5211 | −0.09 | 0.9313 | 0.11 | 0.3433 | 0.03 | 0.8052 | 0.02 | 0.8385 |
| CCDC181 | −0.65 | 0.5154 | −0.35 | 0.0016 | 2.47 | 0.0136 | −0.20 | 0.0755 | −0.01 | 0.9482 | 0.15 | 0.1698 |

pT—pathological tumour stage,
ISUP—International Society of Urological Pathology,
TMPRSS2-ERG +/− TMPRSS2-ERG fusion positive/negative status,
PSA—prostate-specific antigen,
H—Kruskal-Wallis's H parameter,
Z_{ad}—Mann-Whitney's Z adjusted parameter,
R_{S}—Spearman's correlation coefficient.
Significant P-values are in bold.

Transcriptional Expression Analysis of the Selected Target Genes

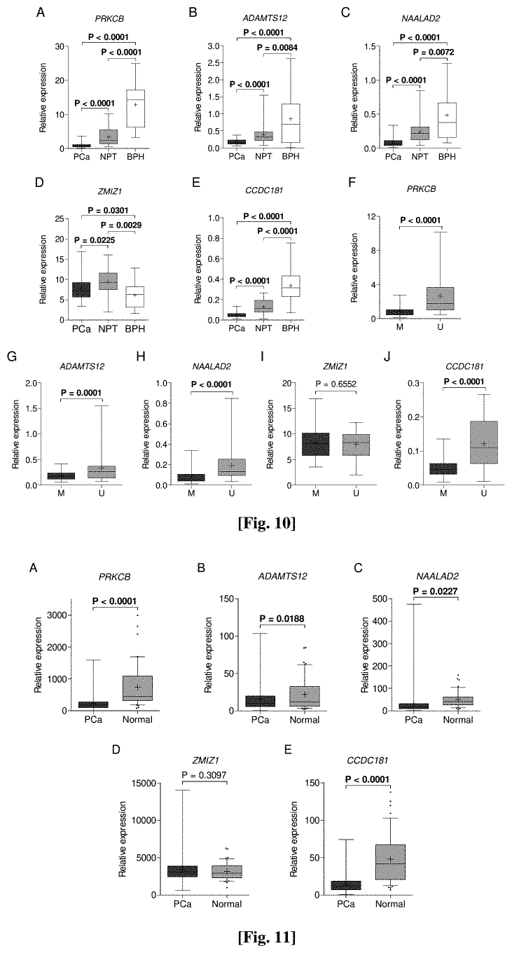

Based on the methylation frequencies and with regard to the associations with clinical-pathological variables and the fusion transcript status, the genes PRKCB, ADAMTS12, NAALAD2, ZMIZ1 and CCDC181 were further submitted to the expression analysis at the transcriptional level. RNA of sufficient quality was available of 81 PCa, 25 NPT and 17 BPH samples (Table 1). Expression levels of PRKCB, ADAMTS12, NAALAD2 and CCDC181 were significantly lower in PCa as compared to NPT and BPH samples (all P<0.0500). In the case of ZMIZ1, lower expression was observed in PCa than in NPT, but higher than in BPH samples (all P<0.0500; FIG. 10A-E). Furthermore, lower expression levels of the analysed genes, except ZMIZ1, in tissues of PCa patients correlated with methylated promoter status (P≤0.0001 for PRKCB, ADAMTS12, NAALAD2 and CCDC181), proving DNA methylation as a regulatory mechanism responsible for the altered gene expression (FIG. 10F-J).

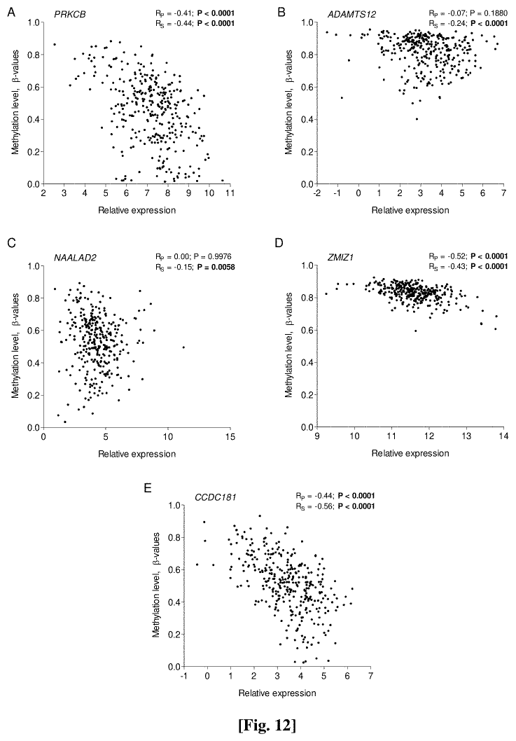

Consistently, lower expression levels of PRKCB, ADAMTS12, NAALAD2 and CCDC181 were observed in the tumours as compared to the normal tissues in the PRAD cohort of TCGA (all P<0.0500; FIG. 11). PRKCB, ZMIZ1 and CCDC181 were expressed at lower levels in the PCa samples with higher methylation levels (all P<0.0500), while weaker associations were observed for ADAMTS12 and NAALAD2 (P>0.0500; FIG. 12).

In the test cohort, decreasing expression levels of NAALAD2 and CCDC181 correlated with the higher ISUP grade group (P=0.0015 and P=0.0016, respectively; Table 10). Higher expression levels of CCDC181 were specific for tumours expressing the TMPRSS2-ERG transcript (P=0.0136). No associations between gene expression and tumour stage pT, PSA level or prostate mass were identified. However, in the test cohort, the expression of NAALAD2 positively correlated with patients' age (R_{S}=0.27, P=0.0153; Table 10), but this association was not supported by the TCGA data (P>0.0500; not shown).

Biochemical Recurrence-Free Survival Analysis

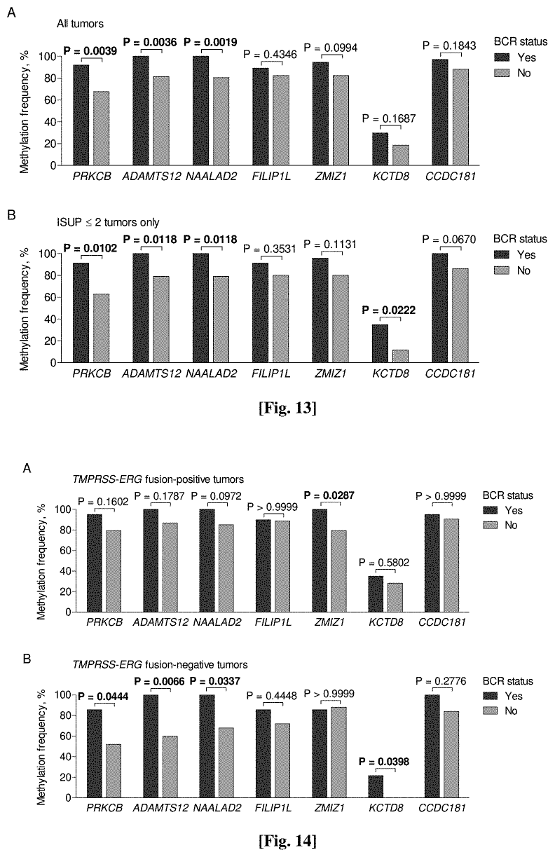

To investigate the performance of the genes for predicting progression in localized or locally advanced PCa cases, the BCR-free survival analysis was performed. Aberrant methylation of PRKCB, ADAMTS12 and NAALAD2 was more frequent in BCR-positive than in BCR-negative cases (P=0.0039, P=0.0036 and P=0.0019, respectively; FIG. 13A). More specifically, these genes, together with KCTD8, were more frequently methylated in a subgroup of ISUP grade group 1 or 2 tumours (all P<0.0500; FIG. 13B). Moreover, higher methylation frequencies of the four genes were also significantly associated with BCR in a subgroup of TMPRSS2-ERG fusion-negative tumours, while the methylation status of ZMIZ1 showed a similar association in the fusion-positive cases (all P<0.0500; FIG. 14).

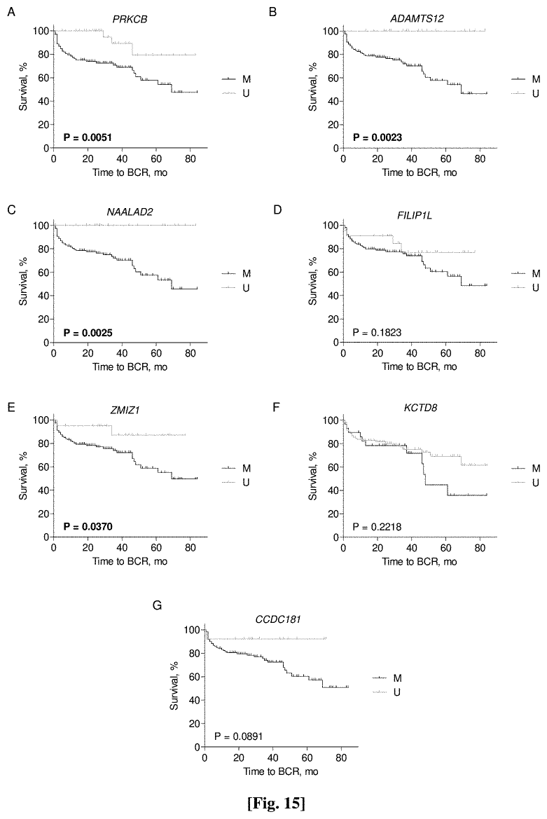

The prognostic potential of the genes was further analysed by comparing Kaplan-Meier curves. The analysis showed significantly lower BCR-free survival rate in PCa cases with methylated status of PRKCB, ADAMTS12, NAALAD2 and ZMIZ1 (all P<0.0500), while no associations were observed for FILIP1L, KCTD8 or CCDC181 (all P>0.0500; FIG. 15). The significance of PRKCB, ADAMTS12, NAALAD2 and ZMIZ1 methylation as an independent prognostic factor was also supported by univariate and multivariate Cox proportional hazard analyses in the test cohort (Tables 11 and 12). Various models of two or more methylation biomarkers significantly predicted BCR-free survival, with PRKCB, ADAMTS12 and NAALAD2 showing the best performance. Altogether, this indicates the potential to develop a molecular test for predicting PCa progression based solely on DNA methylation biomarkers. The models including methylation biomarkers together with the evaluation of TMPRSS2-ERG transcript status also showed prognostic value. Furthermore, inclusion of patient's age and/or PSA level could provide improved prognostic power. Selected multivariate models are provided in Table 12.

TABLE 11
[Univariate Cox proportional hazard analysis of the gene methylation
biomarkers in prostate tissues and other variables.]

| Biomarker | Lithuanian cohort | | TCGA cohort | |
|---|---|---|---|---|
| assay(-s) | HR [95% CI] | P-value | HR [95% CI] | P-value |

Promoter methylation

| | | | | |
|---|---|---|---|---|
| PRKCB | 4.4 [1.4; 14.3] | 0.0025 | 4.8 [0.8; 29.2] | 0.0795 |
| ADAMTS12 | >1000 | 0.0002 | 51.2 [0.6; >1000] | 0.0624 |
| NAALAD2 | >1000 | 0.0002 | 7.3 [0.7; 77.0] | 0.0911 |
| FILIP1L | 1.9 [0.7; 5.4] | 0.1827 | 722.4 [0.1; >1000] | 0.1497 |
| ZM/Z1 | 3.7 [0.9; 15.3] | 0.0278 | 0.2 [0; 89.1] | 0.6455 |
| KCTD8 | 1.5 [0.8; 3.1] | 0.2416 | 1.1 [0.1; 8.5] | 0.9625 |
| CCDC181 | 4.4 [0.6; 31.7] | 0.0611 | 7.0 [0.8; 59.2] | 0.0702 |

Clinical-pathological variables

| | | | | |
|---|---|---|---|---|
| pT (>3 vs. 2) | 4.68 [2.30; 9.51] | <0.0001 | 8.6 [2.1; 35.9] | 0.0001 |
| ISUP grade | 2.93 [2.06; 4.16] | <0.0001 | 2.0 [1.5; 2.8] | <0.0001 |
| group | | | | |
| (1 to 5) | | | | |
| PSA (cont.) | 1.02 [1.00; 1.04] | 0.0342 | 1.0 [1.0; 1.1] | 0.4881 |
| Age (cont.) | 1.00 [0.95; 1.04] | 0.9241 | 1.0 [1.0; 1.1] | 0.4655 |

Gene expression

| | | | | |
|---|---|---|---|---|
| TMPRSS2- | 0.70 [0.34; 1.44] | 0.3328 | n.a. | n.a. |
| ERG | | | | |
| (yes vs. no) | | | | |

For calculations, methylation status was used in the test cohort, while methylation level—in TCGA.
pT—pathological tumour stage,
ISUP—International Society of Urological Pathology,
PSA—prostate-specific antigen,
HR—hazard ratio,
CI—confidence intervals,
n.a.—not available.
Significant P-values are in bold.

TABLE 12
[Multivariate Cox proportional hazard analysis of the gene methylation biomarkers in prostate
tumours and other variables.]

| | Test cohort | | TCGA cohort | |
|---|---|---|---|---|
| Biomarker assay(-s) | HR (95% CI) | P-value | HR (95% CI) | P-value |

Combinations of gene methylation assays

| | | | | |
|---|---|---|---|---|
| PRKCB, ADAMTS12 | >1000 | 0.0001 | >1000 | 0.0511 |
| PRKCB, NAALAD2 | >1000 | <0.0001 | 687.6 [0.8; >1000] | 0.0674 |
| PRKCB, FILIP1L | 274.2 [3.1; >1000] | 0.0034 | 586.8 [0.4; >1000] | 0.0925 |
| PRKCB, CCDC181 | 329.0 [3.7; >1000] | <0.0001 | 646.4 [1.4; >1000] | 0.0479 |
| PRKCB, ADAMTS12, NAALAD2 | >1000 | 0.0122 | >1000 | 0.0402 |
| PRKCB, ADAMTS12, FILIP1L | 428.0 [9.2; >1000] | 0.0002 | >1000 | 0.0504 |
| PRKCB, ADAMTS12, CCDC181 | >1000 | 0.0001 | 922.6 [1.4; >1000] | 0.0440 |
| PRKCB, NAALAD2, FILIP1L | 387.4 [12.9; >1000] | 0.0001 | 712.1 [0.9; >1000] | 0.0628 |
| PRKCB, NAALAD2, ZMIZ1 | >1000 | 0.0001 | 48.6 [0.6; >1000] | 0.1124 |
| PRKCB, NAALAD2, CCDC181 | >1000 | <0.0001 | >1000 | 0.0312 |
| FILIP1L, ZMIZ1 | >1000 | 0.0187 | 153.9 [0.1; >1000] | 0.2124 |
| FILIP1L, CCDC181 | 658.5 [0.1; >1000] | 0.0717 | 458.1 [0.8; >1000] | 0.0693 |
| ZMIZ1, CCDC181 | 718.8 [1.1; >1000] | 0.0158 | 143.0 [0.9; >1000] | 0.0693 |
| ADAMTS12, CCDC181 | >1000 | 0.0003 | 856.8 [1.0; >1000] | 0.0537 |
| ADAMTS12, NAALAD2 | >1000 | <0.0001 | >1000 | 0.0345 |

Combinations with the fusion transcript status

| | | | | |
|---|---|---|---|---|
| PRKCB, TMPRSS2-ERG | 47.7 [2.5; 909.9] | 0.0079 | n.a. | n.a. |
| ADAMTS12, TMPRSS2-ERG | 214.5 [9.9; >1000] | 0.0001 | n.a. | n.a. |
| NAALAD2, TMPRSS2-ERG | 351.5 [9.0; >1000] | 0.0003 | n.a. | n.a. |
| PRKCB, ADAMTS12, TMPRSS2- | 96.8 [8.6; >1000] | 0.0001 | n.a. | n.a. |
| ERG | | | | |
| PRKCB, NAALAD2, TMPRSS2- | 101.9 [8.0; >1000] | 0.0001 | n.a. | n.a. |
| ERG | | | | |
| ADAMTS12, NAALAD2, | 272.1 [16.5; >1000] | <0.0001 | n.a. | n.a. |
| TMPRSS2-ERG | | | | |
| PRKCB, ADAMTS12, | 143.2 [14.1; >1000] | <0.0001 | n.a. | n.a. |
| NAALAD2, TMPRSS2-ERG | | | | |

Combinations with PSA and age

| | | | | |
|---|---|---|---|---|
| PRKCB, PSA | 105.3 [7.8; >1000] | 0.0007 | >1000 | 0.3762 |
| ADAMTS12, PSA | 154.1 [10.1; >1000] | 0.0005 | >1000 | 0.0049 |
| NAALAD2, PSA | 59.6 [8.3; 427.9] | 0.0002 | 234.9 [0.0; >1000] | 0.6028 |
| CCDC181, PSA | 42.6 [2.5; 739.0] | 0.0212 | >1000 | 0.0245 |
| PRKCB, PSA, age | 105.5 [7.8; >1000] | 0.0007 | >1000 | 0.1408 |
| ADAMTS12, PSA, age | 152.7 [10.2; >1000] | 0.0004 | >1000 | 0.0063 |
| NAALAD2, PSA, age | 61.1 [8.3; 451.0] | 0.0003 | >1000 | 0.1817 |
| PRKCB, NAALAD2, PSA, age | 85.6 [11.5; 640.3] | <0.0001 | >1000 | 0.1543 |
| Miscellaneous | | | | |
| PRKCB, TMPRSS2-ERG, age | 49.8 [2.7; 933.4] | 0.0070 | n.a. | n.a. |
| ADAMTS12, TMPRSS2-ERG, | 197.8 [10.4; >1000] | 0.0001 | n.a. | n.a. |
| age | | | | |
| NAALAD2, TMPRSS2-ERG, age | 355.9 [9.1; >1000] | 0.0003 | n.a. | n.a. |
| PRKCB, TMPRSS2-ERG, PSA, | 46.9 [3.2; 689.8] | 0.0058 | n.a. | n.a. |
| age | | | | |
| ADAMTS12, TMPRSS2-ERG, | | | | |
| PSA, age | 186.9 [9.8; >1000] | 0.0002 | n.a. | n.a. |
| NAALAD2, TMPRSS2-ERG, | 61.3 [7.0; 536.1] | 0.0004 | n.a. | n.a. |
| PSA, age | | | | |
| PRKCB, NAALAD2, TMPRSS2- | 56.6 [7.4; 434.5] | 0.0001 | n.a. | n.a. |
| ERG, PSA | | | | |

For calculations, methylation status was used in the test cohort, while methylation level—in TCGA.
Age and prostate-specific antigen (PSA) level were treated as continuous variables, whereas TMPRSS2-ERG fusion transcript status—as an alternative variable (yes/no).
HR—hazard ratio,
CI—confidence intervals,
n.a.—not available.
Significant P-values are in bold.

In TCGA data analysis, PCa cases with prior cancer diagnosis and/or prior neoadjuvant therapy, and metastatic cases were filtered out in order to better match the test cohort. Methylation levels of the analysed genes were not associated with BCR status in univariate Cox models. However, close to significant associations were observed for PRKCB, ADAMTS12, NAALAD2, CCDC181 and for several biomarker combinations. This was most likely due to a smaller proportion of BCR-positive cases as compared to the test cohort (11.8% vs. 27.2%, respectively) and the dominance of advanced (i.e. ISUP 4 or 5) PCa cases (38.8% vs. 5.8%, respectively).

DNA Methylation Analysis in Urine

In urine samples, DNA methylation analysis was performed by the QMSP method, after the optimization of the reaction conditions, and was evaluated both quantitatively and qualitatively. For the quantitative evaluation of the particular biomarker, the methylation levels determined according to Formula 2 were used. The qualitative interpretation of the results was made by applying three alternative thresholds to the obtained methylation levels: a) the gene-specific average methylation level in the ASC group (the ASC-based threshold), b) the gene-specific average methylation level in the BPH group (the BPH-based threshold), and c) methylation level above 0.1% (the 0.1 C threshold), as calculated according to Formula 2. The particular gene was considered as having methylation status when its methylation level was above the particular threshold (Table 13).

TABLE 13
[The thresholds applied for the qualitative interpretation of the gene
methylation levels.]

| | Threshold value, % | | | |
|---|---|---|---|---|
| Parameter | PRKCB | ADAMTS12 | NAALAD2 | CCDC181 |
| ASC-based, M | >0.015 | >0.248 | >0.071 | >0.437 |
| ASC-based, U | ≤0.015 | ≤0.248 | ≤0.071 | ≤0.437 |
| BPH-based, M | >0.011 | >0.043 | >0.076 | >0.003 |
| BPH-based, U | ≤0.011 | ≤0.043 | ≤0.076 | ≤0.003 |
| 0.1C threshold, M | >0.100 | >0.100 | >0.100 | >0.100 |
| 0.1C threshold, U | ≤0.100 | ≤0.100 | ≤0.100 | ≤0.100 |

ASC—asymptomatic controls;
BPH—benign prostatic hyperplasia;
M/U—methylated/unmethylated status.

For the quantitative evaluation of a combination of biomarkers, an empirical algorithm has been developed which provides a derivative estimate xMI based on the methylation levels, as obtained using Formula 2 and expressed in percentage, of a number of biomarkers included in the panel (at least 2 biomarkers; Formula 3).

xMI=1000N×Σᵢ₌₁^{N}Xi/(1+2000Σ_{Xi<0.1}^{N}1)

Formula 3. The algorithm used for calculating the derivative estimate of methylation xMI for a combination of biomarkers. X—the methylation level of a particular gene, N—the number of genes included in a panel (N≥2).

The derivative estimates xMI of two of the possible biomarker combinations were further analysed in detail, namely the combination of PRKCB, ADAMTS12 and NAALAD2, hereinafter referred to as xMI3, and the combination of PRKCB, ADAMTS12, NAALAD2 and CCDC181, hereinafter referred to as xMI4.

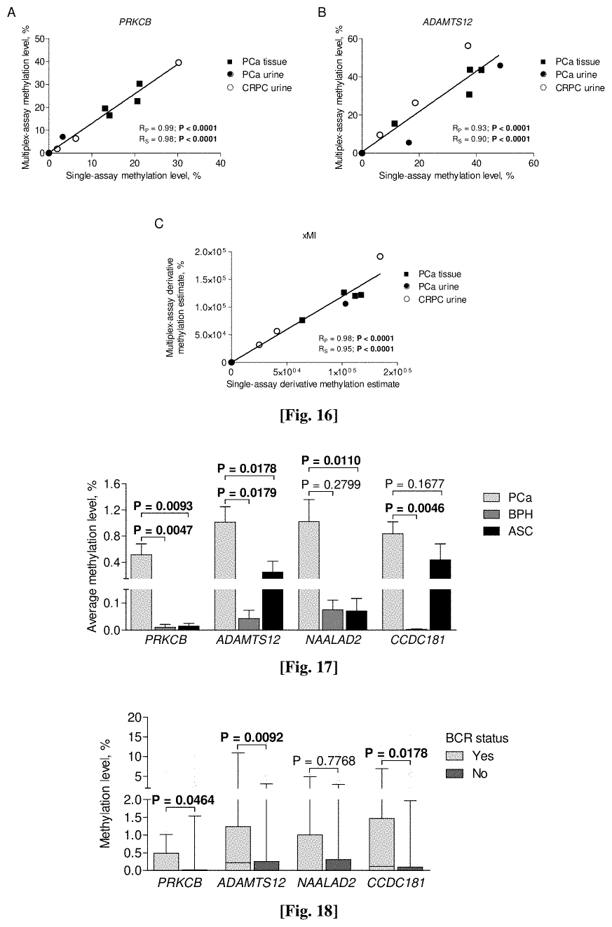

The methylation levels of PRKCB and ADAMTS12, as well as the derivative estimate xMI of their combination, were compared between single-assay and multiplex-assay experiments using the same primers and probes (Table 5). The analysis was performed in randomly selected prostate tumours and urine samples from patients with localized or locally advanced PCa and CRPC (4 samples per group, 12 in total; FIG. 16). Strong correlations (R_{S}≥0.90; P<0.0001) were observed for both biomarkers and their xMI indicating the potential to develop a cost-effective multiple-assay test.

Biomarker Performance in Urine for Non-Invasive Diagnostics of Prostate Cancer

DNA methylation of the genes PRKCB, ADAMTS12, NAALAD2 and CCDC181 was evaluated (single-assay experiments) in voided urine samples collected from the PCa patients diagnosed with localized or locally advanced disease (N=152). Average methylation levels were higher in urine of PCa cases and significantly differed from BPH and/or ASC cases, differentiating patients from controls (FIG. 17).

The methylation frequencies determined in urine samples of PCa, BPH and ASC cases, using each of the thresholds, are provided in Table 14. Methylation frequencies of all the genes were the highest in urine from PCa cases and differed significantly from BPH cases with either threshold (all P<0.0500). For PRKCB and ADAMTS12, the same observation was made by comparing PCa and ASC cases, while NAALAD2 and CCDC181 yielded insignificant results in this particular comparison (Table 14). Nevertheless, the analysis indicated the potential utility of these biomarkers for the non-invasive diagnostics with the post-test probability for PCa detection reaching >99.9% (Table 15). The 0.1 C threshold was used in all further qualitative analysis of the gene methylation levels.

TABLE 14
[Comparison of the gene methylation frequencies in urine
samples obtained from the patients and controls.]

| | Methylation | | | | |
|---|---|---|---|---|---|
| | frequency, % | | | P-value | |
| Gene | PCa | BPH | ASC | PCa vs. BPH | PCa vs. ASC |

ASC-based threshold

| | | | | | |
|---|---|---|---|---|---|
| PRKCB | 30.3 | 3.4 | 6.7 | 0.0020 | 0.0060 |
| ADAMTS12 | 28.9 | 3.4 | 10.0 | 0.0020 | 0.0384 |
| NAALAD2 | 34.2 | 13.8 | 20.0 | 0.0298 | 0.1400 |
| CCDC181 | 24.3 | 0 | 13.3 | 0.0009 | 0.2360 |

BPH-based threshold

| | | | | | |
|---|---|---|---|---|---|
| PRKCB | 32.2 | 6.9 | 6.7 | 0.0058 | 0.0034 |
| ADAMTS12 | 34.2 | 13.8 | 13.3 | 0.0298 | 0.0293 |
| NAALAD2 | 34.2 | 13.8 | 16.7 | 0.0298 | 0.0834 |
| CCDC181 | 39.5 | 10.3 | 23.3 | 0.0024 | 0.1024 |

0.1C threshold

| | | | | | |
|---|---|---|---|---|---|
| PRKCB | 25.7 | 3.4 | 6.7 | 0.0063 | 0.0291 |
| ADAMTS12 | 31.6 | 10.3 | 13.3 | 0.0231 | 0.0477 |
| NAALAD2 | 32.9 | 13.8 | 16.7 | 0.0462 | 0.0855 |
| CCDC181 | 32.2 | 0 | 23.3 | 0.0001 | 0.3924 |

PCa—prostate cancer,
BPH—benign prostatic hyperplasia,
ASC—asymptomatic controls.
Significant P-values are in bold.

TABLE 15
[The post-test probability estimates for diagnosing PCa in an individual
when analysing the particular biomarkers in urine of men suspected of
having PCa.]

| | When pre-test | | When pre-test | |
|---|---|---|---|---|
| | probability is 45.0%* | | probability is 51.8%** | |
| | Post-test | Absolute | Post-test | Absolute |
| Biomarker assay | probability | difference | probability | difference |

ASC-based threshold

| | | | | |
|---|---|---|---|---|
| PRKCB | 87.8% | 42.8% | 90.4% | 38.6% |
| ADAMTS12 | 87.3% | 42.3% | 90.0% | 38.2% |
| NAALAD2 | 67.0% | 22.0% | 72.7% | 20.9% |
| CCDC181 | 99.9% | 54.9% | >99.9% | 48.1% |

BPH-based threshold

| | | | | |
|---|---|---|---|---|
| PRKCB | 79.2% | 34.2% | 83.4% | 31.6% |
| ADAMTS12 | 67.0% | 22.0% | 72.7% | 20.9% |
| NAALAD2 | 67.0% | 22.0% | 72.7% | 20.9% |
| CCDC181 | 75.7% | 30.7% | 80.4% | 28.6% |

0.1C threshold

| | | | | |
|---|---|---|---|---|
| PRKCB | 85.9% | 40.9% | 88.9% | 37.1% |
| ADAMTS12 | 71.4% | 26.4% | 76.6% | 24.8% |
| NAALAD2 | 66.1% | 21.1% | 71.9% | 20.1% |
| CCDC181 | >99.9% | 54.9% | >99.9% | 48.1% |

*According to the 2017 data by the National Health Insurance Fund under the Ministry of Health of Lithuania.
**According to study by Riedinger et al. for the Michigan Urological Surgery Improvement Collaborative (2014) [30]

Prognostic Value of the Biomarkers in Urine of Localized or Locally Advanced Prostate Cancer Patients

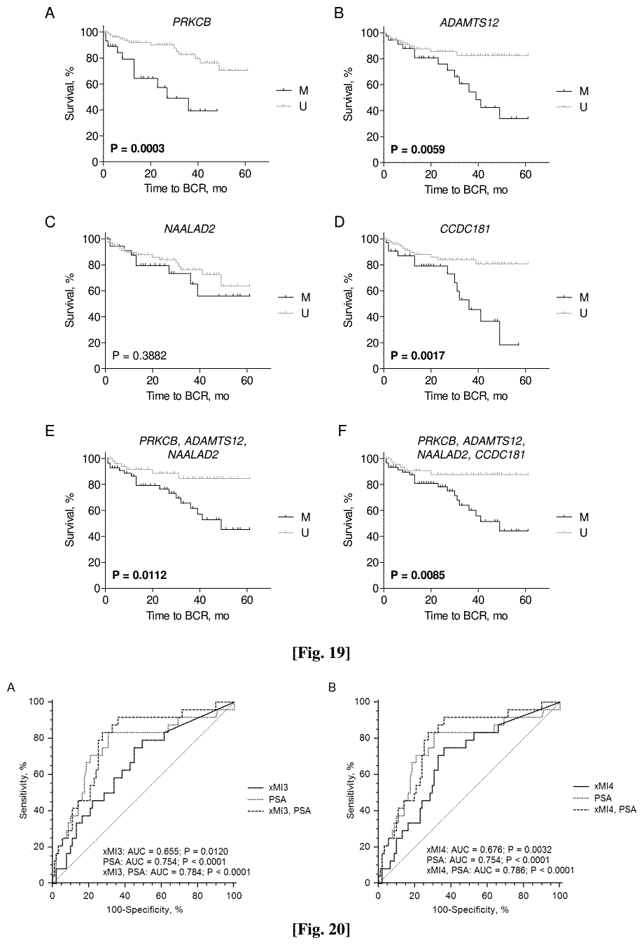

The biomarker methylation in urine was analysed for the potential to predict PCa progression defined as BCR. Higher methylation levels of PRKCB, ADAMTS12 and CCDC181 were observed in the samples of BCR-positive PCa patients as compared to the BCR-negative cases (FIG. 18). Kaplan-Meier curve analysis confirmed the prognostic value of PRKCB, ADAMTS12 and CCDC181 methylation status for BCR-free survival (all P<0.0500; FIG. 19A-D). Furthermore, the combined analysis of PRKCB, ADAMTS12 and NAALAD2 revealed significant association of the methylated gene status with BCR, whereas the inclusion of the gene CCDC181 into the panel provided even better results (P=0.0112 and P=0.0085, respectively; FIGS. 19E and F).

In Cox proportional hazard analysis, the combination of either xMI3 or xMI4 with PSA resulted in significantly increased prognostic power of predicting time to BCR with HR values of up to 53.7, even though none of the methylation biomarkers showed the potential individually and only PSA was predictive of BCR (Table 16). ROC curve analysis revealed moderate-to-high sensitivity and specificity values of xMI3 for predicting BCR, whereas xMI4 estimate, i.e. the inclusion of CCDC181 in the test, provided improved characteristics. The best test performance was observed when the biomarker analysis was combined with PSA (FIG. 20 and Table 17). Moreover, improved test characteristics were observed when methylation was analysed in low ISUP grade or more locally advanced (pT3) PCa cases only (Table 17).

TABLE 16
[Cox proportional hazard analysis of the gene methylation biomarkers
and prostate-specific antigen (PSA) in urine of patients diagnosed with
localized or locally advanced prostate cancer (PCa) for predicting
biochemical disease recurrence (BCR).]

| Biomarker assay(-s) | HR [95% CI] | P-value |
|---|---|---|
| PRKCB | 1.1 [0.9; 1.4] | 0.3817 |
| ADAMTS12 | 1.1 [1.0; 1.2] | 0.1940 |
| NAALAD2 | 1.0 [1.0; 1.1] | 0.6863 |
| CCDC181 | 1.1 [1.0; 1.3] | 0.0757 |
| xMI3 | 1.0 [1.0; 1.1] | 0.1788 |
| xMI4 | 1.0 [1.0; 1.1] | 0.1833 |
| xMI3, PSA | 53.7 [10.3; 280.1] | 0.0001 |
| xMI4, PSA | 53.4 [10.3; 276.2] | 0.0001 |
| PSA | 1.0 [1.0; 1.1] | 0.0006 |

All variables are continuous.
xMI3—the derivative methylation estimate based on biomarkers PRKCB, ADAMTS12 and NAALAD2;
xMI4—the derivate methylation estimate based on the biomarkers PRKCB, ADAMTS12, NAALAD2 and CCDC181;
PSA—prostate-specific antigen;
HR—hazard ratio;
CI—confidence intervals.
Significant P-values are in bold.

TABLE 17
[The test performance characteristics for predicting biochemical
disease recurrence (BCR) when methylation is analysed in urine
of patients diagnosed with localized or locally advanced prostate
cancer (PCa).]

| | | | |
|---|---|---|---|
| Biomarker | | | Youden |
| combination | Sensitivity | Specificity | index |

All PCa cases

| | | | |
|---|---|---|---|
| xMI3 | 75.0% | 55.0% | 0.300 |
| xMI4 | 75.0% | 63.7% | 0.387 |
| xMI3, PSA | 83.3% | 72.5% | 0.558 |
| xMI4, PSA | 83.3% | 72.5% | 0.558 |
| PSA | 83.3% | 69.2% | 0.525 |

ISUP grade group 1 or 2 cases only

| | | | |
|---|---|---|---|
| xMI3 | 78.6% | 55.3% | 0.339 |
| xMI4 | 71.4% | 71.1% | 0.425 |
| xMI3, PSA | 85.7% | 65.8% | 0.515 |
| xMI4, PSA | 85.7% | 65.8% | 0.515 |
| PSA | 78.6% | 71.1% | 0.497 |

pT3 cases only

| | | | |
|---|---|---|---|
| xMI3 | 75.0% | 64.0% | 0.390 |
| xMI4 | 75.0% | 64.0% | 0.390 |
| xMI3, PSA | 100.0% | 72.0% | 0.720 |
| xMI4, PSA | 100.0% | 72.0% | 0.720 |
| PSA | 75.0% | 84.0% | 0.590 |

All variables are continuous.
xMI3—the derivative methylation estimate based on biomarkers PRKCB, ADAMTS12 and NAALAD2;
xMI4—the derivate methylation estimate based on the biomarkers PRKCB, ADAMTS12, NAALAD2 and CCDC181;
PSA—prostate-specific antigen;
ISUP—International Society of Urologic Pathology,
pT—pathological tumour stage.

DNA Methylation Analysis in Urine of Patients Diagnosed with Castration-Resistant Prostate Cancer

Aiming to evaluate the predictive value of the biomarkers, the genes PRKCB, ADAMTS12, NAALAD2 and CCDC181 were analysed in voided urine samples (N=230), collected from patients diagnosed with castration-resistant prostate cancer (CRPC), before initiating the treatment with AA (N=103) and during the treatment (N=127). Additionally, other genes known to be associated with prostate carcinogenesis, namely MT1E, APC and RASSF1 [18,31,32], were included in the analysis.

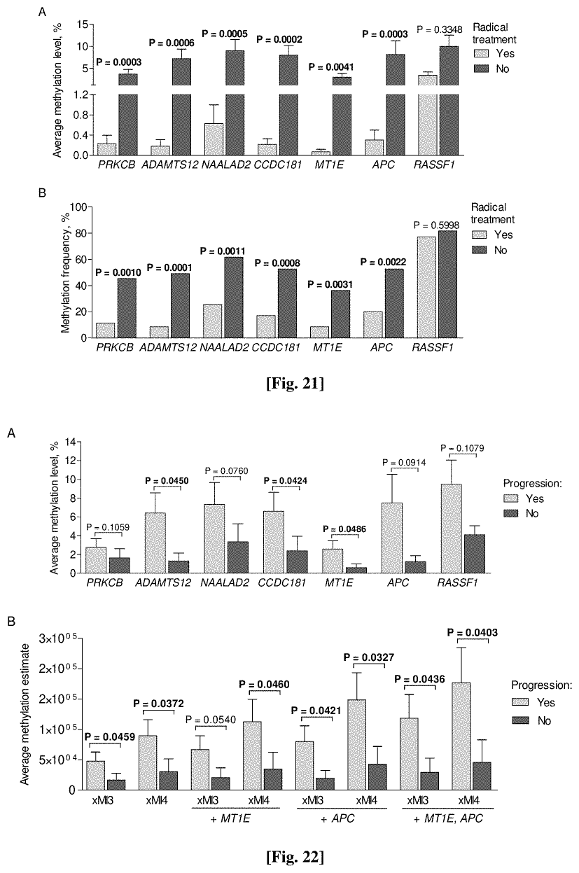

DNA methylation levels of all the genes, except RASSF1, were lower in urine, collected before initiating the 1^{st}-line AA treatment, of CRPC patients who had previously undergone radical treatment (RP or/and radiation therapy) as compared to those who hadn't received such therapy (FIG. 21A). According to the qualitative analysis, methylation was also more frequent in cases who received prior radical treatment (FIG. 21B). Altogether, this proves that metastatic lesions contribute significantly to the amount of methylated DNA detectable in urine, therefore, such epigenetic alterations can be used as indicators of particular PCa characteristics.

Prognosis of Castration-Resistant Prostate Cancer Progression when Undergoing the Treatment with Abiraterone Acetate

Analysing urine samples collected before initiating the AA treatment, higher methylation levels and xMI values were observed in the CRPC cases who experienced disease progression. However, among the individual genes only ADAMTS12, CCDC181 and MT1E showed significant differences, whereas both xMI3 and xMI4 estimates, as well as their various combinations with other genes (MT1E and/or APC), differed significantly between the two groups (FIG. 22). According to the Cox proportional hazard regression analysis, methylation levels of PRKCB, ADAMTS12 and CCDC181 in urine samples collected before initiating the AA treatment were close-to-significant, as well as the xMI3 or xMI4 values, for predicting progression-free survival time of the CRPC patients. The inclusion of the genes MT1E, APC and RASSF1 in the derivative estimates xMI resulted in the significant combined predictor of the disease progression, with the best performance of the combinations of xMI3 or xMI4 with MT1E and APC (Table 18).

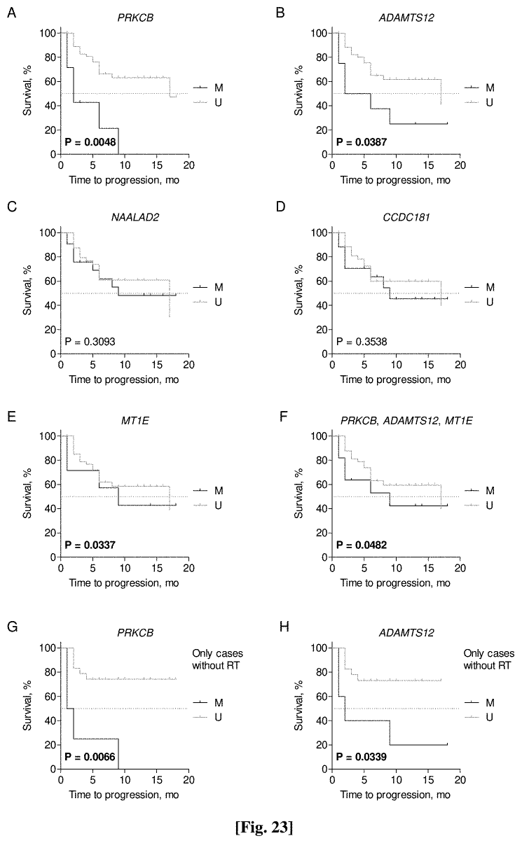

In the Cox models, methylation status of the biomarkers individually did not show significance for predicting progression when analysed in urine collected before initiating the treatment; however, methylated status of PRKCB, as well as MT1E, was predictive of the disease progression when analysed in urine collected after at least 2 mos. of the treatment (Table 18). Methylation status of ADAMTS12 or the combination of PRKCB, ADAMTS12 and MT1E was also close to significant for predicting the progression. Kaplan-Meier curve comparison confirmed the prognostic value of the PRKCB, ADAMTS12 and MT1E biomarker methylation separately or in combination when evaluated in urine collected during the treatment (FIG. 23A-F). Furthermore, in the subgroup of CRPC patients who did not receive prior radical treatment, the methylation status and the methylation level of PRKCB in urine collected during the AA treatment was a significant predictor of the progression-free survival. Also, methylation of ADAMTS12 was associated with the time-to-progression in this subgroup; however, no significance was observed in Cox models for this gene (FIGS. 23G and H, and Table 17). A selection of xMI alternatives showed a tendency of association with the disease progression when analysed in urine collected during the treatment, however, insignificant (Table 18).

TABLE 18
[Cox proportional hazard analysis of the selected gene methylation
biomarkers in urine of patients diagnosed with castration-resistant
prostate cancer (CRPC) for predicting progression-free survival
before and during the treatment withabiraterone acetate (AA).]

| | Before AA treatment | | During AA treatment | |
|---|---|---|---|---|
| | (N = 86) | | (N = 76) | |
| Biomarker assay(-s) | HR [95% CI] | P-value | HR [95% CI] | P-value |

Qualitative variables, all cases

| | | | | |
|---|---|---|---|---|
| PRKCB | 1.6 [0.8; 2.9] | 0.1791 | 3.1 [1.5; 6.7] | 0.0078 |
| ADAMTS12 | 1.2 [0.6;2.3] | 0.5859 | 2.2 [1.0;4.6] | 0.0617 |
| NAALAD2 | 0.8 [0.4; 1.5] | 0.4975 | 1.4 [0.7; 2.6] | 0.3361 |
| CCDC181 | 0.9 [0.5; 1.8] | 0.8699 | 1.3 [0.7;2.6] | 0.3894 |
| MT1E | 1.7 [0.9; 3.3] | 0.1492 | 2.2 [1.1; 4.5] | 0.0485 |
| APC | 0.9 [0.5; 1.7] | 0.8421 | 1.1 [0.5; 2.1] | 0.8455 |
| RASSF1 | 0.6 [0.3; 1.2] | 0.1761 | 1.2 [0.5; 3.1] | 0.7087 |
| PRKCB, | 0.8 [0.6; 2.0] | 0.8100 | 2.0 [1.0; 3.9] | 0.0654 |
| ADAMTS12, MT1E | | | | |

Quantitative variables, all cases

| | | | | |
|---|---|---|---|---|
| PRKCB | 1.0 [1.0; 1.1] | 0.0776 | 1.0 [1.0; 1.1] | 0.0936 |
| ADAMTS12 | 1.0 [1.0; 1.0] | 0.0567 | 1.0 [1.0; 1.0] | 0.5371 |
| NAALAD2 | 1.0 [1.0; 1.0] | 0.1185 | 1.0 [1.0; 1.0] | 0.4138 |
| CCDC181 | 1.0 [1.0; 1.0] | 0.0864 | 1.0 [1.0; 1.0] | 0.3255 |
| MT1E | 1.1 [1.0; 1.1] | 0.0328 | 1.1 [1.0; 1.1] | 0.1433 |
| APC | 1.0 [1.0; 1.0] | 0.0050 | 1.0 [1.0; 1.1] | 0.5743 |
| RASSF1 | 1.0 [1.0; 1.0] | 0.0410 | 1.0 [1.0; 1.0] | 0.2262 |
| xMI3 | 1.0 [1.0; 1.0] | 0.0545 | 1.0 [1.0; 1.0] | 0.0867 |
| xMI4 | 1.0 [1.0; 1.0] | 0.0541 | 1.0 [1.0; 1.0] | 0.0778 |
| xMI3 + MT1E | 1.0 [1.0; 1.0] | 0.0260 | 1.0 [1.0; 1.0] | 0.0751 |
| xMI4 + MT1E | 1.0 [1.0; 1.0] | 0.0260 | 1.0 [1.0; 1.0] | 0.0719 |
| xMI3 + APC | 1.0 [1.0; 1.0] | 0.0115 | 1.0 [1.0; 1.0] | 0.1189 |
| xMI4 + APC | 1.0 [1.0; 1.0] | 0.0141 | 1.0 [1.0; 1.0] | 0.0766 |
| xMI3 + RASSF1 | 1.0 [1.0; 1.0] | 0.0362 | 1.0 [1.0; 1.0] | 0.0701 |
| xMI4 + RASSF1 | 1.0 [1.0; 1.0] | 0.0418 | 1.0 [1.0; 1.0] | 0.0684 |
| xMI3 + MT1E, APC | 1.0 [1.0; 1.0] | 0.0045 | 1.0 [1.0; 1.0] | 0.0742 |
| xMI4 + MT1E, APC | 1.0 [1.0; 1.0] | 0.0066 | 1.0 [1.0; 1.0] | 0.0716 |

Qualitative variables, only cases without RT

| | | | | |
|---|---|---|---|---|
| PRKCB | 1.4 [0.7; 3.2] | 0.3686 | 4.1 [1.5; 11.4] | 0.0127 |
| ADAMTS12 | 1.1 [0.5; 2.5] | 0.7552 | 2.9 [1.1; 7.9] | 0.0531 |

Quantitative variables, only cases without RT

| | | | | |
|---|---|---|---|---|
| PRKCB | 1.0 [1.0; 1.1] | 0.1186 | 1.1 [1.0; 1.1] | 0.0161 |
| ADAMTS12 | 1.0 [1.0; 1.0] | 0.0952 | 1.0 [1.0; 1.0] | 0.4503 |

xMI3—the derivative methylation estimate based on biomarkers PRKCB, ADAMTS12 and NAALAD2;
xMI4—the derivate methylation estimate based on the biomarkers PRKCB, ADAMTS12, NAALAD2 and CCDC181;
HR—hazard ratio;
CI—confidence intervals.
Significant P-values are in bold.

Prediction of Primary and Acquired Resistance to Abiraterone Acetate Treatment

The predictive potential of the biomarkers was analysed according to two different definitions of the treatment resistance commonly used by clinicians. The conventional definition describes the primary resistance as the treatment failure within the first 3 mos. after treatment initiation, while the treatment failure that occurs later is referred to as the acquired resistance. Alternatively, the primary resistance can be defined as the absence of PSA level reduction by ≥50%, which is herein referred to as the PSA progression, whereas the cases with the presence of the PSA reduction by ≥50% are considered as responsive to treatment.

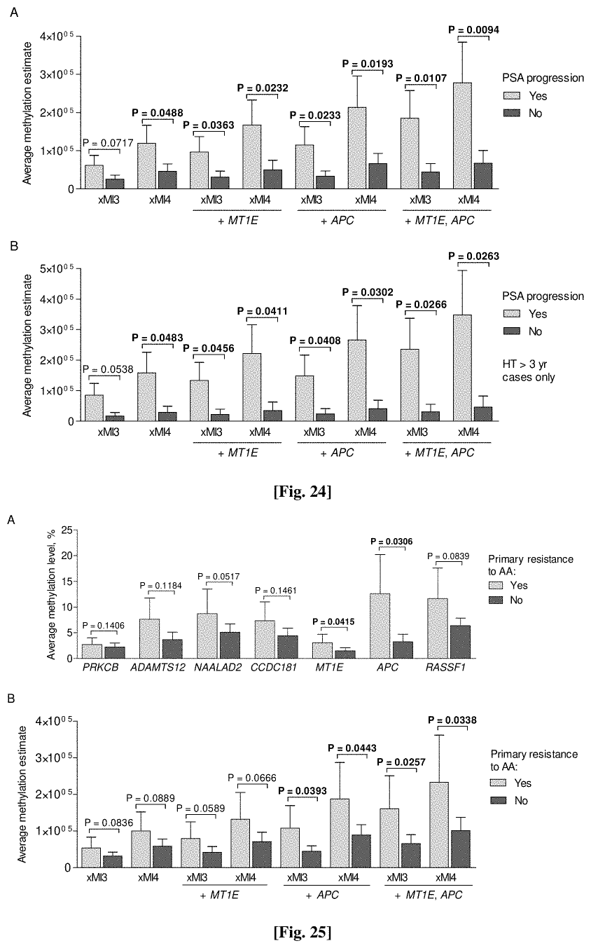

The methylation estimate xMI3 and xMI4 values obtained from urine samples collected before initiating the AA treatment were higher in CRPC cases with the PSA progression. Inclusion of the genes MT1E and/or APC in the xMI estimate resulted in even more significant differences between the groups (FIG. 24A). More specifically, the differences of xMI values between the cases with and without the PSA progression were observed in the subgroup of CRPC patients, who had long response (>3 yrs.) to prior ADT (FIG. 24B). Several individual gene assays also showed significant or close to significant differences but did not outperform the xMI estimates in this group comparison. The qualitative evaluation of the biomarker methylation did not show significant associations (not shown).

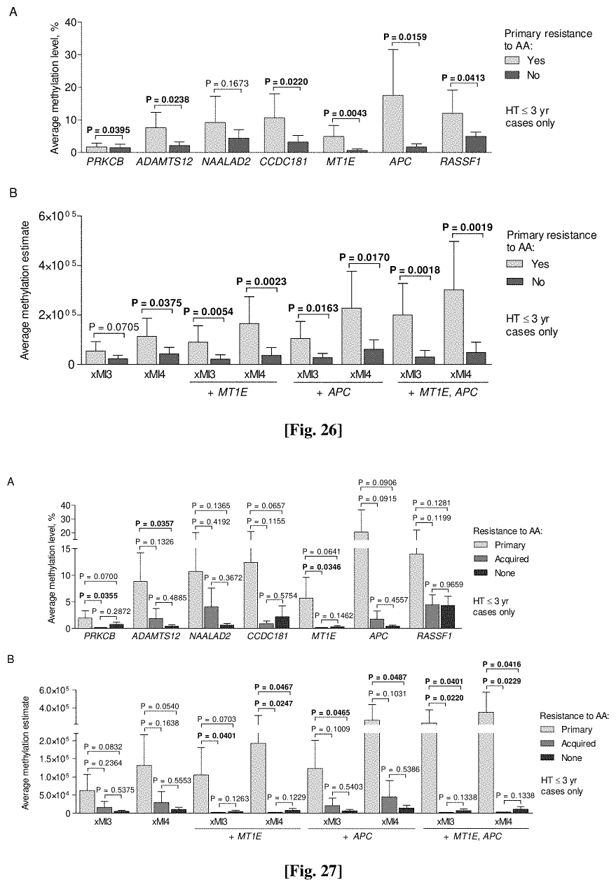

In urine collected before the AA treatment, methylation levels were higher in the CRPC cases that had the primary resistance to AA as compared to other cases; however, only MT1E, APC and RASSF1 showed significant associations, while NAALAD2 was borderline significant (FIG. 25A). The various alternative xMI values indicated that the inclusion of more genes in the panel increased its discriminative power for the identification of CRPC cases with the primary resistance (FIG. 25B). Furthermore, in the subgroup of CRPC patients with short response (≤3 yrs.) to prior HT, the genes PRKCB, ADAMTS12 and CCDC181, together with the previously mentioned MT1E, APC and RASSF1, were methylated at significantly higher levels in the cases with the primary resistance to AA and improved performance of the xMI estimates was observed as compared to the total group (FIG. 26). Moreover, in the subgroup of ADT≤3 yrs. cases, the genes PRKCB and MT1E individually, as well as xMI3 and xMI4 estimates in combination with MT1E and APC or MT1E alone, could distinguish between the cases with the primary and acquired resistance to AA (FIG. 27).

Prognosis of Overall Survival of Castration-Resistant Prostate Cancer Cases

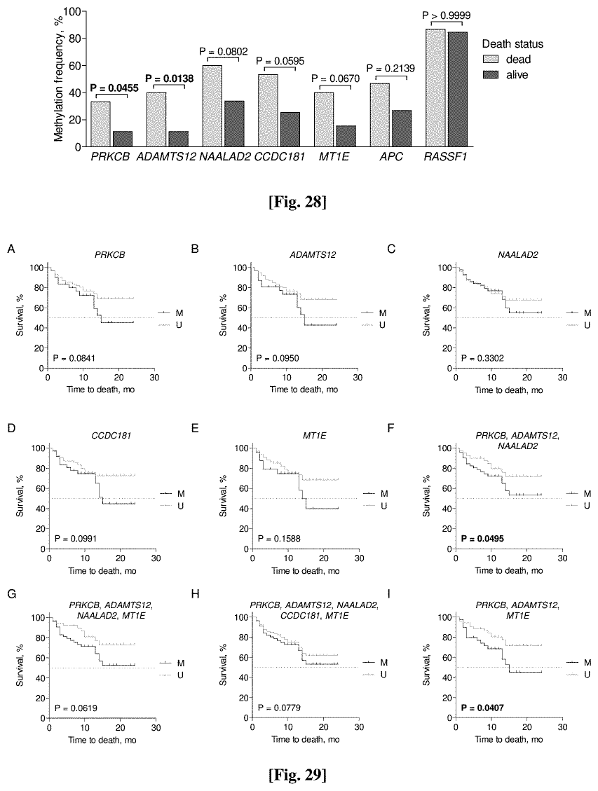

In the overall survival analysis, methylation status of the analysed biomarkers did not show significant differences in urine collected before initiating the treatment with AA (not shown), but higher methylation levels of the genes PRKCB and ADAMTS12 were associated with patient's death when analysed in urine collected at least after 2 mos. of the treatment (FIG. 28). Further analysis revealed the improved performance of the biomarkers when analysed in urine collected at 6±2 mos. of the AA treatment.

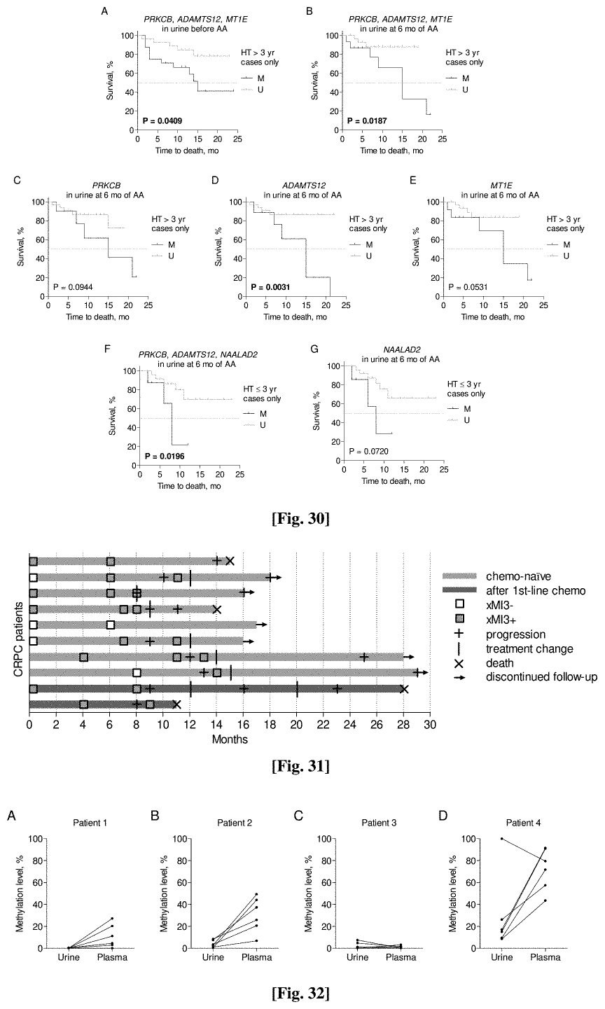

In urine collected before the AA treatment, Kaplan-Meier curve comparisons indicated the potential prognostic value of the biomarker combinations, particularly PRKCB, ADAMTS12 and NAALAD2 or PRKCB, ADAMTS12 and MT1E, although the individual biomarkers lacked significance for the stratification of the cases according to the overall survival (FIG. 29). Furthermore, methylation status according to the latter biomarker panel was also associated with the overall survival of CRPC cases who had long (>3 yrs.) response to ADT (FIG. 30A). According to the Cox proportional hazard analysis, methylation level of NAALAD2 and the xMI3 estimate alone or combined with MT1E were significant predictors of time to death when analysed in the subgroup of CRPC patients who were responsive to ADT for ≤3 yrs. (Table 19).

In urine collected at 6±2 mos. of the treatment, methylation levels of MT1E and APC were significant predictors of the overall survival in Cox proportional hazard analysis. Different biomarkers or their combinations were associated with the overall survival when analysed in the subgroups according to the duration of the response to ADT. Specifically, methylation status of the biomarker combination of PRKCB, ADAMTS12 and MT1E were associated with the overall survival in the cases with ADT>3 yrs.; however, only ADAMTS12 had independent prognostic value, while PRKCB and MT1E showed weak tendencies (FIG. 30B-E). In urine of the patients with ADT≤3 yrs., methylation status of another biomarker panel, i.e. PRKCB, ADAMTS12 and NAALAD2, was associated with the overall survival, although none of the genes individually were predictive of the patient's death, except of a close to significant tendency observed for NAALAD2 (FIGS. 30F and G).

Representative CRPC cases showing the biomarker test result according to the xMI3 value are depicted in FIG. 31.

DNA Methylation Biomarkers in Plasma Samples of CRPC Patients

DNA methylation levels of the biomarkers (PRKCB, ADAMTS12, NAALAD2, CCDC181, MT1E and APC) were analysed in four plasma samples, as an alternative liquid-biopsy option. The analysis indicated that the analysed biomarkers can be successfully detected in patients' plasma (FIG. 32). The comparison of paired urine and plasma samples showed relatively higher methylation levels in the latter indicating the potential need of adjustments in the cut-off value used for qualitative evaluation and/or in the xMI algorithm (Formula 3).

TABLE 19
[Cox proportional hazard analysis of the gene methylation biomarkers in urine for predicting overall survival (time to death) of
patients diagnosed with castration-resistant prostate cancer (CRPC) and undergoing treatment with abiraterone acetate (AA).]

| | Before AA treatment | | | | After 6 mos. of AA treatment | | | |
|---|---|---|---|---|---|---|---|---|
| | All cases (N = 87) | | ADT ≤ 3 yrs. cases only (N = 36) | | All cases (N = 75) | | ADT > 3 yrs. cases only (N = 42) | |
| Biomarker assay (-s) | HR [95% CI] | P-value | HR [95% CI] | P-value | HR [95% CI] | P-value | HR [95% CI] | P-value |
| PRKCB | 1.0 [1.0; 1.1] | 0.8126 | 1.3 [1.0; 1.7] | 0.1398 | 1.0 [1.0; 1.1] | 0.2082 | 1.0 [1.0; 1.1] | 0.1959 |
| ADAMTS12 | 1.0 [1.0; 1.0] | 0.4021 | 1.1 [1.0; 1.1] | 0.0634 | 1.0 [1.0; 1.0] | 0.2626 | 1.0 [1.0; 1.0] | 0.2922 |
| NAALAD2 | 1.0 [1.0; 1.0] | 0.3284 | 1.0 [1.0; 1.1] | 0.0333 | 1.0 [1.0; 1.1] | 0.1870 | 1.0 [1.0; 1.1] | 0.1656 |
| CCDC181 | 1.0 [1.0; 1.1] | 0.3187 | 1.0 [1.0; 1.1] | 0.2179 | 1.0 [1.0; 1.1] | 0.1641 | 1.0 [1.0; 1.0] | 0.1651 |
| MT1E | 1.0 [1.0; 1.1] | 0.4849 | 1.1 [1.0; 1.2] | 0.0886 | 1.1 [1.0; 1.1] | 0.0200 | 1.1 [1.0; 1.2] | 0.0218 |
| APC | 1.0 [1.0; 1.0] | 0.5449 | 1.0 [1.0; 1.0] | 0.6206 | 1.0 [1.0; 1.1] | 0.0208 | 1.0 [1.0; 1.1] | 0.0229 |
| RASSF1 | 1.0 [1.0; 1.1] | 0.8079 | 1.0 [1.0; 1.1] | 0.3539 | 1.0 [1.0; 1.1] | 0.1227 | 1.0 [1.0; 1.1] | 0.0229 |
| xMI3 | 1.0 [1.0; 1.0] | 0.4141 | 1.0 [1.0; 1.0] | 0.0333 | 1.0 [1.0; 1.0] | 0.2041 | 1.0 [1.0; 1.0] | 0.2053 |
| xMI4 | 1.0 [1.0; 1.0] | 0.3733 | 1.0 [1.0; 1.0] | 0.0528 | 1.0 [1.0; 1.0] | 0.1829 | 1.0 [1.0; 1.0] | 0.1836 |
| xMI3 + MT1E | 1.0 [1.0; 1.0] | 0.6275 | 1.0 [1.0; 1.0] | 0.0892 | 1.0 [1.0; 1.0] | 0.1517 | 1.0 [1.0; 1.0] | 0.1504 |
| xMI4 + MT1E | 1.0 [1.0; 1.0] | 0.5928 | 1.0 [1.0; 1.0] | 0.1380 | 1.0 [1.0; 1.0] | 0.1512 | 1.0 [1.0; 1.0] | 0.1504 |
| xMI3 + APC | 1.0 [1.0; 1.0] | 0.7110 | 1.0 [1.0; 1.0] | 0.1051 | 1.0 [1.0; 1.0] | 0.0991 | 1.0 [1.0; 1.0] | 0.0958 |
| xMI4 + APC | 1.0 [1.0; 1.0] | 0.6034 | 1.0 [1.0; 1.0] | 0.1308 | 1.0 [1.0; 1.0] | 0.1258 | 1.0 [1.0; 1.0] | 0.1234 |
| xMI3 + RASSF1 | 1.0 [1.0; 1.0] | 0.4903 | 1.0 [1.0; 1.0] | 0.0476 | 1.0 [1.0; 1.0] | 0.1713 | 1.0 [1.0; 1.0] | 0.1695 |
| xMI4 + RASSF1 | 1.0 [1.0; 1.0] | 0.4462 | 1.0 [1.0; 1.0] | 0.0668 | 1.0 [1.0; 1.0] | 0.1653 | 1.0 [1.0; 1.0] | 0.1642 |
| xMI3 + MT1E, APC | 1.0 [1.0; 1.0] | 0.9216 | 1.0 [1.0; 1.0] | 0.2491 | 1.0 [1.0; 1.0] | 0.0955 | 1.0 [1.0; 1.0] | 0.0923 |
| xMI4 + MT1E, APC | 1.0 [1.0; 1.0] | 0.8343 | 1.0 [1.0; 1.0] | 0.3055 | 1.0 [1.0; 1.0] | 0.1085 | 1.0 [1.0; 1.0] | 0.1059 |

All variables are continuous.
xMI3—the derivative methylation estimate based on biomarkers PRKCB, ADAMTS12 and NAALAD2;
xMI4—the derivate methylation estimate based on the biomarkers PRKCB, ADAMTS12, NAALAD2 and CCDC181;
AA—abiraterone acetate;
ADT—androgen deprivation therapy;
HR—hazard ratio;
CI—confidence intervals.
Significant P-values are in bold.

### REFERENCES

1. Lin, D W, Porter, M, and Montgomery, B. Treatment and survival outcomes in young men diagnosed with prostate cancer: a population based cohort study. Cancer, 115, 13 (2009), 2863-2871.
2. Markozannes, G, Tzoulaki, I, Karli, D et al. Diet, Body Size, Physical Activity and Risk of Prostate. European Journal of Cancer, 69 (2016), 61-69.
3. Serenaite, I, Daniunaite, K, Jankevicius, F, Laurinavicius, A, Petroska, D, Lazutka, J R, and Jarmalaite, S. Heterogeneity of DNA methylation in multifocal prostate cancer. Virchows Arch, 466, 1 (2015), 53-59.
4. Cooper, C S, Eeles, R, Wedge, D C et al. Analysis of the genetic phylogeny of multifocal prostate cancer identifies multiple independent clonal expansions in neoplastic and morphologically normal prostate tissue. Nat Genet, 47, 4 (2015), 367-372.
5. Lindberg, J, Klevebring, D, Liu, W et al. Exome sequencing of prostate cancer supports the hypothesis of independent tumour. Eur Urol, 63, 2 (2013), 347-353.
6. Shen, M M and Abate-Shen, C. Molecular genetics of prostate cancer: new prospects for old challenges (2010), 1967-2000.
7. Sartor, A O, Hricak, H, Wheeler, T M et al. Evaluating localized prostate cancer and identifying candidates for focal therapy. Urology, 72, 6 Suppl (2008), S12-24.
8. Mottet, N, Bellmunt, J, Bolla, M et al. EAU-ESTRO-SIOG Guidelines on Prostate Cancer. Part 1: Screening, Diagnosis, and Local Treatment with Curative Intent. Eur Urol, 71, 4 (2017), 618-629.
9. Katzenwadel, A and Wolf, P. Androgen deprivation of prostate cancer: Leading to a therapeutic dead end. Cancer Lett, 367, 1 (2015), 12-17.
10. Jones, P A. Functions of DNA methylation: islands, start sites, gene bodies and beyond. Nat Rev Genet, 13, 7 (2012), 484-492.
11. Shen, H and Laird, P W. Interplay between the cancer genome and epigenome. Cell, 153, 1 (2013), 38-55.
12. Stirzaker, C, Taberlay, P C, Statham, A L, and Clark, S J. Mining cancer methylomes: prospects and challenges. Trends Genet, 30, 2 (2014), 75-84.
13. Vener, T, Derecho, C, Baden, J et al. Development of a multiplexed urine assay for prostate cancer diagnosis. Clin Chem, 54, 5 (2008), 874-882.
14. Sunami, E, Shinozaki, M, Higano, C S et al. A Multimarker Circulating DNA Assay for Assessing Prostate Cancer Patients' Blood. Clin Chem, 55, 3 (2009), 559-567.
15. Payne, S R, Serth, J, Schostak, M et al. DNA methylation biomarkers of prostate cancer: confirmation of candidates and evidence urine is the most sensitive body fluid for non-invasive detection. Prostate, 69, 12 (2009), 1257-1269.
16. Di Meo, A, Bartlett, J, Cheng, Y, Pasic, M D, and Yousef, G M. Liquid biopsy: a step forward towards precision medicine in urologic malignancies. Mol Cancer, 16, 1 (2017), 80.
17. Kurdyukov, S and Bullock, M. DNA Methylation Analysis: Choosing the Right Method. Biology (Basel), 5, 1 (2016), 3.
18. Daniunaite, K, Jarmalaite, S, Kalinauskaite, N, Petroska, D, Laurinavicius, A, Lazutka, J R, and Jankevicius, F. Prognostic value of RASSF1 promoter methylation in prostate cancer. J Urol, 192 (2014), 1849-1855.
19. Buttigliero, C, Tucci, M, Bertaglia, V, Vignani, F, Bironzo, P, Di Maio, M, and Scagliotti, G V. Understanding and overcoming the mechanisms of primary and acquired resistance to abiraterone and enzalutamide in castration resistant prostate cancer. Cancer Treat Rev, 41, 10 (2015), 884-892.
20. Subramanian, A, Tamayo, P, Mootha, V K et al. Gene set enrichment analysis: a knowledge-based approach for interpreting genome-wide expression profiles. Proc Natl Acad Sci USA, 102, 43 (2005), 15545-15550.
21. Liberzon, A, Birger, C, Thorvaldsdottir, H, Ghandi, M, Mesirov, J P, and Tamayo, P. The Molecular Signatures Database (MSigDB) hallmark gene set collection. Cell Syst, 1, 6 (2015), 417-425.
22. Daniunaite, K, Dubikaityte, M, Gibas, P et al. Clinical significance of miRNA host gene promoter methylation in prostate cancer. Hum Mol Genet, 52, 5 (2017), 2451-2461.
23. Li, L C and Dahiya, R. MethPrimer: designing primers for methylation PCRS. Bioinformatics, 18, 11(2002), 1427-1431.
24. Lehmann, U, Langer, F, Feist, H, Gockner, S, Hasemeier, B, and Kreipe, H. Quantitative assessment of promoter hypermethylation during breast cancer development. Am J Pathol, 160, 2 (2002), 605-612.
25. Brait, M, Ford, J G, Papaiahgari, S et al. Association between lifestyle factors and CpG island methylation in a cancer-free population. Cancer Epidemiol Blomarkers Prev, 18, 11 (2009), 2984-91.
26. The Cancer Genome Atlas Research Network. The Molecular Taxonomy of Primary Prostate Cancer. Cell, 163, 4 (2015), 1011-1025.
27. Cerami, E, Gao, J, Dogrusoz, U et al. The cBio cancer genomics portal: an open platform for exploring multidimensional cancer genomics data. Cancer Discov, 2, 5 (2012), 401-404.
28. Huang, W Y, Hsu, S D, Huang, H Y, Sun, Y M, Chou, C H, Weng, S L, and Huang, H D. MethHC: a database of DNA methylation and gene expression in human cancer. Nucleic Acids Res, 43, Database issue (2015), D856-D861.
29. Haldrup, C, Mundbjerg, K, Vestergaard, E M et al. DNA methylation signatures for prediction of biochemical recurrence after radical prostatectomy of clinically localized prostate cancer. J Clin Oncol, 31, 26 (2013), 3250-3258.
30. Riedinger, C B, Womble, P R, Linsell, S M, Ye, Z, Montie, J E, Miller, D C, and et al. Variation in prostate cancer detection rates in a statewide quality improvement collaborative. J Urol, 192, 2 (2014), 373-378.
31. Demidenko, R, Daniunaite, K, Bakavicius, A et al. Decreased expression of MT1E is a potential biomarker of prostate cancer progression. Oncotarget, 8, 37 (2017), 61709-61718.
32. Hendriks, R J, Dijkstra, S, Smit, F P, Vandersmissen, J, Van de Voorde, H, and Mulders, P F A, et al. Epigenetic markers in circulating cell-free DNA as prognostic markers for survival of castration-resistant prostate cancer patients. Prostate, 78 (2018), 336-342.

1-23. (canceled)

24. A method for detecting PCa in an individual diagnosed with PCa, suspected of having PCa or having predisposition to PCa, comprising:
providing a sample obtained from said individual;
determining in the sample, a DNA methylation status of at least two biomarkers one of which is NAALAD2 and at least one other biomarker is selected from a group of nucleotide sequences, said group consisting of:
a) a first set of nucleotide sequences consisting of PRKCB (SEQ ID NO: 1 or 8), ADAMTS12 (SEQ ID NO: 2 or 9), NAALAD2 (SEQ ID NO: 3 or 10), FILIP1L (SEQ ID NO: 4) and KCTD8 (SEQ ID NO: 6);
b) nucleotide sequences being complementary/antisense to the first set of nucleotide sequences;
c) a nucleotide sequence having at least 90% sequence identity to any one of a) or b); and
d) a fragment of any one of a), b) or c), wherein the fragment comprises at least 16 consecutive nucleotides of any one of a), b) or c);
identifying the sample as containing cancerous cells, precursor to cancerous, or predisposed to cancer, or as containing nucleic acids from cells that are cancerous, precursor to cancerous, or predisposed to cancer if DNA methylation is detected by the at least two biomarkers, one of which is NAALAD2, in the sample; and
identifying the individual as having PCa if DNA methylation is detected by the at least two biomarkers, one of which is NAALAD2, in the sample.

25. The method of claim 24, wherein the sample comprises a prostate tissue sample or a body fluid sample, wherein the body fluid sample is a urine sample, a blood sample, a plasma sample, a serum sample or a secretion sample.

26. The method of claim 24, which comprises detection of DNA methylation status of NAALAD2 and one, two, three, or four of the biomarkers.

27. The method of claim 24, wherein DNA methylation status is detected utilizing primers and a probe for NAALAD2, wherein at least two of the primers or probes are for PRKCB, ADAMTS12, FILIP1L or KCTD8, selected from the nucleotide sequences set forth in SEQ ID NOs: 16-64.

28. A method for assisting in treatment selection or patient's monitoring in an individual diagnosed with PCa or CRPC, comprising:
providing a sample obtained from said individual; and
determining DNA methylation level of two biomarkers in the sample, wherein one biomarker is NAALAD2, or all three biomarkers selected from a group of nucleotide sequences, wherein the group consists of:
a) nucleotide sequences consisting of PRKCB (SEQ ID NO: 1 or 8), ADAMTS12 (SEQ ID NO: 2 or 9), NAALAD2 (SEQ ID NO: 3 or 10);
b) nucleotide sequences being complementary to said nucleotide sequences;
c) nucleotide sequence having at least 90% sequence identity to any one of a) or b); and
d) a fragment of any one of a), b) or c), wherein the fragment comprises at least 16 consecutive nucleotides of any one of a), b) or c).

29. The method according to claim 28, wherein the sample comprises a prostate tissue sample or a metastatic tissue sample, or a body fluid sample, wherein the body fluid sample is a urine sample, a blood sample, a plasma sample, a serum sample or secretion sample.

30. The method according to claim 28, which comprises determining DNA methylation level and/or DNA methylation status of NAALAD2 and one or two of the other biomarkers, wherein the DNA methylation level and/or DNA methylation status is indicative of the disease progression or poor outcome.

31. The method according to claim 28, which comprises determining DNA methylation level of NAALAD2 and one other biomarkers, wherein the DNA methylation level is indicative of the effectiveness of treatment with abiraterone acetate, response to the treatment, resistance to the treatment or development of a resistance to the treatment.

32. The method according to claim 28, wherein DNA methylation level is detected utilizing at least two of primers or probes selected from the nucleotide sequences set forth in SEQ ID NOs: 44-64.

33. The method according to claim 24, which is used in combination with other molecular analysis-based methods, urinary cytology analysis, or clinical-pathological individual's characteristics.

34. The method of claim 27, wherein a primer or a primer pair are used for determining the DNA methylation status of NAALAD2 and at least one biomarker selected from a group consisting of PRKCB, ADAMTS12, FILIP1L, ZMIZ1 and KCTD8, wherein the primer or the primer pair comprise one or more nucleotide sequence selected from SEQ ID NOs: 16-39 and SEQ ID NOs: 44-52.

35. The method of claim 32, wherein a primer pair or a combination of at least two primers are used for determining the DNA methylation level of NAALAD2 and at least one other biomarker selected from the group of PRKCB and ADAMTS12, wherein the primer pair or the primer combination comprise one or more nucleotide sequences selected from SEQ ID NOs: 44-45 for PRKCB, SEQ ID NOs: 47-48 for ADAMTS12 and SEQ ID NOs: 50-51 for NAALAD2.

36. The method of claim 32, wherein a probe is used for determining the DNA methylation level of NAALAD2 and at least one of the biomarkers from a group consisting of PRKCB and ADAMTS12, wherein the probe comprises the nucleotide sequence selected from SEQ ID NO: 46 for PRKCB, SEQ ID NO: 49 for ADAMTS12 and SEQ ID NO: 52 for NAALAD2 and a fluorescent label, a fluorescent quenching agent or both.

37. A kit configured to implement the method of claim 24 for determining the DNA methylation status in a sample containing prostate tissue, prostate cells, nucleic acids from prostate cells, body fluid or nucleic acids from body fluid, wherein DNA methylation status is determined by NAALAD2 (SEQ ID NO: 3 or 10) and at least one biomarker of a group consisting of PRKCB (SEQ ID NO: 1 or 8), ADAMTS12 (SEQ ID NO: 2 or 9), FILIP1L (SEQ ID NO: 4), ZMIZ1 (SEQ ID NO: 5) and KCTD8 (SEQ ID NO: 6).

38. The kit according to claim 37, which comprises means for detecting DNA methylation status in the group of biomarkers comprising three, four, or five of the biomarkers, one of which is NAALAD2.

39. The kit according to claim 37, wherein the means for determining DNA methylation status comprises methylation specific polymerase chain reaction using amplifications primers, wherein the primers comprise at least one primer pair or any two of the primers selected from SEQ ID NOs: 16-39 and SEQ ID NOs: 44-52.

40. A kit configured to implement the method of claim 28 for determining DNA methylation level in a sample containing prostate tissue, prostate cells, nucleic acids from prostate cells, body fluid or nucleic acids from body fluid, wherein DNA methylation level is determined by NAALAD2 (SEQ ID NO: 10) and at least one of the biomarkers of the group consisting of PRKCB (SEQ ID NO: 8) and ADAMTS12 (SEQ ID NO: 9).

41. The kit of claim 40, which comprises means for determining the DNA methylation level in the group of biomarkers comprising at least two, three, four, five, six or seven genes.

42. The kit of claim 40, wherein the means for determining DNA methylation level comprises real-time or end-point methylation specific polymerase chain reaction using amplifications primers, wherein the primers comprise at least one of the primers selected from SEQ ID NOs: 44-45, SEQ ID NOs: 47-48 and SEQ ID NOs: 50-51.

43. The kit of claim 42, wherein the kit comprises at least two probes selected from SEQ ID NO: 46, SEQ ID NO: 49 and SEQ ID NO: 52.
